SEQUENCE LISTING (22 sequences)

SEQ ID NO: 1 - protein, 389 aa, synthetic construct
MTGQVQLQESGGGLVQAGGSLRLSCAASGFTFDDYAIGWFRQAPGKEREGVSCINNSDGSGYVADSVKGRFTISSDNAKNTVYLQMNSLKPEDTAVYYCAAAGDGGCYYGLDYWGKGTQVTVSSGGLECKLSGSGSGSDDPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQGDHHHHHH

SEQ ID NO: 2 - DNA, 1170 bp, synthetic construct
  source 1..1170: mol_type = other DNA
atgaccggtcaggttcagttacaggagtccggtgggggactggtgcaggccgggggttctctgcgccttagctgcgctgcctccggtttcaccttcgatgattacgctattggctggttccgtcaggctcctggcaaggagcgtgaaggagtatcctgtatcaacaattcggacgggagtgggtatgtagcggacagtgttaaaggtcgctttacgatttcctccgacaacgccaagaacaccgtctaccttcaaatgaactccctgaagccagaagataccgctgtttattactgtgccgcagcgggtgacggtggttgttactacggcttggactactggggtaaaggaacccaggttactgtctcatccgggggactggaatgcaagctttcagggtcaggtagtggatctgatgatccgccgcagagtccatgggatcgcgtgaaggacctggccactgtgtacgtggatgtgctcaaagacagcggcagagactatgtgtctcagtttgaaggatccgccttgggcaaacaattgaaccttaagctgctggacaactgggacagcgtgacgtccaccttcagcaagctgcgcgaacagctcggccctgtgacccaggaattctgggataacctggaaaaggagacagagggcctgcgccaggagatgagcaaggatctggaggaggtgaaggccaaggtgcagccgtacctggacgacttccagaagaagtggcaggaggagatggagctctaccgccagaaggtggagccgctgcgcgcagagctgcaggagggcgcgcgccagaagctgcacgagctgcaagagaagctgagcccactgggcgaggagatgcgcgaccgcgcgcgcgcccatgtcgacgcgctgcgcacgcatctggcgccgtacagcgacgagctgcgccagcgcttagcggcgcgccttgaggctctcaaggagaacggcggggcccgcctggccgagtaccacgccaaggccaccgagcatctgagcacgctcagcgagaaggccaagccggcgctcgaggacctgcgccaaggcctgctgccggtgctggagagcttcaaggtcagcttcctgagcgctctggaagagtacactaagaagcttaacacccagggtgatcatcatcatcatcatcattaa

SEQ ID NO: 3 - protein, 408 aa, synthetic construct
MDDPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQGSGSGSGSGSGTAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGSGSGSGSGSGASHHHHHH

SEQ ID NO: 4 - DNA, 1227 bp, synthetic construct
  source 1..1227: mol_type = other DNA
atggatgatccacctcaaagtccatgggatcgcgtgaaggacctggccactgtgtacgtggatgtgctcaaagacagcggcagagactatgtgtctcagtttgaaggatccgccttgggcaaacaattgaaccttaagctgctggacaactgggacagcgtgacgtccaccttcagcaagctgcgcgaacagctcggccctgtgacccaggaattctgggataacctggaaaaggagacagagggcctgcgccaggagatgagcaaggatctggaggaggtgaaggccaaggtgcagccgtacctggacgacttccagaagaagtggcaggaggaaatggagttatatcgccaaaaggtcgagccgctgcgcgcagagctgcaggagggcgcgcgccagaagctgcacgagctgcaagagaagctgagcccattaggtgaagaaatgcgtgaccgcgcgcgcgcacatgtcgacgcattacgcacacatttagcgccgtactctgacgagctgcgccagcgcttagcggcgcgccttgaggctctcaaggagaacggtggagctcgcttagcagaatatcacgccaaggccaccgagcatctgagcacgctcagcgagaaggccaagccggcgctcgaggacctgcgccaaggcctgctgccggtgctggagagcttcaaggtcagcttcctgagcgctctggaagagtacactaagaagcttaacacccagggttctggtagcggctctggttctggttctggtaccgcgccgaccagcagcagcaccaagaagacccagctgcaactggaacacctgctgctggacctgcaaatgattctgaacggtatcaacaactacaagaacccgaaactgacccgtatgctgaccttcaagttttatatgccgaagaaagcgaccgagctgaagcacctgcaatgcctggaggaagagctgaaaccgctggaagaggtgctgaacctggcgcaaagcaagaacttccacctgcgtccgcgtgacctgatcagcaacattaacgtgatcgttctggaactgaaaggcagcgagaccacctttatgtgcgaatatgcggatgagaccgcgaccattgttgagttcctgaaccgttggatcaccttttgccagagcatcattagcaccctgaccggctctggtagcggctctggttctggttctggtgctagccatcaccatcaccatcattaa

SEQ ID NO: 5 - protein, 408 aa, synthetic construct
MDDPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQGSGSGSGSGSGTAPTSSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGSGSGSGSGSGASHHHHHH

SEQ ID NO: 6 - DNA, 1227 bp, synthetic construct
  source 1..1227: mol_type = other DNA
atggatgatccacctcaaagtccatgggatcgcgtgaaggacctggccactgtgtacgtggatgtgctcaaagacagcggcagagactatgtgtctcagtttgaaggatccgccttgggcaaacaattgaaccttaagctgctggacaactgggacagcgtgacgtccaccttcagcaagctgcgcgaacagctcggccctgtgacccaggaattctgggataacctggaaaaggagacagagggcctgcgccaggagatgagcaaggatctggaggaggtgaaggccaaggtgcagccgtacctggacgacttccagaagaagtggcaggaggaaatggagttatatcgccaaaaggtcgagccgctgcgcgcagagctgcaggagggcgcgcgccagaagctgcacgagctgcaagagaagctgagcccattaggtgaagaaatgcgtgaccgcgcgcgcgcacatgtcgacgcattacgcacacatttagcgccgtactctgacgagctgcgccagcgcttagcggcgcgccttgaggctctcaaggagaacggtggagctcgcttagcagaatatcacgccaaggccaccgagcatctgagcacgctcagcgagaaggccaagccggcgctcgaggacctgcgccaaggcctgctgccggtgctggagagcttcaaggtcagcttcctgagcgctctggaagagtacactaagaagcttaacacccagggttctggtagcggctctggttctggttctggtaccgcgccgaccagcagcagcaccaagaagacccagctgcaactggaacacctgctgctggacctgcaaatgattctgaacggtatcaacaactacaagaacccgaaactgacccgtatgctgaccgcgaagtttgctatgccgaagaaagcgaccgagctgaagcacctgcaatgcctggaggaagagctgaaaccgctggaagaggtgctgaacggggcgcaaagcaagaacttccacctgcgtccgcgtgacctgatcagcaacattaacgtgatcgttctggaactgaaaggcagcgagaccacctttatgtgcgaatatgcggatgagaccgcgaccattgttgagttcctgaaccgttggatcacctttgcccagagcatcattagcaccctgaccggctctggtagcggctctggttctggttctggtgctagccatcaccatcaccatcattaa

SEQ ID NO: 7 - protein, 389 aa, synthetic construct
MTGQVQLQESGGGLVQAGGSLRLSCAASGFTFDDYAIGWFRQAPGKEREGVSCINNSDGSGYVADSVKGRFTISSDNAKNTVYLQMNSLKPEDTAVYYCAAAGDGGCYYGLDYWGKGTQVTVSSGGLEVTRSGSGSGSDDPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQGDHHHHHH

SEQ ID NO: 8 - DNA, 1170 bp, synthetic construct
  source 1..1170: mol_type = other DNA
atgaccggtcaggttcagttacaggagtccggtgggggactggtgcaggccgggggttctctgcgccttagctgcgctgcctccggtttcaccttcgatgattacgctattggctggttccgtcaggctcctggcaaggagcgtgaaggagtatcctgtatcaacaattcggacgggagtgggtatgtagcggacagtgttaaaggtcgctttacgatttcctccgacaacgccaagaacaccgtctaccttcaaatgaactccctgaagccagaagataccgctgtttattactgtgccgcagcgggtgacggtggttgttactacggcttggactactggggtaaaggaacccaggttactgtctcatccgggggactggaagtgacgcgttcagggtcaggtagtggatctgatgatccgccgcagagtccatgggatcgcgtgaaggacctggccactgtgtacgtggatgtgctcaaagacagcggcagagactatgtgtctcagtttgaaggatccgccttgggcaaacaattgaaccttaagctgctggacaactgggacagcgtgacgtccaccttcagcaagctgcgcgaacagctcggccctgtgacccaggaattctgggataacctggaaaaggagacagagggcctgcgccaggagatgagcaaggatctggaggaggtgaaggccaaggtgcagccgtacctggacgacttccagaagaagtggcaggaggagatggagctctaccgccagaaggtggagccgctgcgcgcagagctgcaggagggcgcgcgccagaagctgcacgagctgcaagagaagctgagcccactgggcgaggagatgcgcgaccgcgcgcgcgcccatgtcgacgcgctgcgcacgcatctggcgccgtacagcgacgagctgcgccagcgcttagcggcgcgccttgaggctctcaaggagaacggcggggcccgcctggccgagtaccacgccaaggccaccgagcatctgagcacgctcagcgagaaggccaagccggcgctcgaggacctgcgccaaggcctgctgccggtgctggagagcttcaaggtcagcttcctgagcgctctggaagagtacactaagaagcttaacacccagggtgatcatcatcatcatcatcattaa

SEQ ID NO: 9 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 10 - protein, 251 aa, synthetic construct
GLVPRGCIDDPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 11 - protein, 251 aa, synthetic construct
GLVPRGSIDDPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLCPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 12 - protein, 251 aa, synthetic construct
GLVPRGSIDDPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLCALEEYTKKLNTQ

SEQ ID NO: 13 - protein, 249 aa, synthetic construct
RHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 14 - protein, 251 aa, synthetic construct
GLVPRGSIDDPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 15 - protein, 257 aa, synthetic construct
HHHHHHGLVPRGSIDDPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 16 - protein, 10 aa, synthetic construct
GLVPRGSIDD

SEQ ID NO: 17 - DNA, 804 bp, Homo sapiens
  source 1..804: mol_type = unassigned DNA
atgaaagctgcggtgctgaccttggccgtgctcttcctgacggggagccaggctcggcatttctggcagcaagatgaacccccccagagcccctgggatcgagtgaaggacctggccactgtgtacgtggatgtgctcaaagacagcggcagagactatgtgtcccagtttgaaggctccgccttgggaaaacagctaaacctaaagctccttgacaactgggacagcgtgacctccaccttcagcaagctgcgcgaacagctcggccctgtgacccaggagttctgggataacctggaaaaggagacagagggcctgaggcaggagatgagcaaggatctggaggaggtgaaggccaaggtgcagccctacctggacgacttccagaagaagtggcaggaggagatggagctctaccgccagaaggtggagccgctgcgcgcagagctccaagagggcgcgcgccagaagctgcacgagctgcaagagaagctgagcccactgggcgaggagatgcgcgaccgcgcgcgcgcccatgtggacgcgctgcgcacgcatctggccccctacagcgacgagctgcgccagcgcttggccgcgcgccttgaggctctcaaggagaacggcggcgccagactggccgagtaccacgccaaggccaccgagcatctgagcacgctcagcgagaaggccaagcccgcgctcgaggacctccgccaaggcctgctgcccgtgctggagagcttcaaggtcagcttcctgagcgctctcgaggagtacactaagaagctcaacacccagtga

SEQ ID NO: 18 - DNA, 756 bp, synthetic construct
  source 1..756: mol_type = other DNA
ggcctggtgccgcgcggcagcatcgatgatccgccgcagagtccatgggatcgcgtgaaggacctggccactgtgtacgtggatgtgctcaaagacagcggcagagactatgtgtctcagtttgaaggatccgccttgggcaaacaattgaaccttaagctgctggacaactgggacagcgtgacgtccaccttcagcaagctgcgcgaacagctcggccctgtgacccaggaattctgggataacctggaaaaggagacagagggcctgcgccaggagatgagcaaggatctggaggaggtgaaggccaaggtgcagccgtacctggacgacttccagaagaagtggcaggaggagatggagctctaccgccagaaggtggagccgctgcgcgcagagctgcaggagggcgcgcgccagaagctgcacgagctgcaagagaagctgagcccactgggcgaggagatgcgcgaccgcgcgcgcgcccatgtcgacgcgctgcgcacgcatctggcgccgtacagcgacgagctgcgccagcgcttagcggcgcgccttgaggctctcaaggagaacggcggggcccgcctggccgagtaccacgccaaggccaccgagcatctgagcacgctcagcgagaaggccaagccggcgctcgaggacctgcgccaaggcctgctgccggtgctggagagcttcaaggtcagcttcctgagcgctctggaagagtacactaagaagcttaacacccagtga

SEQ ID NO: 19 - DNA, 774 bp, synthetic construct
  source 1..774: mol_type = other DNA
catcaccatcaccatcacggcctggtgccgcgcggcagcatcgatgatccgccgcagagtccatgggatcgcgtgaaggacctggccactgtgtacgtggatgtgctcaaagacagcggcagagactatgtgtctcagtttgaaggatccgccttgggcaaacaattgaaccttaagctgctggacaactgggacagcgtgacgtccaccttcagcaagctgcgcgaacagctcggccctgtgacccaggaattctgggataacctggaaaaggagacagagggcctgcgccaggagatgagcaaggatctggaggaggtgaaggccaaggtgcagccgtacctggacgacttccagaagaagtggcaggaggagatggagctctaccgccagaaggtggagccgctgcgcgcagagctgcaggagggcgcgcgccagaagctgcacgagctgcaagagaagctgagcccactgggcgaggagatgcgcgaccgcgcgcgcgcccatgtcgacgcgctgcgcacgcatctggcgccgtacagcgacgagctgcgccagcgcttagcggcgcgccttgaggctctcaaggagaacggcggggcccgcctggccgagtaccacgccaaggccaccgagcatctgagcacgctcagcgagaaggccaagccggcgctcgaggacctgcgccaaggcctgctgccggtgctggagagcttcaaggtcagcttcctgagcgctctggaagagtacactaagaagcttaacacccagtga

SEQ ID NO: 20 - DNA, 756 bp, synthetic construct
  source 1..756: mol_type = other DNA
ggcctggtgccgcgcggctgcatcgatgatccgccgcagagtccatgggatcgcgtgaaggacctggccactgtgtacgtggatgtgctcaaagacagcggcagagactatgtgtctcagtttgaaggatccgccttgggcaaacaattgaaccttaagctgctggacaactgggacagcgtgacgtccaccttcagcaagctgcgcgaacagctcggccctgtgacccaggaattctgggataacctggaaaaggagacagagggcctgcgccaggagatgagcaaggatctggaggaggtgaaggccaaggtgcagccgtacctggacgacttccagaagaagtggcaggaggagatggagctctaccgccagaaggtggagccgctgcgcgcagagctgcaggagggcgcgcgccagaagctgcacgagctgcaagagaagctgagcccactgggcgaggagatgcgcgaccgcgcgcgcgcccatgtcgacgcgctgcgcacgcatctggcgccgtacagcgacgagctgcgccagcgcttagcggcgcgccttgaggctctcaaggagaacggcggggcccgcctggccgagtaccacgccaaggccaccgagcatctgagcacgctcagcgagaaggccaagccggcgctcgaggacctgcgccaaggcctgctgccggtgctggagagcttcaaggtcagcttcctgagcgctctggaagagtacactaagaagcttaacacccagtga

SEQ ID NO: 21 - DNA, 756 bp, synthetic construct
  source 1..756: mol_type = other DNA
ggcctggtgccgcgcggcagcatcgatgatccgccgcagagtccatgggatcgcgtgaaggacctggccactgtgtacgtggatgtgctcaaagacagcggcagagactatgtgtctcagtttgaaggatccgccttgggcaaacaattgaaccttaagctgctggacaactgggacagcgtgacgtccaccttcagcaagctgcgcgaacagctcggccctgtgacccaggaattctgggataacctggaaaaggagacagagggcctgcgccaggagatgagcaaggatctggaggaggtgaaggccaaggtgcagccgtacctggacgacttccagaagaagtggcaggaggagatggagctctaccgccagaaggtggagccgctgcgcgcagagctgcaggagggcgcgcgccagaagctgcacgagctgcaagagaagctgtgcccactgggcgaggagatgcgcgaccgcgcgcgcgcccatgtcgacgcgctgcgcacgcatctggcgccgtacagcgacgagctgcgccagcgcttagcggcgcgccttgaggctctcaaggagaacggcggggcccgcctggccgagtaccacgccaaggccaccgagcatctgagcacgctcagcgagaaggccaagccggcgctcgaggacctgcgccaaggcctgctgccggtgctggagagcttcaaggtcagcttcctgagcgctctggaagagtacactaagaagcttaacacccagtga

SEQ ID NO: 22 - protein, 12 aa, synthetic construct
GGSGGSGGSGGS

(19) United States
(12) Patent Application Publication
(10) Pub. No.: US 2026/0041638 A1
(43) Pub. Date: Feb. 12, 2026

(54) MODIFIED APOLIPOPROTEINS WITH A TARGETING BODY FOR LIPID NANOPARTICLES

(71) Applicant: Bio-TRIP B.V., Eindhoven (NL)

(72) Inventors: Roy VAN DER MEEL, Eindhoven (NL); Willem J.M. MULDER, Eindhoven (NL); Maarten Merkx, Eindhoven (NL); David Pepijn Schrijver, Amsterdam (NL); Anne De Dreu, Helmond (NL); Ayla Martine Hokke, Waalre (NL); Koen De Bruin, Utrecht (NL)

(21) Appl. No.: 18/865,898

(22) PCT Filed: May 24, 2023
(86) PCT No.: PCT/EP2023/063967

§ 371 Date: Nov. 14, 2024

(30) Foreign Application Priority Data
May 24, 2022 (NL) 2031977
Sep. 23, 2022 (WO) PCT/EP2022/076593
Mar. 29, 2023 (EP) 231652736

(51) Int. Cl. A61K 9/1275; A61K 9/1272; C07K 14/775; C07K 16/28; C12N 15/88

(52) U.S. Cl. CPC A61K 9/1275; A61K 9/1272; C07K 14/775; C07K 16/2815; C12N 15/88; C07K 2317/569; C07K 2319/00

(57) ABSTRACT

The invention relates to modified apolipoprotein with a targeting body. The targeting body may for example be an antibody or antigen binding fragment that allows targeting of e.g. a specific cell, tissue or organ. The modified apolipoprotein can be used as a carrier for a payload as such or when incorporated in a lipid nanoparticle. The modified apolipoprotein finds use in the treatment or prevention of diseases, or targeting a pay load to a specific target site.

### FIELD OF THE INVENTION

The invention relates to the field of modified apolipoproteins that find use in the treatment or prevention of disorders. More specifically the invention relates to apolipoprotein, apolipoprotein derivative, apolipoprotein mimetic or apolipoprotein mimetic derivative with a targeting body. The invention further relates to lipid nanoparticles comprising the modified apolipoproteins and methods of making such. Lastly the invention relates to methods of treatment using the modified apolipoproteins or lipid nanoparticles comprising such.

### INTRODUCTION

Many promising therapeutics suffer from the fact that they either do not, or poorly, reach the intended target site, or present undesired off-target effects. Additionally, many therapies may trigger unwanted immune responses, resulting in degradation of the therapeutic or undesired inflammatory reactions. Therefore, there is a continuous need to improve or provide alternative ways for targeting of therapeutics and to evade immune responses while delivering the therapeutic to the intended target.

These, among other, problems are addressed by the products and methods as defined in the appended claims.

### SUMMARY OF THE INVENTION

The present invention is based on the inventors' finding that apolipoproteins, apolipoprotein derivatives, apolipoprotein mimetics or apolipoprotein mimetic derivatives may be modified to target specific cells, tissues or organs, by attaching them to a targeting body, wherein the targeting body is capable of binding a molecule on the cell surface of a target cell, and that such modified apolipoproteins can be used as a carrier for therapeutic agents, either when being used as such, meaning not as part of a lipid nanoparticle, or when as part of a lipid nanoparticle, preferably a spherical lipid nanoparticle.

Moreover, present inventors unexpectedly found that attaching a targeting body to an apolipoprotein, apolipoprotein derivative, an apolipoprotein mimetic or apolipoprotein mimetic derivative allows to easily incorporate said targeting body in a lipid nanoparticle comprising an outer layer and a core, preferably a spherical lipid nanoparticle, and to expose said targeting body to the environment surrounding said lipid nanoparticle. In this way the modified apolipoprotein can be targeted to cells, tissues or organs it would otherwise not or insufficiently reach, or it could be used to reduce off-target effects.

In a first aspect, the invention relates to a modified apolipoprotein comprising an apolipoprotein, apolipoprotein derivative, apolipoprotein mimetic or apolipoprotein mimetic derivative attached to a targeting body, wherein the targeting body is a molecule capable of binding a molecule on the cell surface of a target cell.

In a second aspect the invention relates to a lipid nanoparticle comprising an outer layer and a core, wherein the outer layer comprises:
a phospholipid;
a sterol; and
the modified apolipoprotein as disclosed herein; and wherein the core comprises at least one core component selected from:
a lipid, a cationic lipid, or a polyvalent molecule.

In a third aspect the invention relates to a lipid nanoparticle comprising an outer layer and a core, wherein the outer layer comprises:
a phospholipid;
a sterol; and
the modified apolipoprotein as disclosed herein; and
   wherein the core comprises a nucleic acid and a cationic or ionizable cationic lipid.

In a fourth aspect the invention relates to a method of manufacturing a lipid nanoparticle as disclosed herein, the method comprising the steps of:
a1) expressing and isolating a modified apolipoprotein as disclosed herein to obtain an isolated modified apolipoprotein; and/or
a2) chemically conjugating a targeting body to an apolipoprotein or apolipoprotein mimetic to obtain a modified apolipoprotein and isolating the modified apolipoprotein;
b) combining the isolated modified apolipoprotein obtained in step a1 and/or step
a2 with phospholipids, sterols and optionally lipids to obtain a lipid nanoparticle.

In a fifth aspect the invention relates to a method for producing a lipid nanoparticle, comprising the step of:
a) rapid mixing of lipid components in organic solvent with a nucleic acid in an aqueous buffer to produce lipid nanoparticles, wherein the lipid components comprise a phospholipid, a sterol, a cationic lipid or ionizable cationic lipid, wherein the aqueous buffer has a pH of 5.0 or lower; and
b) rapid mixing of the lipid nanoparticles with one or more modified apolipoprotein as taught herein;
to produce the lipid nanoparticle at a pH between 5.5 and 8.0, preferably pH between 6.0 and 8.0.

In a sixth aspect the invention relates to a modified apolipoprotein according to the first aspect or the lipid nanoparticle according to the second or third aspect, or the lipid nanoparticle obtained or obtainable by the method of the fourth or fifth aspect of the invention for use as a medicament.

In a seventh aspect, the invention relates to a modified apolipoprotein according to the first aspect or the lipid nanoparticle according to the second or third aspect, or the lipid nanoparticle obtained or obtainable by the method of the fourth or fifth aspect of the invention for use in the treatment or prevention of an immune related disorder such as transplantation rejection, graft-versus-host disease (GVH), atherosclerosis, infection, inflammation, auto-immunity, allergy, cancer, a genetic disorder, a metabolic disorder, a neurological disorder or tissue trauma.

In a eighth aspect the invention relates to the use of a modified apolipoprotein according to the second or third aspect, or the lipid nanoparticle obtained or obtainable by the method of the fourth or fifth aspect of the invention in delivering a compound to a target, preferably wherein the target is a cell, tissue, and/or organ, even more preferably wherein the target is a lymphoid cell, a myeloid cell, a tumor cell, an endothelial cell, a hematopoietic stem and progenitor cell (HSPC), a hematopoietic stem cell (HSC), a multipotent progenitor (MPP), a common myeloid progenitor cell (CMP), or wherein the targeting body binds a bacterial, viral, fungal or parasitic protein or antigen, preferably wherein the lymphoid or myeloid cell is selected from: a monocyte, a macrophage, an M1-like macrophage, an M2-like macrophage, an eosinophil, a basophil, a mast cell, an NK cell, a B cell, a plasma cell, a regulatory T cell, a hematopoietic stem cell, a T helper cell such as Th1, Th2, Th17 or Th22, a dendritic cell, such as a plasmacytoid dendritic cell, a conventional DC 1 or a conventional DC 2, or a tumor-associated macrophage.

In a ninth aspect the invention relates to an in vitro or ex vivo method for introducing a nucleic acid in a cell, the method comprising contacting the lipid nanoparticle as disclosed herein, or the lipid nanoparticle obtained or obtainable by the method as disclosed herein, with a cell.

In a tenth aspect the invention relates to an in vivo method for introducing a nucleic acid in a cell, the method comprising contacting the lipid nanoparticle as disclosed herein, or the lipid nanoparticle obtained or obtainable by the method as disclosed herein, with a cell.

In an eleventh aspect the invention relates to a method for the in vivo delivery of a nucleic acid, the method comprising administering the lipid nanoparticle as disclosed herein, or the lipid nanoparticle obtained or obtainable by the method as disclosed herein, to a subject.

In a twelfth aspect the invention relates to a method for treating a disease or disorder in a subject in need thereof, the method comprising administering a therapeutically effective amount of the lipid nanoparticle as disclosed herein, or the lipid nanoparticle obtained or obtainable by the method as disclosed herein, to the subject.

### BRIEF DESCRIPTION OF THE FIGURES

FIG. 1 depicts an embodiment of the invention, where a modified apolipoprotein (in this case an apolipoprotein-rerouter fusion protein and more particularly an apolipoprotein-nanobody (VHH) fusion protein) is included in a lipid nanoparticle, such as a spherical lipid nanoparticle. The apolipoprotein nanoparticle's (aNP) main constituents are lipid (e.g. phospholipid), sterol (e.g. cholesterol) and (modified) apolipoprotein.

FIG. 2 is a schematic overview of apolipoprotein (e.g. apolipoprotein A1 (apoA1))-rerouter fusion protein nanoparticle technology. (A) Targeting or rerouting protein is a molecule capable of binding a molecule (on the cell surface) of a target cell and can include an antibody (fragment), protein ligand, peptide, peptidomimetic, or sugar polymer. (B) Apolipoprotein-rerouter fusion protein is a modified apolipoprotein comprising an apolipoprotein, apolipoprotein derivative, apolipoprotein mimetic or apolipoprotein mimetic derivative attached to a targeting body, and more particularly to a rerouting peptide or protein. (C) Apoliprotein-rerouter fusion proteins are readily and stably incorporated in lipid nanoparticles, such as spherical lipid nanoparticles, comprising (phospho) lipids, sterols. In another example, apolipoprotein-rerouter fusion proteins are readily and stably incorporated in spherical lipid nanoparticles comprising (phospho) lipids, sterols, ionizable cationic lipids, and therapeutic payloads including nucleic acids such as small interfering RNA or messenger RNA.

FIG. 3 shows the expression of VHHCD8-apolipoprotein A1 (apoA1) fusion protein in Clearcoli cells. Minor protein contaminants are present after IMAC purification [lane E1]. The most prominent band corresponds to the fusion protein with a molecular weight of 43.3 kDa (rectangle).

FIG. 4 shows the mean fluorescence intensity (MFI) of fluorescently labelled VHHCD8-apoA1 and apoA1 in mouse splenocytes (upper panel: CD3+ T cells from splenocytes; lower panel: all cells from the spleen).

FIG. 5 shows the mean fluorescence intensity (MFI) of apolipoprotein nanoparticles (aNPs) formulated with VHHCD8-apoA1 and apoA1 and comprising a fluorescent dye in the lipid structure of the particle in mouse splenocytes.

FIG. 6 shows the successful formulation of spherical apolipoprotein nanoparticles (aNPs) comprising VHHCD8-apoA1 fusion proteins using cryogenic transmission electron microscopy (cryo-TEM) (bottom panel) and the analysis of nanoparticle size and poly dispersity index (PDI) for 14 days using dynamic light scattering (DLS) (top panel).

FIG. 7 In vivo biodistribution study setup for of spherical apolipoprotein nanoparticles (aNPs) comprising VHHCD8-apoA1 fusion proteins.

FIG. 8 Cell association of VHHCD8-apolipoprotein A1 fusion protein nanoparticles in vivo. Flow cytometry analysis of fluorescent apolipoprotein A1 protein nanoparticle (apoA1) and VHHCD8-apolipoprotein A1 fusion protein nanoparticle (VHHCD8) myeloid cell (A), CD8 T cell (B), CD4 T cell (C), CD3 T cell (D) association in bone marrow (BM), spleen, and blood following intravenous administration. MFI: 15 mean fluorescence intensity.

FIG. 9 Cell association of VHHCD8-apolipoprotein A1 fusion protein nanoparticles in vivo. Flow cytometry analysis of fluorescent apolipoprotein A1 protein nanoparticle (apoA1) and VHHCD8-apolipoprotein A1 fusion protein nanoparticle (VHHCD8) CD8 T cell, CD4 T cell and CD3 T cell association in lymph nodes following 20 intravenous administration. MFI: mean fluorescence intensity.

FIG. 10 Cell association of VHHCD8-apolipoprotein A1 fusion protein nanoparticles in vivo. Flow cytometry analysis of fluorescent apolipoprotein A1 protein nanoparticle (apoA1) and VHHCD8-apolipoprotein A1 fusion protein nanoparticle (VHHCD8) cell association in bone marrow (BM), blood, spleen, and lymph nodes 25 following intravenous administration.

FIG. 11 FRET assay of lipid nanoparticles containing VHHCD8-apoliprotein A1 fusion proteins and/or IL-2-apolipoprotein A1 fusion proteins.

FIG. 12 Functional reporter gene expression in mouse splenocytes ex vivo. Flow cytometry analysis of mouse splenocytes incubated with apoA1 aNP, VHHCD8-apoA1 aNP, and VHHGFP-apoA1 aNP containing mRNA encoding for fluorescent mCherry protein (left panel: CD3+ CD4-mCherry+ T cells, right panel: CD11b+mCherry+myeloid cells).

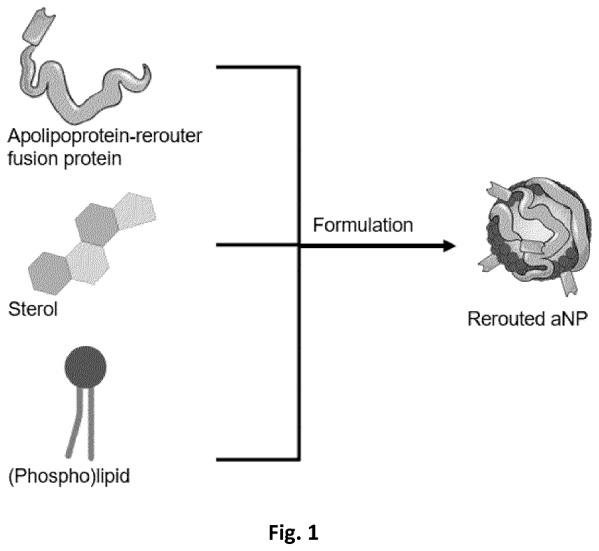

### Detailed description of the invention

For purposes of the present invention, the following terms are defined below.

As used herein, the singular form terms “A,” “an,” and “the” include plural referents unless the content clearly dictates otherwise. Thus, for example, reference to “a cell” includes a combination of two or more cells, and the like.

As used herein, the term “and/or” refers to a situation wherein one or more of the stated cases may occur, alone or in combination with at least one of the stated cases, up to with all of the stated cases.

As used herein, the term “antigen” refers to a substance to which a binding portion of an antibody may bind. The specific immunoreactive sites within the antigen are known as “epitopes” (or antigenic determinants). A target for an antibody, or antigen-binding portion thereof, may comprise an antigen, such as is defined herein.

As used herein, the term “at least” a particular value means that particular value or more. For example, “at least 2” is understood to be the same as “2 or more” i.e., 2, 3, 4, 5, 6, 7, 8, 9, 10, 11, 12, 13, 14, 15, . . . , etc. As used herein, the term “at most” a particular value means that particular value or less. For example, “at most 5” is understood to be the same as “5 or less” i.e., 5, 4, 3, . . . 10,−11, etc.

As used herein, the word “comprise” or variations thereof such as “comprises” or “comprising” will be understood to include a stated element, integer or step, or group of elements, integers or steps, but not to exclude any other element, integer or steps, or groups of elements, integers or steps. The verb “comprising” includes the verbs “essentially consisting of” and “consisting of”.

As used herein, the term “conventional techniques” refers to a situation wherein the methods of carrying out the conventional techniques used in methods of the invention will be evident to the skilled worker. The practice of conventional techniques in molecular biology, biochemistry, computational chemistry, cell culture, recombinant DNA, bioinformatics, genomics, sequencing and related fields are well-known to those of skill in the art and are discussed, for example, in the following literature references: Sambrook et al., Molecular Cloning. A Laboratory Manual, 2nd Edition, Cold Spring Harbor Laboratory Press, Cold Spring Harbor, N. Y., 1989; Ausubel et al., Current Protocols in Molecular Biology, John Wiley & Sons, New York, 1987 and periodic updates; and the series Methods in Enzymology, Academic Press, San Diego.

As used herein, the term “identity” refers to a measure of the identity of nucleotide sequences or amino acid sequences. In general, the sequences are aligned so that the highest order match is obtained. “Identity” per se has an art-recognized meaning and can be calculated using published techniques. See, e.g.: (Computational Molecular Biology, Lesk, A. M., ED., Oxford University Press, New York, 1988; Biocomputing: Informatics And Genome Projects, Smith, D. W., ED., Academic Press, New York, 1993; Computer Analysis Of Sequence Data, Part I, Griffin, A. M., And Griffin, H. G., EDS., Humana Press, New Jersey, 1994; Sequence Analysis In Molecular Biology, Von Heinje, G., Academic Press, 1987; and Sequence Analysis Primer; Gribskov, M. and Devereux, J., eds., M Stockton Press, New York, 1991). While there exist a number of methods to measure identity between two nucleotide sequences or amino acid sequences, the term “identity” is well known to skilled artisans (Carillo, H., and Lipton, D., SIAM J. Applied Math (1988) 48:1073). Methods commonly employed to determine identity or similarity between two sequences include, but are not limited to, those disclosed in Guide To Huge Computers, Martin J. Bishop, ed., Academic Press, San Diego, 1994, and Carillo, H., and Lipton, D., Siam J. Applied Math (1988) 48:1073. Methods to determine identity and similarity are codified in computer programs. Preferred computer program methods to determine identity and similarity between two sequences include, but are not limited to, GCS program package (Devereux, J., et al., Nucleic Acids Research (1984) 12 (1): 387), BLASTP, BLASTN, FASTA (Atschul, S. F. et al., J. Molec. Biol. (1990) 215:403).

As an illustration, by a polynucleotide having a nucleotide sequence having at least, for example, 95% “identity” to a reference nucleotide sequence encoding a polypeptide of a certain sequence, it is intended that the nucleotide sequence of the polynucleotide is identical to the reference sequence except that the polynucleotide sequence may include up to five point mutations per each 100 nucleotides of the reference amino acid sequence. In other words, to obtain a polynucleotide having a nucleotide sequence at least 95% identical to a reference nucleotide sequence, up to 5% of the nucleotides in the reference sequence may be deleted and/or substituted with another nucleotide, and/or a number of nucleotides up to 5% of the total nucleotides in the reference sequence may be inserted into the reference sequence. These mutations of the reference sequence may occur at the 5′ or 3′ terminal positions of the reference nucleotide sequence, or anywhere between those terminal positions, interspersed either individually among nucleotides in the reference sequence or in one or more contiguous groups within the reference sequence.

Similarly, by a polypeptide having an amino acid sequence having at least, for example, 95% “identity” to a reference amino acid sequence of SEQ ID NO: X is intended that the amino acid sequence of the polypeptide is identical to the reference sequence except that the amino acid sequence may include up to five amino acid alterations per each 100 amino acids of the reference amino acid of SEQ ID NO: X. In other words, to obtain a polypeptide having an amino acid sequence at least 95% identical to a reference amino acid sequence, up to 5% of the amino acid residues in the reference sequence may be deleted or substituted with another amino acid, or a number of amino acids up to 5% of the total amino acid residues in the reference sequence may be inserted into the reference sequence. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As used herein, the term “in vitro” refers to experimentation or measurements conducted using components of an organism that have been isolated from their natural conditions.

As used herein, the term “ex vivo” refers to experimentation or measurements done in or on tissue from an organism in an external environment with minimal alteration of natural condition.

As used herein, the term “nucleic acid”, “nucleic acid molecule” and “polynucleotide” is intended to include DNA molecules and RNA molecules. A nucleic acid (molecule) may be single-stranded or double-stranded, but preferably is double-stranded DNA or single- or double stranded RNA, more preferably single- or double stranded RNA.

The terms “nucleic acid”, “nucleic acid molecule” and “polynucleotide” are well understood in the art. By means of further guidance, the terms typically refer to a polymer (preferably a linear polymer) of any length composed essentially of nucleoside units. A nucleoside unit commonly includes a heterocyclic base and a sugar group. Heterocyclic bases may include inter alia purine and pyrimidine bases such as adenine (A), guanine (G), cytosine (C), thymine (T) and uracil (U), which are widespread in naturally-occurring nucleic acids, other naturally-occurring bases (e.g., xanthine, inosine, hypoxanthine), as well as chemically or biochemically modified (e.g., methylated), non-natural or derivatised bases. Exemplary modified nucleobases include, without limitation, 5-substituted pyrimidines, 6-azapyrimidines and N-2, N-6 and O-6 substituted purines, including 2-aminopropyladenine, 5-propynyluracil and 5-propynylcytosine. In particular, 5-methylcytosine substitutions have been shown to increase nucleic acid duplex stability. Sugar groups may include inter alia pentose (pentofuranose) groups such as preferably ribose and/or 2-deoxyribose common in naturally-occurring nucleic acids, or arabinose, 2-deoxyarabinose, threose or hexose sugar groups, as well as modified or substituted sugar groups (such as, without limitation, 2′-O-alkylated, e.g., 2′-O-methylated or 2′-O-ethylated sugars such as ribose; 2′-O-alkyloxyalkylated, e.g., 2′-O-methoxyethylated sugars such as ribose; or 2′-0,4′-C-alkylene-linked, e.g., 2′-0,4′-C-methylene-linked or 2′-0,4′-C-ethylene-linked sugars such as ribose; 2′-fluoro-arabinose, etc.). Nucleoside units may be linked to one another by any one of numerous known inter-nucleoside linkages, including inter alia phosphodiester linkages common in naturally-occurring nucleic acids, and further modified phosphate- or phosphonate-based linkages such as phosphorothioate, alkyl phosphorothioate such as methyl phosphorothioate, phosphorodithioate, alkylphosphonate such as methylphosphonate, alkylphosphonothioate, phosphotriester such as alkylphosphotriester, phosphoramidate, phosphoropiperazidate, phosphoromorpholidate, bridged phosphoramidate, bridged methylene phosphonate, bridged phosphorothioate; and further siloxane, carbonate, sulfamate, carboalkoxy, acetamidate, carbamate such as 3′-N-carbamate, morpholino, borano, thioether, 3′-thioacetal, and sulfone internucleoside linkages. Preferably, inter-nucleoside linkages may be phosphate-based linkages including modified phosphate-based linkages, such as more preferably phosphodiester, phosphorothioate or phosphorodithioate linkages or combinations thereof. The term “nucleic acid” also encompasses any other nucleobase containing polymers such as nucleic acid mimetics, including, without limitation, peptide nucleic acids (PNA), peptide nucleic acids with phosphate groups (PHONA), locked nucleic acids (LNA), morpholino phosphorodiamidate-backbone nucleic acids (PMO), cyclohexene nucleic acids (CeNA), tricyclo-DNA (tcDNA), and nucleic acids having backbone sections with alkyl linkers or amino linkers (see, e.g., Kurreck 2003 (Eur J Biochem 270:1628-1644)). “Alkyl” as used in this context particularly encompasses lower hydrocarbon moieties, e.g., C₁-C₄ linear or branched, saturated or unsaturated hydrocarbon, such as methyl, ethyl, ethenyl, propyl, 1-propenyl, 2-propenyl, and isopropyl.

Nucleic acids as intended herein may include naturally occurring nucleosides, modified nucleosides or mixtures thereof. A modified nucleoside may include a modified heterocyclic base, a modified sugar moiety, a modified inter-nucleoside linkage or a combination thereof. The term “nucleic acid” further preferably encompasses DNA, RNA and DNA/RNA hybrid molecules, specifically including hnRNA, pre-mRNA, mRNA, cDNA, genomic DNA, amplification products, oligonucleotides, and synthetic (e.g., chemically synthesised) DNA, RNA or DNA/RNA hybrids. A nucleic acid can be naturally occurring, e.g., present in or isolated from nature, can be recombinant, i.e., produced by recombinant DNA technology, and/or can be, partly or entirely, chemically or biochemically synthesised. A “nucleic acid” can be double-stranded, partly double stranded, or single-stranded. Where single-stranded, the nucleic acid can be the sense strand or the antisense strand. In addition, nucleic acid can be circular or linear.

As used herein, the terms “sequence” when referring to nucleotides, or “nucleic acid sequence”, “nucleotide sequence” or “polynucleotide sequence” refer to the order of nucleotides of, or within, a nucleic acid and/or polynucleotide. Within the context of the current invention a first nucleic acid sequence may be comprised within or overlap with a further nucleic acid sequence.

As used herein, the term “subject” or “individual” or “animal” or “patient” or “mammal,” used interchangeably, refer to any subject, particularly a mammalian subject, for whom diagnosis, prognosis, or therapy is desired. Mammalian subjects include humans, domestic animals, farm animals, and zoo-, sports-, or pet-animals such as dogs, cats, guinea pigs, rabbits, rats, mice, horses, cattle, cows, bears, and so on. As defined herein a subject may be alive or dead. Samples can be taken from a subject post-mortem, i.e. after death, and/or samples can be taken from a living subject.

As used herein, terms “treatment”, “treating”, “palliating”, “alleviating” or “ameliorating”, used interchangeably, refer to an approach for obtaining beneficial or desired results including, but not limited to, therapeutic benefit. By therapeutic benefit is meant eradication or amelioration or reduction (or delay) of progress of the underlying disease being treated. Also, a therapeutic benefit is achieved with the eradication or amelioration or reduction (or delay) of progress of one or more of the physiological symptoms associated with the underlying disease such that an improvement or slowing down or reduction of decline is observed in the patient, notwithstanding that the patient can still be afflicted with the underlying disease.

As used herein the term “vector” refers to a nucleic acid molecule capable of transporting another nucleic acid to which the nucleic acid molecule capable of transporting has been linked. One type of vector is a “plasmid”, which refers to a circular double stranded DNA loop into which additional DNA segments may be ligated. The term “vector” may also refer to the viral particle (i.e. viral vector) which contains the nucleic acid of interest.

A portion of this invention contains material that is subject to copyright protection (such as, but not limited to, diagrams, device photographs, or any other aspects of this submission for which copyright protection is or may be available in any jurisdiction). The copyright owner has no objection to the facsimile reproduction by anyone of the patent document or patent invention, as it appears in the Patent Office patent file or records, but otherwise reserves all copyright rights whatsoever.

Various terms relating to the methods, compositions, uses and other aspects of the present invention are used throughout the specification and claims. Such terms are to be given their ordinary meaning in the art to which the invention relates, unless otherwise indicated. Other specifically defined terms are to be construed in a manner consistent with the definition as provided herein. The preferred materials and methods are described herein, although any methods and materials similar or equivalent to those described herein can be used in the practice for testing of the present invention.

Unless otherwise defined, all technical and scientific terms used herein have the same meaning as commonly understood by a person of ordinary skill in the art.

The present invention is based on the inventors' finding that apolipoprotein can be modified to target specific cells, tissues, organs or pathogens. The inventors found that modified apolipoproteins that bind a targeting body allow targeting of lipid nanoparticles containing such modified apolipoproteins. As lipid nanoparticles (or modified apolipoproteins) can be loaded with a payload such as a pharmaceutical, this allows the direct targeting of a pharmaceutical to a desired target site. Furthermore, the modified apolipoprotein of the present invention also allows to prepare alipid nanoparticle, wherein the targeting body is exposed to the environment (i.e. aqueous environment) surrounding said apolipoprotein lipid nanoparticle, as a result.

Therefore, in a first aspect, the invention relates to a modified apolipoprotein comprising an apolipoprotein, apolipoprotein derivative, apolipoprotein mimetic or apolipoprotein mimetic derivative attached to a targeting body, wherein the targeting body is a molecule capable of binding a molecule on the cell surface of a target cell.

The modified apolipoproteins may be used as such, meaning not as part of a lipid nanoparticle. In such way the modified apolipoproteins may serve as a carrier to deliver a payload (e.g. a therapeutic) to a target site. A payload could for example be bound to the modified apolipoproteins, for example by a covalent bond. The targeting body can be used to target the modified apolipoproteins to a specific site, such as a cell, tissue, organ or pathogen.

When used herein, the term attached when referring to the modified apolipoprotein may refer to a stable or semi-stable association, for example but not limited to a covalent bond, a hydrogen bond or van der Waals force, or combinations thereof, such as but not limited to a peptide bond, a sulfur bond, a protein-protein interaction or a protein ligand interaction.

### Targeting Body

When used herein a targeting body refers to a molecule that allows, when attached to a protein such as an apolipoprotein, the protein to bind to a different target than it would bind when the protein was not fused to the targeting body (or in other words, a different target than that to which it would have innately bound), and/or to bind to its intended target with a higher affinity.

In particular embodiments, the targeting body may allow to bind a different target, such as a particular subset of target cells, including binding to a particular subset of a set of cells which would normally be bound by the modified apolipoprotein. For example, apoA1 is known to bind receptors on myeloid cells, thus the targeting body may be used to bind a particular subtype of myeloid cells. For example, as described elsewhere herein, SIRPalpha as a targeting body may allow to target immunosuppressive macrophages. Non limiting examples of targeting bodies are an antibody or antigen binding fragment thereof or an antibody fragment, a rerouting peptide or a rerouting protein, preferably wherein the rerouting peptide or rerouting protein is a ligand of a receptor present on the target.

Therefore in an embodiment the targeting body is selected from:
an antibody or antigen binding fragment of an antibody,
a protein ligand, a protein binding domain, or a protein binding fragment thereof,
a peptide,
a peptidometic, or
a sugar polymer.

When used herein the term target refers to the object the modified apolipoprotein or targeting body preferentially binds to. A target may refer to a receptor or cell surface molecule such as a protein or proteoglycan, a lipid, a phospholipid, a sugar, a sugar polymer, a cell, a cell type, a tissue or tissue type or an organ.

It is appreciated that apolipoproteins bind to specific ligands. For example, it is thought that the different apolipoproteins found in different lipoproteins (e.g. HDL, LDL, VLDL, etc.) are responsible for differences in targeting and binding and thus function of the lipoproteins. Without wishing to be bound by theory, it is thought that part of the apolipoprotein has an amphipathic nature and is responsible, together with phospholipids and/or sterols to bind lipids in an aqueous environment, while different parts of the molecule are responsible for interacting with other molecules, e.g. binding to protein receptors. It is further assumed that apolipoproteins may circulate also as proteins, meaning not as lipoproteins. Therefore, the possibility to modify binding affinity of the apolipoprotein (by adding a targeting body) provides interesting opportunities, as it allows to fine-tune targeting or binding of the apolipoprotein. Several applications are envisioned for such modified apolipoproteins:

First the targeting body may simply be used to reroute the apolipoprotein or lipoprotein, for example to make changes in the lipid homeostasis. For example, one could envision that LDL or HDL values in the blood of a subject could be altered by using apolipoprotein combined with a targeting body. This could potentially be exploited for treatment of lipid disorders such as high blood cholesterol levels.

Second it may be used to reroute a lipoprotein (lipid nanoparticle) with a payload to a predetermined target. Lipoproteins or lipid nanoparticles pose interesting methods of carrying a payload such as a pharmaceutical compound. It allows delivery of lipophilic compounds through blood, as the compound may be dissolved in the lipid core of a lipoprotein/lipid nanoparticle. An additional advantage is that the lipoprotein consists of naturally occurring compounds and thus is seen as native by the immune system, avoiding triggering an immune response by the pharmaceutical compound.

Third, it may be further combined with a payload, and as such allow the targeting of the payload. As described above, apolipoprotein can essentially serve as a carrier for a payload. Advantages are that it greatly reduces clearing of the payload, making it feasible to use payloads that are easily cleared from the blood in a therapeutic setting (e.g. cytokines). One issue that might arise is that payload/apolipoprotein combination does not arrive at the intended target for the payload (i.e. the site, cell, tissue or organ where it is intended to exert its effect). This is solved by further including the targeting body.

Data generated by the inventors suggests that by using a targeting body the modified apolipoprotein can successfully be rerouted to a different target. Therefore, in an embodiment, the targeting body is selected from an antibody or an antigen binding fragment thereof, a rerouting peptide or a rerouting protein, preferably wherein the rerouting peptide or rerouting protein is a ligand of a receptor present on the target. In an embodiment the targeting body may be an antibody or an antigen binding fragment thereof. It is envisioned that any type of antigen binding molecule can in principle be used as a targeting body in the modified apolipoprotein according to the invention.

In particular embodiments, the targeting body binds to an antigen which is highly expressed or exclusively present on myeloid cells. Suitable targets are known to the skilled person, non-limiting examples are CD11b, CD11c, CD14 or co-stimulatory molecules such as CD80, CD83, CD86, CD40 or HLA-DR. Therefore in an embodiment the targeting body is a myeloid-targeting peptide or protein selected from an antibody or an antigen binding fragment thereof which binds to CD11b, CD11c, CD14, CD80, CD83, CD86, CD40 or HLA-DR.

In other particular embodiments, the targeting body may be a protein ligand which binds to a receptor or factor expressed on a myeloid cell, non-limiting examples being CD40L (CD154) and FC domains, but the skilled person is aware of other suitable ligands or cofactors. Therefore, in an embodiment, the targeting body is a myeloid-targeting peptide or a myeloid-targeting protein wherein the myeloid-targeting protein or myeloid-targeting peptide is selected from CD40L (CD154) and FC domains. In an embodiment the targeting body is an antibody or antigen binding fragment thereof, wherein the antibody or antigen binding fragment thereof is selected from a Fragment antigen-binding region (Fab), a Fab2, a single-chain variable fragment (scFv), a scFv-Fc, a dAb-Fc, a free light chain antibody, a half antibody, a bispecific Fab2, a Fab3, a trispecific Fab3 a diabody, a bispecific diabody, a triabody, a trispecific triabody, a minibody, an lgG, an immunoglobulin new antigen receptor (IgNAR), a monovalent IgG, a VhH, a nanobody, or a variable domain of new antigen receptor (VNAR), or an antigen binding fragment thereof, preferably a camelid or shark VhH or derivative thereof or antigen binding fragment thereof.

The antibody or antigen binding fragment may also be a designed antigen binding protein such as but not limited to affibodies, FN3 domains, DARPins or de novo designed protein receptors. It is appreciated that antibodies or antigen binding fragments thereof with a lower molecular weight are preferred due to their reduced size, therefore in a preferred embodiment the antibody or antigen binding fragment thereof is a Fab, scFv, single domain antibody, VIH or VNAR.

In an embodiment the targeting body may be a rerouting peptide. Non limiting examples of rerouting peptides are receptor binding peptides, and/or ligand mimicking peptides. Therefore, in an embodiment, the rerouting peptide is selected from programmed cell death protein 1 (PD1) or signal-regulatory protein alpha (SIRPa). SIRPalpha as a targeting body may allow to target immunosuppressive macrophages. It is however understood that any peptide with binding specificity to a cell surface receptor could be used as a targeting body.

In an embodiment the targeting body may be a protein such as a receptor ligand, a receptor, or interacting protein. Alternatively, the targeting body may be part of a protein for example a protein or ligand binding domain. Therefore, in an embodiment, the rerouting protein is for example selected from CD40L or GP120. CD40L can be used to target cells expressing the CD40 receptor. GP120 can be used to bind directly to the CD4 T-Cell co-receptor. It is however understood that any protein with binding specificity to a cell surface receptor could be used as a targeting body.

In an embodiment the targeting body may be a sugar polymer. Sugar polymers could be used to direct an apolipoprotein modified with a sugar polymer to specific site or receptor. For example, apolipoprotein could be modified with mannose to direct the modified apolipoprotein to the mannose receptor, which is generally expressed in macrophages, dendritic cells, and endothelial cells. The skilled person would be aware of other sugar polymers that may be used for similar purposes and which cells could be target with these. When used herein the term sugar polymer refers to any poly saccharide, thus any molecule comprising at least two saccharides.

When used herein, the term lipoprotein refers to a particle, generally a nanoparticle, of at least one apolipoprotein and lipid molecules, dispersed or dissolved in an aqueous environment.

When used herein, the term rerouting refers to targeting the modified apolipoprotein to a different target than it would normally bind to, or reducing the binding to the regular target of the modified apolipoprotein or preventing off-target binding. For example, apolipoprotein a1 (apoA1) is known to bind receptors on myeloid cells, thus the targeting body may be used to bind different cells and reduce binding of myeloid cells (for example, the myeloid cell may be a monocyte, a dendritic cell, a tissue macrophage or a granulocyte).

In an embodiment the targeting body binds a protein on the surface of a lymphoid cell, a myeloid cell, a tumor cell, an endothelial cell, a hematopoietic stem and progenitor cell (HSPC), a hematopoietic stem cell (HSC), a multipotent progenitor (MPP), a common myeloid progenitor cell (CMP), or wherein the targeting body binds a bacterial, viral, fungal or parasitic protein or antigen,
preferably wherein the targeting body binds a protein on the surface of a lymphoid cell, a myeloid cell, a tumor cell, or wherein the targeting body binds a bacterial, viral, fungal or parasitic protein or antigen,
preferably wherein the lymphoid or myeloid cell is selected from a monocyte, a macrophage, an M1-like macrophage, an M2-like macrophage, an eosinophil, a basophil, a mast cell, an NK cell, a B cell, a plasma cell, a regulatory T cell, a hematopoietic stem cell, a granulocyte, a T helper cell such as Th1, Th2, Th17 or Th22, a dendritic cell, such as a plasmacytoid dendritic cell, a conventional DC 1 or a conventional DC 2, or a tumor-associated macrophage,
more preferably wherein the lymphoid or myeloid cell is selected from a monocyte, a macrophage, an M1-like macrophage, an M2-like macrophage, an eosinophil, a basophil, a mast cell, an NK cell, a B cell, a plasma cell, a regulatory T cell, a hematopoietic stem cell, a T helper cell such as Th1, Th2, Th17 or Th22, a dendritic cell, such as a plasmacytoid dendritic cell, a conventional DC 1 or a conventional DC 2, or a tumor-associated macrophage.

In particular embodiments, the myeloid cell may be a blood cell derived from a progenitor cell for granulocytes, monocytes, erythrocytes or platelets.

In an embodiment the targeting body binds a protein selected from CD1a, CD1b, CD1c, CD1d, CD2, CD3, CD4, CD5, CD6, CD7, CD8, CD9, CD10, CD11a, CD11b, CD11c, CD11d, CDw12, CD13, CD14, CD15, CD15s, CD15u, CD16, CDw17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42a, CD42b, CD42c, CD42d, CD43, CD44, CD45, CD45RO, CD45RA, CD45RB, CD46, CD47, CD48, CD49a, CD49b, CD49c, CD49d, CD49e, CD49f, CD50, CD51, CD52, CD53, CD54, CD55, CD56, CD57, CD58, CD59, CD60a, CD60b, CD60c, CD61, CD62E,

CD62L, CD62P, CD63, CD64, CD65, CD66a, CD66b, CD66c, CD66d, CD66e, CD66f, CD68, CD69, CD70, CD71, CD72, CD73, CD74, CD75, CD75s, CD77, CD79a, CD79B, CD80, CD81, CD82, CD83, CDw84, CD85, CD86, CD87, CD88, CD89, CD90, CD91, CD92, CD93, CD94, CD95, CD96, CD97, CD98, CD99, CD100, CD101, CD102, CD103, CD104, CD105, CD106, CD107a, CD107b, CD108, CD109, CD110, CD111, CD112, CD114, CD115, CD116, CD117, CD118, CD119, CD120a, CD120b, CD121a, CDw121b, CD122, CD123, CD124, CD125, CD126, CD127, CDw128, CD129, CD130, CDw131, CD132, CD133, CD134, CD135, CDw136, CDw137, CD138, CD139, CD140a, CD140b, CD141, CD142, CD143, CD144, CD145, CD146, CD147, CD148, CD150, CD151, CD152, CD153, CD154, CD155, CD156a, CD156b, CD157, CD158, CD158a, CD158b, CD159a, CD160, CD161, CD162, CD162R, CD163, CD164, CD165, CD166, CD167a, CD168, CD169, CD170, CD171, CD172a, CD173, CD174, CD175, CD175s, CD176, CD177, CD178, CD179a, CD179b, CD180, CD183, CD184, CD195, CDw197, CD200, CD201, CD202b, CD203c, CD204, CD205, CD206, CD207 (CLEC4K), CD208, CD209 (CLEC4L), CDw210, CD212, CD213a1, CD213a2, CDw217, CD220, CD221, CD222, CD223, CD224, CD225, CD226, CD227, CD228, CD229, CD230, CD231, CD232, CD233, CD234, CD235a, CD235b, CD236, CD236R, CD238, CD239, CD240CE, CD240D, CD241, CD242, CD243, CD244, CD245, CD246, CD247, CXCR1, CXCR2, CXCR3, CXCR4, CXCR5, CXCR6, CXCR7, CCR1, CCR2, CCR3, CCR4, CCR5, CCR7, CCR8, CCR9, CLEC1A, CLEC1B, CLEC2A, CLEC2B, CLEC3A, CLEC3B, CLEC4A, CLEC4C (CD303), CLEC4D,CLEC4J CLEC4E, CLEC4F, CLEC4G, ASGR1 (CLEC4H1), ASGR2 (CLEC4H2), FCER2 ( ) CLEC4M, CLEC5A, CLEC6A, CLEC7A, OLR1 (CLEC8A), CLEC9A, CLEC10A, CLEC11A, CLEC12A, CLEC12B, CD302 (CLEC13A), LY75 (CLEC13B), PLA2R1 (CLEC13C), MRC1 (CLEC13D), MRC2 (CLEC13E), CLEC14A, CLEC16A, CLEC17A, KLRA1, KLRB1 (CLEC5B), KLRC1, KLRC2, KLRC3, KLRC4, KLRD1, KLRF1 (CLEC5C), KLRG1 (CLEC15A), KLRG2 (CLEC15B), KLRK1, AGC1, ATRNL1, BCAN, CD248, CHODL, CL-K1-la, CL-K1-Ib, CL-K1-Ic, CLECSF5, COLEC10, COLEC11, COLEC12, CSPG3, FCER2, FREM1, HBXBP, LAYN, LOC348174, LOC728276, MAFA, MBL2, MGC34761, MICL, MRC1L1, PAP, PKD1, PKD1L2, PRG2, PRG3, REG1A, REG1B, REG3A, REG3G, REG4, SELE, SELL, SELP, SFTPA1, SFTPA2, SFTPA2B, SFTPD, SRCL, THBD, VCAN, Alphafetoprotein (AFP), Carcinoembryonic antigen (CEA), CA-125, MUC-1, Epithelial tumor antigen (ETA), Tyrosinase, and melanoma-associated antigen (MAGE).
preferably selected from CD14, CD11b, CD357 (GITR), CD193, CD123, CD117, CD56, CD19, CD38, CD25, CD133, CXCR3, CCR3, CD196, CCR10, CD64, CD206, CLEC4C (CD303), CLEC9A, CD1c, CD163, Alphafetoprotein (AFP), Carcinoembryonic antigen (CEA), CA-125, MUC-1, Epithelial tumor antigen (ETA), Tyrosinase, and melanoma-associated antigen (MAGE).

These targets may be used to target specific cell types or subsets of cells. The skilled person is aware which marker may be suitable for which cell type(s), for example the following proteins could be used to target specific cells:

Cells of the innate immune system:
Monocytes: CD14
Macrophages: CD11b
Innate lymphoid cells: CD357 (GITR)
Eosinophils: CD193
Basophils: CD123
Mast cells: CD117
   Cells of the adaptive immune system:
NK cells: CD56
B cells: CD19
Plasma cells: CD38
Regulatory T cells: CD25
   Subtypes of immune cells:
Myeloid progenitor cells:
Hematopoietic stem cells: CD133
T helper cell subsets: Th1: CXCR3, Th2: CCR3, Th17: CD196, Th22: CCR 10
Macrophages polarization states (M1-like or M2-like): M1: CD64, M2: CD206 (mannose-receptor) (e.g. targeted by apolipoprotein bound with nanobody specific for this receptor, or apolipoprotein bound with mannose)
Subtypes of dendritic cells: plasmacytoid dendritic cells: CD303, conventional DC 1: CLEC9A, conventional DC 2: CD1c
Tumor-associated macrophages: CD163 (or targeted with nanobody against tumor antigen).

In particular embodiments, the targeting body is capable of binding to a non-myeloid cell, such as a non-myeloid immune cell, preferably non-myeloid leukocytes, more preferably lymphocytes, more preferably T cells, even more preferably CD8+ T cells.

In particular embodiments, the targeting body is capable of binding to cells that are not myeloid cells, but that may differentiate into myeloid cells, such as a hematopoietic stem and progenitor cell (HSPC), like a hematopoietic stem cell (HSC), a multipotent progenitor (MPP), or a common myeloid progenitor cell (CMP). In particular embodiments, the targeting body is capable of binding to a non-myeloid cell, such as a non-myeloid immune cell or an endothelial cell. Endothelial cells may be targeted by use of a targeting body capable of binding to a surface marker of endothelial cells. For example, endothelial cells may be targeted by use of a targeting body capable of binding to Factor VIII-related antigen such as Factor VIII, a targeting body capable of binding to CD31/PECAM-1 such as CD31, a targeting body capable of binding to Angiotensin-converting enzyme (ACE/CD143) such as angiotensin, a targeting body capable of binding to CD34 such as L-selectin or a targeting body capable of binding to endoglin (CD105).

In particular embodiments, the non-myeloid cell is a lymphocyte, such as a T cell, a B cell or a natural killer (NK) cell. Preferably, the lymphocyte is a T cell, even more preferably a CD8+ T cell.

In particular embodiments, the targeting body is an antibody or antigen binding fragment thereof binding to, e.g. specifically binding, to CD8.

In particular embodiments, if the target cell is a T cell, the targeting body may be an antibody or antigen binding fragment thereof binding, preferably specifically binding, to CD8. For example, the targeting body may be a VHHCD8 as described in Woodham A.W. et al., Nanobody-antigen conjugates elicit HPV-specific antitumor immune responses, Cancer Immunology Research, 2018, Vol. 6, issue 7, and comprising an amino acid sequence as shown in Supplemental Table 1 of said reference.

For example, CD8-targeted apolipoprotein nanoparticles as described herein can also be used to generate chimeric antigen receptor (CAR) T cells in vivo. In particular, the CD8-targeted apolipoprotein nanoparticles can be used to deliver plasmid DNA (pDNA), linear or circular RNA (e.g. linear or circular mRNA), and/or gene editing components encoding instructions to reprogram T cells in vivo. For example, targeted nucleases can be used to introduce a nucleic acid in a cell. For example, the targeted nucleases include, but are not limited to, transcription activator-like effector nuclease (TALEN), zinc finger nuclease (ZNF), clustered regulatory interspaced short palindromic repeats (CRISPR), CRISPR/Cas9, CRISPR/CPFL and combinations thereof, such as for example using single guide RNA or other gene editing components, such as single guide RNA with CRISPR/Cas. In a particular example, pDNA or RNA encoding chimeric antigen receptors (CAR) can be delivered into T cells that enables them to recognize and kill tumor cells with a cognate ligand. In another particular example, pDNA or RNA encoding chimeric antigen receptors (CAR) can be delivered into T cells that enables them to recognize and eliminate pathologic cells such as activated fibroblasts in fibrotic disorders.

CD8-targeted apolipoprotein nanoparticles as described herein can also be used to modify CD8+ T cell function. For example, CD8+ T cell function may be modified through modifying cytokine-cytokine receptor interactions using CD8-targeted apolipoprotein nanoparticles as described to deliver plasmid DNA (pDNA), linear or circular RNA (e.g. linear or circular mRNA), and/or gene editing components as mentioned above encoding instructions for cytokine production in T cells. In a particular example, pDNA or mRNA encoding cytokines such as IFN-γ, IL-2, IL-10, IL-12, IL-15 may be delivered into T cells to enable them to secrete cytokines. This may result in reprogramming an immunosuppressive tumor microenvironment into an anti-tumor phenotype. In another particular example, pDNA or mRNA encoding cytokines receptors such as IFN-γR, IL-2R, IL-12R, or IL-15R may be delivered into T cells. CD8+ T cell function may also be modified through regulating immune checkpoints, e.g. CD8+ T cell function may be modified through downregulating immune checkpoints using CD8-targeted apolipoprotein nanoparticles as described to deliver antisense oligonucleotides, small interfering RNA (SIRNA), mRNA, and/or gene editing components as mentioned above encoding instructions to downregulate immune checkpoints in T cells. In a particular example, siRNA or gene editing components as mentioned above targeting PD1, CTLA4, SHP-2, LAG3, or TIM-3 may b delivered to T cells for downregulation of checkpoint molecules that inhibit cytotoxic T cell function in cancer.

In yet another example, CD8-targeted apolipoprotein nanoparticles as described herein can be used for re-polarization of pathogenic CD8+ T cells in autoimmunity towards a tolerogenic phenotype. In particular, the CD8-targeted apolipoprotein nanoparticles can be used to deliver antisense oligonucleotides, small interfering RNA (siRNA), mRNA or gene editing components as mentioned above encoding instructions to re-polarize pathogenic T cells. In a particular example, mRNA encoding FOXP3 may be delivered into T cells to alter their phenotype into regulatory T cells (T regs). In another particular example, CD8+IL17+ T cells may be re-polarized via delivery of siRNA targeting IL-17 or the RORyt transcription factor.

In an embodiment, the modified apolipoprotein comprises aapoA1 with VHH8CD8.

In an embodiment, the VHHCD8-apoA1 modified apolipoprotein comprises or consists of a polypeptide with an amino acid sequence at least 90%, at least 91%, at least 92%, at least 93%, at least 94%, at least 95%, at least 96%, at least 97%, at least 98%, at least 99% or 100% identical to SEQ ID NO. 7 or 1, or comprises or consists of an amino acid sequence encoded by a nucleic acid with a sequence at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to SEQ ID NO. 8 or 2.

In particular embodiments, if the target cell is a T and/or B cell, the targeting body may be PD1, CD40L or GP120.

apolipoproteins

apolipoproteins are proteins that bind lipids such as triglycerides and cholesterol to form lipoproteins. They transport lipids (and fat-soluble vitamins) in blood, cerebrospinal fluid and lymph. The lipid components of lipoproteins are insoluble in water. However, because of their detergent-like (amphipathic) properties, apolipoproteins and other amphipathic molecules (such as phospholipids) can surround the lipids, creating a lipoprotein particle that is itself water-soluble, and can thus be carried through water-based circulation (i.e., blood, lymph). In addition to stabilizing lipoprotein structure and solubilizing the lipid component, apolipoproteins interact with lipoprotein receptors and lipid transport proteins, thereby participating in lipoprotein uptake and clearance.

In lipid transport, apolipoproteins function as structural components of lipoprotein particles, ligands for cell-surface receptors and lipid transport proteins, and cofactors for enzymes. Different lipoprotein particles contain different classes of apolipoproteins, which influence their function. For example, apolipoprotein A1 (apoA1) is the major structural protein component of high-density lipoproteins (HDL), although it is present in other lipoproteins in smaller amounts, and HDL comprises other apolipoproteins. It is envisioned that the invention is not limited to a particular type of apolipoprotein, therefore in an embodiment, the apolipoprotein, apolipoprotein derivative, apolipoprotein mimetic or apolipoprotein mimetic derivative is selected from apoA1, apoA-1 Milano, apoA2, apoA4, apoA5, apoB, apoB48, apoB100, apoC-I, apoC-II, apoC-III, apoC-IV, apoD, apoE, apoF, apoH, apoL, apoL1, apoL2, apoL3, apoL4, apoL5, apoL6, apoLD1, apoO, apoOL and apoM, or a combination thereof, or a mimetic or derivative thereof,
preferably selected from apoA1, apoA2, apoA4, apoA5, apoB100, apoC-I, apoC-II, apoC-III, apoC-IV, apoD, apoE, apoF, apoH, apoL, and apoM, or a mimetic or derivative thereof,
more preferably selected from apoA1, apoA2, apoA4, apoA5, apoB100, apoC-I, apoC-II, apoC-III, apoC-IV and apoE or a mimetic or derivative thereof,
even more preferably selected from apoA1, apoA4, apoA5, apoB100, apoC-III and apoE or a mimetic or derivative thereof,
most preferably selected from apoA1, apoB100 and apoE or a mimetic or derivative thereof.

In particular embodiments, the apolipoprotein component of the modified apolipoprotein is apoA1 or an apoA1 mutant.

In particular embodiments, the apolipoprotein component may also be an apolipoprotein fragment. Preferably, the apolipoprotein fragment retains the biological activity of the full-length apolipoprotein, such as the ability of the apolipoprotein to integrate into a lipid nanoparticle or to target to the myeloid compartment. In particular embodiments, the apolipoprotein fragment comprises at least the ATP Binding Cassette Subfamily A Member 1 (ABCA1), ATP Binding Cassette Subfamily G Member 1 (ABCG1) and/or Scavenger receptor class B type 1 (SR-BI) binding regions of the full-length apolipoprotein, thereby allowing binding to a myeloid cell. In particular embodiments, the apolipoprotein fragment comprises at least the alpha helices of the full-length apolipoprotein. These helices are hydrophilic on one side (interact with aqueous environment) and hydrophobic (interacts with lipids in the particle) on the other side.

In particular embodiments, the fragment may be a N- and/or C-terminally truncated form of the full-length peptide, polypeptide or protein from which it is derived.

In particular embodiments, the fragment may comprise at least about 30%, e.g., at least about 50% or at least about 70%, preferably at least about 80%, e.g., at least about 85%, more preferably at least about 90%, and yet more preferably at least about 95% or even about 99% of the amino acid sequence length of said full-length peptide, polypeptide, or protein from which it is derived. For example, insofar not exceeding the length of the full-length peptide, polypeptide, or protein, the fragment may include a sequence of ≥5 consecutive amino acids, or ≥10 consecutive amino acids, or ≥20 consecutive amino acids, or ≥30 consecutive amino acids, e.g., ≥40 consecutive amino acids, such as for example ≥50 consecutive amino acids, e.g., ≥60, ≥70, ≥80, ≥90, ≥100, or ≥200, consecutive amino acids of the corresponding full-length peptide, polypeptide, or protein.

In particular embodiments, the apolipoprotein component, such as an apolipoprotein fragment, comprises the myeloid-binding portion of full-length apolipoprotein.

In particular embodiments, the apolipoprotein component is an apolipoprotein mutant comprising a mutation that allows chemical conjugation of the apolipoprotein to a targeting body, wherein the targeting body is a molecule capable of binding a molecule on the cell surface of a target cell. In particular embodiments, the apolipoprotein component may also be an apolipoprotein mutant comprising a serine to cysteine substitution, such as the mutant as defined by SEQ ID NO: 9, 10, 11 or 12 as described elsewhere herein.

Peptide sequences for the different proteins described herein, or nucleic acid sequences for the genes encoding the different proteins described herein, are readily available to the skilled person, for example from the UCSC Genome Browser (http://genome.ucsc.edu/), Ensembl genome browser (https://www.ensembl.org) and NCBI (https://www.ncbi.nlm.nih.gov/protein). Consensus sequences for different proteins or genes are readily derived from these sources, although it is understood a certain variation may be present due to, but not limited to, genetic variation and multiple splice variants of the gene. Therefore, when referring to a specific protein, this should be interpreted to encompass sequence variations due to genetic variation and splice variants. Therefore, when used herein when referring to a certain protein, this should be interpreted as the corresponding consensus protein sequence as retrieved from the Ensembl genome browser, or the consensus nucleic acid (gene) sequence as retrieved from the Ensembl genome browser, or a protein sequence 80%, 85%, 90%, 91%, 92%, 93%, 94%, 95%, 96%, 97%, 98%, 99% or 100% identical to the corresponding consensus protein sequence as retrieved from the Ensembl genome browser, or a gene sequence 70%, 75%, 80%, 85%, 90%, 91%, 92%, 93%, 94%, 95%, 96%, 97%, 98%, 99% or 100% identical to the corresponding consensus gene sequence as retrieved from the Ensembl genome browser, or a nucleic acid sequence encoding a protein 80%, 85%, 90%, 91%, 92%, 93%, 94%, 95%, 96%, 97%, 98%, 99% or 100% identical to the corresponding consensus protein sequence as retrieved from the Ensembl genome browser.

apolipoprotein mimetics are synthetic peptides or proteins that mimic the function or structure of apolipoproteins. Several apolipoprotein mimetics are known and for example Wolska et al. (Cells. 2021 March; 10 (3): 597., incorporated by reference in its entirety) review different apoA1, apoE and apoC-Il mimetics described in the literature. For example, aapoA1 mimetic peptides have largely been designed based on their ability to efflux cholesterol from cells. As this process has not been shown to depend upon a specific protein-protein interaction, most apoA 1 mimetic peptides are simply just amphipathic helices and, in fact, many have no primary amino acid homology to apoA1.

Exemplary aapoA 1 mimetics are aapoA1 mimetic 18A, aapoA1 mimetic 2F and aapoA1 mimetic 37 pA.

For example, apoE has several putative atheroprotective functions, many different types of apoE-based peptides have been reported. One of the main goals in the design of these peptides is to facilitate the hepatic clearance of apoB-containing lipoproteins. As apoE can only bind to its receptor when bound to lipids, these peptides usually have not only the receptor-binding motif from the N-terminal domain of apoE, but also a lipid-binding region based on the C-terminal domain of apoE or some other sequence.

For example apoC-II mimetics have been described either based on a shortened first helix (18A) linked to the LPL-activation domain of apoC-II, or mimetics where both the first and second helix are based on the native apoC-Il helices with amino acid substitutions to enhance bihelical binding to lipoproteins.

Therefore, when used herein, an apolipoprotein mimetic refers to a synthetic protein or peptide which shares a structural and/or functional feature with the respective apolipoprotein. For example, the shared structural feature may be a primary, secondary or tertiary peptide structure such as the peptide sequence, presence of structures such as an alpha helix or beta sheet or three-dimensional structure of the peptide, or the functional feature may be a similarity in binding to a certain target such as a receptor. Preferably the apolipoprotein is capable of binding lipids, more preferably forming lipid particles, in a similar manner as the corresponding apolipoprotein.

In particular embodiments, the apolipoprotein mimetic may be able to bind to a myeloid cell to the same or a similar extent as the respective apolipoprotein. For example, the apolipoprotein mimetic of apoA1 is preferably able to bind to a myeloid cell to the same or a similar extent as apoA1.

In an embodiment the modified apolipoprotein is a combination of apoA1 with a targeting body. In an embodiment the modified apolipoprotein is a combination apoA 1 mimetic with a targeting body. In an embodiment the modified apolipoprotein is a combination apoE with a targeting body.

In an embodiment the modified apolipoprotein is a combination of an apolipoprotein component (e.g. an apoA1 mutant, preferably a human apoA1 mutant), with a targeting body. In an embodiment the modified apolipoprotein is a combination of an apoA1 mimetic with a targeting body. In an embodiment the modified apolipoprotein is a combination apoE with a targeting body.

By means of an example, the human apoA 1 protein sequence is annotated under NCBI Genbank (http://www.ncbi.nlm.nih.gov/) accession number NP_001304947.1 (isoform 1 preproprotein), and Uniprot (www.uniprot.org) accession number P02647.1.

In particular embodiments, the apolipoprotein component is apoA1 (e.g. as derived from the human precursor of apoA1 as defined by SEQ ID NO. 9, of which the first 18 amino acids form the signal peptide) or an apolipoprotein component derived from apoA1 (e.g. as defined by SEQ ID NO. 10, 11 or 12). In particular embodiments, the apolipoprotein component is human apoA1 as defined by SEQ ID NO. 13.

For example, in order to chemically conjugate the apolipoprotein component to a targeting body, a reactive handle may be used. Therefore, apolipoprotein components derived from apoA1 comprising a cysteine in the place of a serine at position 147 (e.g. as defined by SEQ ID NO. 11) or 279 (e.g. as defined by SEQ ID NO: 12) could be useful to prepare a modified apolipoprotein. In an embodiment, the apolipoprotein component comprises, consists essentially of or consists of an amino acid sequence at least 90%, at least 91%, at least 92%, at least 93%, at least 94%, at least 95%, at least 96%, at least 97%, at least 98%, at least 99% or 100% identical to SEQ ID NO. 9, SEQ ID NO. 14, SEQ ID NO. 15, SEQ ID NO. 10, SEQ ID NO. 11 or SEQ ID NO. 12, or an amino acid sequence encoded by a nucleic acid with a sequence at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to SEQ ID NO. 17, SEQ ID NO. 18, SEQ ID NO. 19, SEQ ID NO. 20, SEQ ID NO. 21 or SEQ ID NO. 22.

It is noted that the sequence as defined by SEQ ID NO. 14, SEQ ID NO. 10, SEQ ID NO. 11 or SEQ ID NO. 12 comprise N-terminally the amino acid sequence GLVPRGSIDD (SEQ ID NO. 16), which is a thrombin cleavage site. For example, the sequence as defined by SEQ ID NO. 15 comprises N-terminally a 6His tag followed by the amino acid sequence GLVPRGSIDD (SEQ ID NO. 16). Here, the thrombin cleavage site as could be used to remove the N-terminal His tag from the peptide.

In an embodiment, the apolipoprotein component comprises, consists essentially of or consists of, an amino acid sequence at least 90%, at least 91%, at least 92%, at least 93%, at least 94%, at least 95%, at least 96%, at least 97%, at least 98%, at least 99% or 100% identical to SEQ ID NO. 10, wherein SEQ ID NO. 10 comprises a cysteine at position 7 of SEQ ID NO. 10.

In an embodiment, the apolipoprotein component comprises, consists essentially of or consists of, an amino acid sequence at least 90%, at least 91%, at least 92%, at least 93%, at least 94%, at least 95%, at least 96%, at least 97%, at least 98%, at least 99% or 100% identical to SEQ ID NO. 11, wherein SEQ ID NO. 10 comprises a cysteine at position 150 of SEQ ID NO. 11.

In an embodiment, the apolipoprotein component comprises, consists essentially of or consists of, an amino acid sequence at least 90%, at least 91%, at least 92%, at least 93%, at least 94%, at least 95%, at least 96%, at least 97%, at least 98%, at least 99% or 100% identical to SEQ ID NO. 12, wherein SEQ ID NO. 10 comprises a cysteine at position 239 of SEQ ID NO. 12.

When used herein, when referring to a modified apolipoprotein the term fusion protein should be interpreted as an apolipoprotein and covalently attached thereto a targeting body. The covalent attachment may be due to the in frame coding of a peptide or protein sequence by the nucleotide sequence that encodes the fusion protein. Alternatively, the covalent attachment may be due to covalent linkage of the targeting body to the apolipoprotein, for example via a sulfur bond such as a thioether bond, formed at a cysteine residue of the apolipoprotein. Further the payload, may if it is a peptide or protein, optionally be a fusion protein with the modified apolipoprotein. It is understood that the targeting body and/or the payload and/or the apolipoprotein (or mimetic or derivative thereof) may include the site-specific incorporation of non-natural amino acids such as para-azidophenylalanine, which can be used in subsequent (strain-promoted) “click” (conjugation) reactions with alkyne modified reagents.

The modified apolipoprotein may comprise a linker, such as a flexible linker, between the apolipoprotein, apolipoprotein derivative, apolipoprotein mimetic or apolipoprotein mimetic derivative and the targeting body. The linker may be a glycine-serine linker, such as a (GGS) n-linker, wherein n is 0, 1, 2, 3, 4, 5, 6, 7, 8, 9 or 10, preferably a (GGS) 4 (SEQ ID. 23)-linker.

In particular embodiments, the modified apolipoprotein may comprise one or more tags, such as at the N- and/or C-terminal end of the modified apolipoprotein. The one or more tags, such as a 6His-tag or strep-tag may allow purification of the modified apolipoprotein.

The targeting body may be covalently attached to any portion of the apolipoprotein, apolipoprotein derivative, apolipoprotein mimetic or apolipoprotein mimetic derivative. A linker, such as a flexible linker, may be used to allow such covalent attachment.

In particular embodiments, the targeting body is located N- or C-terminally of said apolipoprotein, apolipoprotein derivative, apolipoprotein mimetic or apolipoprotein mimetic derivative in said modified apolipoprotein.

When used herein an immune response refers to a reaction which occurs within an organism by the immune system. The immune response may be the innate immune response or the adaptive immune response or the complement immune system. Immune responses when referred herein include but are not limited to: secretion of a pro-inflammatory molecule; secretion of an anti-inflammatory molecule; phagocytosis; antibody production, presentation or secretion; antigen presentation; activation, proliferation, suppression or differentiation of an immune cell; binding of an immune cell to a target, or initiation of an immune related cellular signaling cascade.

### Payload

In an embodiment the modified apolipoprotein further comprises a payload. The payload may be bound to the modified apolipoprotein, for example by a covalent bond. When used herein a payload refers to a molecule which is to be delivered a target site. Non limiting examples are a nucleic acid or a nucleic acid analog, a therapeutic, a biologic, a cytokine, a chemokine, a hormone, a growth factor, or combinations thereof, although the skilled person may be aware of additional types of payloads. The payload may be an immunomodulatory molecule and interfere with, change, stimulate or suppress the innate immune response or the adaptive immune response or the complement immune system. The payload may be a protein, peptide or organic compound. The payload may be isolated or derived from a natural source, cloned or synthesized.

Therefore, in an embodiment the payload is selected from a nucleic acid or a nucleic acid analog, a therapeutic, a biologic, a cytokine, a chemokine, a hormone, a growth factor, or combinations thereof.

In an embodiment the payload may be a cytokine. Cytokines are known to the skilled person to be small proteins of approximately 5 to 20 kDa and are important in cell signaling. For example, a cytokine may refer to: a four-alpha-helix bundle family cytokine, such as the IL-2 subfamily, the interferon (IFN) subfamily or the IL-10 subfamily; the IL-1 family; the cysteine knot cytokines such as the TGFbeta family; the IL-17 family. Therefore, the cytokine is preferably selected from IL18, IL18BP, IL1A, IL1B, IL1F10, IL1F3/IL1RA, IL1F5, IL1F6, IL1F7, IL1F8, IL1RL2, IL1F9, IL33, BAFF, 4-1BBL, TNFSF8, CD40LG, CD70, CD95L/CD178, EDA-A1, TNFSF14, LTA/TNFB, LTB, TNFalpha, TNFSF10, TNFSF11, TNFSF12, TNFSF13, TNFSF15, TNFSF4, IFNA1, IFNA10, IFNA13, IFNA14, IFNA2, IFNA4, IFNA7, IFNB1, IFNE, IFNG, IFNZ, IFNA8, IFNA5/IFNaG, IFNw/IFNW1, CLCF1, CNTF, IL11, IL31, IL6, Leptin, LIF, OSM, IL10, IL19, IL20, IL22, IL24, IL28B, IL28A, IL29, TGF-beta 1/TGFB1, TGF-beta 2/TGFB2, TGF-beta 3/TGFB3. In a preferred embodiment the cytokine is selected from the IL-2 subfamily, the interferon subfamily, the IL-10 subfamily, the IL-1 family, the TGFbeta family, or the IL-17 family, or combinations thereof, more preferably wherein the cytokine is selected from IL-1B, IL-2, IL-4, IL-38, or combinations thereof.

In an embodiment the modified apolipoprotein is a fusion protein of a modified apolipoprotein with IL-1B. IL-1B is also known as IL1B, IL-1B IL1F2 or interleukin 1 beta, and is a cytokine protein that in humans is encoded by the IL1B gene. In an embodiment the modified apolipoprotein is a fusion protein of a modified apolipoprotein with IL-2. IL-2 is also known as IL2 TCGF, lymphokine or interleukin 2, and is an interleukin that regulates the activities of leukocytes that are responsible for immunity. In an embodiment the modified apolipoprotein is a fusion protein of a modified apolipoprotein with IL-4. IL-4 is also known as BSF-1, IL4 or interleukin 4, is a cytokine that induces differentiation of naive helper T cells. In an embodiment the modified apolipoprotein is a fusion protein of a modified apolipoprotein with IL-38. IL-38 is also known as IL38, IL1F10 interleukin 38, interleukin 1 family member 10 or IL1-theta, and is a protein that in humans is encoded by the IL1F10 gene.

In an embodiment the payload may be a chemokine. The chemokine is preferably selected from CCL1/TCA3, CCL11, CCL12/MCP-5, CCL13/MCP-4, CCL14, CCL15, CCL16, CCL17/TARC, CCL18, CCL19, CCL2/MCP-1, CCL20, CCL21, CCL22/MDC, CCL23, CCL24, CCL25, CCL26, CCL27, CCL28, CCL3, CCL3L3, CCL4, CCL4L1/LAG-1, CCL5, CCL6, CCL7, CCL8, CCL9, CX3CL1, CXCL1, CXCL10, CXCL11, CXCL12, CXCL13, CXCL14, CXCL15, CXCL16, CXCL17, CXCL2/MIP-2, CXCL3, CXCL4, CXCL5, CXCL6, CXCL7/Ppbp, CXCL9, IL8/CXCL8, XCL1, XCL2, FAM19A1, FAM19A2, FAM19A3, FAM19A4 and FAM19A5. In an alternative embodiment the chemokine is selected from a CC chemokine, a CXC chemokine, a C chemokine, a CX3C chemokine or combinations thereof.

In an embodiment the payload is a hormone. Hormones are known to the skilled person to be signaling molecules in multicellular organisms, that are transported to distant organs to regulate physiology and behavior. In an embodiment the hormone is selected from Adrenaline (also known as epinephrine), Melatonin, Noradrenaline (also known as norepinephrine), Triiodothyronine, Thyroxine, Dopamine, Prostaglandins, Leukotrienes, Prostacyclin, Thromboxane, Amylin (also known as Islet Amyloid Polypeptide), Anti-Müllerian hormone (also known as Müllerian-inhibiting factor/hormone), Adiponectin, Adrenocorticotropic hormone (also known as corticotropin), Angiotensinogen, Angiotensin, Antidiuretic hormone (also known as vasopressin, arginine vasopressin), Atrial natriuretic peptide (also known as atriopeptin), Brain natriuretic peptide, Calcitonin, Cholecystokinin, Corticotropin-releasing hormone, Cortistatin, Enkephalin, Endothelin, Erythropoietin, Follicle-stimulating hormone, Galanin, Gastric inhibitory polypeptide, Gastrin, Ghrelin, Glucagon, Glucagon-like peptide-1, Gonadotropin-releasing hormone, Growth hormone-releasing hormone, Hepcidin, Human chorionic gonadotropin, Human placental lactogen, Growth hormone, Inhibin, Insulin, Insulin-like growth factor (also known as somatomedin), Leptin, Lipotropin, Luteinizing hormone, Melanocyte stimulating hormone, Motilin, Orexin, Osteocalcin, Oxytocin (also known as pitocin), Pancreatic polypeptide, Parathyroid hormone, Pituitary adenylate cyclase-activating peptide, Prolactin (also known as leuteotropic hormone), Prolactin-releasing hormone, Relaxin, Renin, Secretin, Somatostatin (also known as growth hormone-inhibiting hormone or growth hormone release-inhibiting hormone or somatotropin release-inhibiting factor or somatotropin release-inhibiting hormone), Thrombopoietin, Thyroid-stimulating hormone (also known as thyrotropin), Thyrotropin-releasing hormone, Vasoactive intestinal peptide, Guanylin or Uroguanylin.

In an embodiment the payload is a growth factor. Growth factors are known to the skilled person as naturally occurring substances capable of stimulating cell proliferation, wound healing, and occasionally cellular differentiation. In an embodiment the growth factor is selected from Adrenomedullin (AM), Angiopoietin (Ang), Autocrine motility factor, Bone morphogenetic proteins (BMPs), Ciliary neurotrophic factor (CNTF), Leukemia inhibitory factor (LIF), Interleukin-6 (IL-6), Macrophage colony-stimulating factor (M-CSF), Granulocyte colony-stimulating factor (G-CSF), Granulocyte macrophage colony-stimulating factor (GM-CSF), Epidermal growth factor (EGF), Ephrin A1, Ephrin A2, Ephrin A3, Ephrin A4, Ephrin A5, Ephrin B1, Ephrin B2, Ephrin B3, Erythropoietin (EPO), Fibroblast growth factor (FGF), Fibroblast growth factor 1 (FGF1), Fibroblast growth factor 2 (FGF2), Fibroblast growth factor 3 (FGF3), Fibroblast growth factor 4 (FGF4), Fibroblast growth factor 5 (FGF5), Fibroblast growth factor 6 (FGF6), Fibroblast growth factor 7 (FGF7), Fibroblast growth factor 8 (FGF8), Fibroblast growth factor 9 (FGF9), Fibroblast growth factor 10 (FGF10), Fibroblast growth factor 11 (FGF11), Fibroblast growth factor 12 (FGF12), Fibroblast growth factor 13 (FGF13), Fibroblast growth factor 14 (FGF14), Fibroblast growth factor 15 (FGF15), Fibroblast growth factor 16 (FGF 16), Fibroblast growth factor 17 (FGF17), Fibroblast growth factor 18 (FGF18), Fibroblast growth factor 19 (FGF19), Fibroblast growth factor 20 (FGF20), Fibroblast growth factor 21 (FGF21), Fibroblast growth factor 22 (FGF22), Fibroblast growth factor 23 (FGF23), Foetal Bovine Somatotrophin (FBS), Glial cell line-derived neurotrophic factor (GDNF), Neurturin, Persephin, Artemin, Growth differentiation factor-9 (GDF9), Hepatocyte growth factor (HGF), Hepatoma-derived growth factor (HDGF), Insulin, Insulin-like growth factor-1 (IGF-1), Insulin-like growth factor-2 (IGF-2), IL-1, IL-2, IL-3, IL-4, IL-5, IL-6, IL-7, Keratinocyte growth factor (KGF), Migration-stimulating factor (MSF), Macrophage-stimulating protein (MSP), also known as hepatocyte growth factor-like protein (HGFLP), Myostatin (GDF-8), Neuregulin 1 (NRG1), Neuregulin 2 (NRG2), Neuregulin 3 (NRG3), Neuregulin 4 (NRG4), Brain-derived neurotrophic factor (BDNF), Nerve growth factor (NGF), Neurotrophin-3 (NT-3), Neurotrophin-4 (NT-4), Placental growth factor (PGF), Platelet-derived growth factor (PDGF), Renalase (RNLS)-Anti-apoptotic survival factor, T-cell growth factor (TCGF), Thrombopoietin (TPO), Transforming growth factors, Transforming growth factor alpha (TGF-α), Transforming growth factor beta (TGF-β), Tumor necrosis factor-alpha (TNF-a), Vascular endothelial growth factor (VEGF), WNT1, WNT2, WNT2B, WNT3, WNT3A, WNT4, WNT5A, WNT5B, WNT6, WNT7A, WNT7B, WNT8A, WNT8B, WNT9A, WNT9B, WNT10A, WNT10B, WNT11 and WNT16. In a preferred embodiment the growth factor is selected from VEGF, EGF, CNTF, LIF, Ephrins, FGF, GDNF, HDF, HDGF, IGF, KGF, MSF, NRG, BDNF, NGF, Neurotrophin, PGF, PDGF, RNLS, TCGF, TGF, TNF, WNT or combinations thereof.

In an embodiment the payload is a hematopoietic growth factor. In an embodiment, the hematopoietic growth factor is selected from IL-3, CSF-1 (M-CSF), GM-CSF, G-CSF, a member of the IL-12 family of interleukins or erythropoietin or combinations thereof.

### Lipid Nanoparticles

It is envisioned the modified apolipoproteins as described herein may be used as proteins or as lipid nanoparticles. As described above apolipoprotein may circulate as such (meaning not incorporated in a lipoprotein or lipid nanoparticle). This application may be suitable to deliver payload, for example a cytokine, to a target site. It is known that apolipoproteins can circulate as proteins but may also form lipoproteins in situ. It may however also be advantageous to include the modified apolipoprotein in a lipid nanoparticle. Therefore, in an aspect the invention relates to a lipid nanoparticle comprising one or more modified apolipoproteins as described as herein. Without being bound to any hypothesis, it is believed that the apolipoprotein, apolipoprotein derivative, apolipoprotein mimetic or apolipoprotein mimetic derivative may function as a scaffold to help the formation of the nanoparticle together with the phospholipids, and sterols.

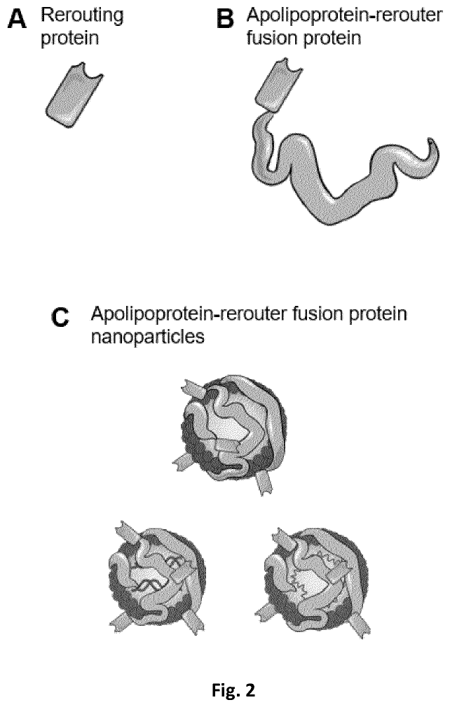

The apolipoprotein, apolipoprotein derivative, apolipoprotein mimetic or apolipoprotein mimetic derivative forms part of the lipid nanoparticle structure. In particular embodiments, at least a part of said modified apolipoprotein is exposed to the environment (i.e. aqueous environment) surrounding said lipid nanoparticle. Typically, part of the apolipoprotein, apolipoprotein derivative, apolipoprotein mimetic or apolipoprotein mimetic derivative is exposed to the environment surrounding said lipid nanoparticle (e.g. see FIG. 2). Furthermore, fusion of a targeting body to the apolipoprotein, apolipoprotein derivative, apolipoprotein mimetic or apolipoprotein mimetic derivative typically allows said targeting body to be wholly exposed to the environment surrounding said apolipoprotein lipid nanoparticle (e.g. see FIG. 2). In other words, in particular embodiments, the targeting body is not embedded within the lipid nanoparticle. As a result thereof, said targeting body may move freely and exert its natural function(s), such as its cell targeting function.

In particular embodiments, the lipid nanoparticle is not a phospholipid bilayer.

In particular embodiments, the phospholipid is selected from a phosphatidylcholine (PC), a phosphatidylethanolamine (PE), a phosphatidylserine and a phosphatidylglycerol or combinations thereof. In further particular embodiments, the phospholipid is selected from the group consisting of 1,2-diphytanoyl-sn-glycero-3-phosphocholine (PHPC), dilauroylphosphatidylcholine (DLPC), dimyristoylphosphatidylcholine (DMPC), dipalmitoylphosphatidylcholine (DPPC), distearoylphosphatidylcholine (DSPC), dioleoylphosphatidylcholine (DOPC), dilauroylphosphatidylglycerol (DLPG), dimyristoylphosphatidylglycerol (DMPG), dipalmitoylphosphatidylglycerol (DPPG), distearoylphosphatidylglycerol (DSPG), dioleoylphosphatidylglycerol (DOPG), dilauroyl phosphatidylethanolamine (DLPE), dimyristoyl phosphatidylethanolamine (DMPE), dipalmitoyl phosphatidylethanolamine (DPPE), distearoyl phosphatidylethanolamine (DSPE), dilauroyl phosphatidylserine (DLPS), dimyristoyl phosphatidylserine (DMPS), dipalmitoyl phosphatidylserine (DPPS), distearoyl phosphatidylserine (DSPS), 1-palmitoyl-2-oleoyl-sn-glycero-3-phosphocholine (POPC), 1,2-dioleoyl-sn-glycero-3-phosphoethanolamine (DOPE), or combinations thereof.

In particular embodiments, the sterol is selected from cholesterol, desmosterol, stigmasterol, β-sitosterol, ergosterol, hopanoids, hydroxysteroid, phytosterol, steroids, hydrogenated cholesterol, campesterol, zoosterol, or combinations thereof. The modified apolipoprotein may further be bound to a payload. The apolipoprotein may also be an apolipoprotein mimetic or derivative. Typically two types of nanoparticles can be distinguished: disc-like nanoparticles and spherical nanoparticles. Both comprise an outer layer of phospholipid, sterol and the modified apolipoprotein as described herein. The two differ in that the spherical nanoparticles further comprise a substantially hydrophobic core, while the disc-like nanoparticle lack a core and consequently form a disc-like shape. The core may typically comprise lipids, but not necessarily so. Preferably, the lipid nanoparticle is a sphere. Spherical nanoparticles enable linking of (many) more fusion proteins depending on the size than, for example, disc-like shape nanoparticles. Furthermore, spherical nanoparticles facilitate the encapsulation of larger payloads, such as mRNA or gene editing components, or a larger amount of payload (e.g. in case of small molecule drugs or small lipophilic payloads), compared to disc-like nanoparticles.

In particular embodiments, wherein the lipid nanoparticle comprises a payload, the lipid nanoparticle comprises a core surrounded by a surface layer, wherein the core comprises the payload and the surface layer comprises the apolipoprotein, apolipoprotein derivative, apolipoprotein mimetic or apolipoprotein mimetic derivative, the phospholipids, the targeting body, and the sterols.

In an embodiment, the invention relates to a lipid nanoparticle comprising an outer layer and optionally a core, wherein the outer layer comprises:
a phospholipid;
a sterol; and
the modified apolipoprotein as defined in any one of the preceding claims; and wherein the core, if present, comprises at least one core component selected from:
   a lipid, a cationic lipid, or a polyvalent molecule. When referring to an outer layer of a lipid nanoparticle the outer layer is essentially a phospholipid monolayer comprising additional components, at least cholesterol and modified apolipoprotein.

When used herein a lipid nanoparticle refers to an assembly of phospholipids and sterol and one or more apolipoproteins that is soluble in an aqueous solution. The particles may comprise lipids, in which case the lipids are encapsulated by the phospholipids and sterols. The nanoparticles may comprise further components such as additional proteins or a payload. Therefore, in an embodiment the lipid nanoparticle as defined herein further comprises lipids.

In particular embodiments, the lipid nanoparticle comprises a native (e.g. unfused) apolipoprotein, apolipoprotein derivative, apolipoprotein mimetic or apolipoprotein mimetic derivative, in addition to the apolipoprotein, apolipoprotein derivative, apolipoprotein mimetic or apolipoprotein mimetic derivative which forms part of the modified apolipoprotein as described herein. In a further embodiment, the lipid nanoparticle as defined herein further comprises a payload as defined herein above. The payload may for example be a pharmaceutical compound. The lipid nanoparticle core is particularly suitable for lipophilic payloads but may also be used for amphipathic molecules. The pharmaceutical compound may be an organic compound, peptide, protein, nucleic acid or nucleic acid analog, biologic or lipid. Therefore, in an embodiment the lipid nanoparticle further comprises a payload, preferably wherein the payload is selected form a nucleic acid or a nucleic acid analog, a therapeutic, a biologic or combinations thereof. Alternatively the payload may be bound to the outer layer of the lipid nanoparticle, e.g. to the (modified) apolipoprotein, or the payload may be comprised in the phospholipid layer.

For example, the payload may be a nucleic acid or a nucleic acid analog. Examples may be but are not limited to mRNA, siRNA, sgRNA, miRNA, piRNA, snRNA, snoRNA, srRNA or tsRNA. The nucleic acid analogue may be peptide nucleic acid (PNA), Morpholino and locked nucleic acid (LNA), as well as glycol nucleic acid (GNA), threose nucleic acid (TNA) and hexitol nucleic acids (HNA), or mixtures or combinations thereof. It is understood that if a nucleic acid payload is included in the lipophilic core of the nanoparticle, the charged backbone of the nucleic acid needs to be neutralized, for example by a cationic lipid.

In certain embodiments, the lipid nanoparticle comprises a nucleic acid and a cationic or ionizable cationic lipid.

In certain embodiments, the nucleic acid and the cationic or ionizable cationic lipid are comprised by the core, and the modified apolipoprotein and the phospholipid are comprised by the outer layer.

The present invention thus also encompasses a lipid nanoparticle comprising an outer layer and a core, wherein the outer layer comprises:
a phospholipid;
a sterol; and
the modified apolipoprotein as taught herein; and
   wherein the core comprises a nucleic acid and a cationic or ionizable cationic lipid.

Nucleic acid containing apolipoprotein particles of this kind are described in WO/2022/268913, the entire contents of which are incorporate by reference herein. The nanoparticles are engineered to complex nucleic acids, which are hydrophilic in nature, using helper molecules to draw the nucleic acids into the hydrophobic nanoparticle core. To this end, cationic hydrophobic molecules are employed. The cationic group can complex with the anionic phosphate groups in the sugar phosphate backbone via ionic interactions. The hydrophobic part of the helper molecule forms a shell around the hydrophilic nucleic acid molecule. The cationic helper molecules can be either permanently charged or ionizable. They comprise a wide variety of molecules, commercially available or synthesized in house, but they need to adhere to two general criteria: 1) A positively charged group to enable complexation with the negatively charged sugar phosphate backbone. 2) A hydrophobic part to form a hydrophobic shell and enable integration in the nanoparticle core. The content of cationic material in nanoparticle formulations may range from a cationic-to-anionic ratio of 1:1 to 25:1. This ratio, often referred to as the N/P (nitrogen/phosphate) ratio, is based on the number of positive charges in the (ionizable) cationic lipid (often nitrogen-based) versus the number of negative charges in the nucleic acid payload (usually phosphate). Accordingly, the N/P ratio is the ratio between the cumulative molar amount of cationic and/or ionizable groups in the cationic or ionizable lipid component(s) (N) and the cumulative molar amount of phosphate groups in the nucleic acid component(s) (P). In particular embodiments, the N/P ratio of the nanoparticles as taught herein is from 1 to 25, from 1 to 20, from 1 to 15, from 1 to 12, from 1 to 9, from 1 to 6, or from 1 to 3. For example, the N/P ratio of the nanoparticles as taught herein may be 3, 6, 9 or 12.

Without being bound to theory, present inventors believe that the nucleic acid-containing nanoparticles described herein have an outer layer comprising mainly the modified apolipoprotein, phospholipid and optional sterol, and a core comprising cationic or ionizable cationic lipid and the nucleic acid. More particularly, the core of the nanoparticle comprises an assembly of nucleic acid interacting with the (ionizable) cationic lipid, wherein this core of the nanoparticle is surrounded by a lipid shell comprising, consisting essentially of or consisting of the modified apolipoprotein, phospholipid and optional sterol. According to this understanding, the nucleic acid is located within (i.e. on the inside) of the nanoparticle, and is not located at the outer surface of the nanoparticle and/or is not exposed to the surroundings of the nanoparticle. In particular embodiments, the payload (i.e. nucleic acid) of the nanoparticles of this invention is not bound by ionic interactions at the outside (surface) of the particle. Binding of nucleic acid to the outside surface of the particle is undesired as the nucleic is left exposed to the immediate surroundings, presumably making the particles more toxic as well as leading to fast (bio)-degradation of the nucleic acid payload. According to this understanding, the modified apolipoprotein is located at the outer surface of the nanoparticle and/or is exposed to the surroundings of the nanoparticle.

The core of the nanoparticle may be solid and not have or bear a significant aqueous void or reservoir in the core. In particular embodiments, the core of the nanoparticle is non-aqueous.

In certain embodiments, the core of the nanoparticle is not surrounded by a lipid bi-layer, such as present in vesicle-like or liposomal particles with lipid bi-layers surrounding an aqueous core.

In particular embodiments, the nanoparticles do not comprise synthetic (non-natural) hydrophilic polymers or (lipid) conjugates of such polymers, such as most notably polyethylene-glycol (PEG). As a result thereof, such nanoparticles do not elicit unwanted immune responses, especially upon repeated administration.

In certain embodiments, the nanoparticle core further comprises a filler, preferably a filler selected from a triacylglyceride and a cholesterol acyl ester, or combinations thereof, such as wherein the triacylglyceride is tricaprylin and/or wherein the cholesterol acyl ester is cholesteryl caprylate and/or cholesteryl oleate. Cholesteryl acetate may also be employed as filler material. Yet other filler materials that can be applied are di-glycerides or tri-glycerides or other esters derived from C1-C18 carboxylic acids, preferably C6-C18 fatty acids, where these carboxylic acids and fatty acids may be saturated or unsaturated. Preferably, the filler is a tri-glyceride derived from C6-C18 fatty acids are preferred. Besides nucleic acids and cationic helper molecules, additional hydrophobic filler molecules can be included in the core of nanoparticle formulations. Their main application is to alter nanoparticle physicochemical properties and/or improve stability.

In certain embodiments, the nucleic acid is RNA, DNA or a nucleic acid analogue.

In certain embodiments, the RNA is microRNA (miRNA), small interfering RNA (SiRNA), piwi-interacting RNA (piRNA), small nuclear RNA (snRNA), small nucleolar RNA (snoRNA), transfer RNA (tRNA), RNA-derived small RNA (tsRNA), small regulatory RNA (srRNA), messenger RNA (mRNA), modified mRNA, ribosomal RNA (rRNA), long non-coding RNA (lncRNA), or guide RNA (gRNA), or combinations thereof and/or modifications thereof.

In certain embodiments, the DNA is single stranded or double stranded DNA.

In certain embodiments, the nucleic acid is an antisense oligonucleotide and the antisense oligonucleotide is single strand DNA or RNA consisting of nucleotide or nucleoside analogues containing modifications of the phosphodiester backbone or the 2′ ribose.

In certain embodiments, the nucleotide or nucleoside analogues are selected from locked nucleic acid (LNA), bridged nucleic acid (BNA), morpholino or peptide nucleic acid (PNA), glycol nucleic acid (GNA), threose nucleic acid (TNA), hexitol nucleic acids (HNA), or mixtures or combinations thereof.

In certain embodiments, the nucleic acid is conjugated, and the nucleic acid conjugate is incorporated into the nanoparticle of the invention. Nucleic acid conjugates include lipid conjugates with for example phospholipids or with sterols such as cholesterol or with hydrophobic alkyl chains. Nucleic acid conjugates also include conjugates with oligomers or polymers. Preferably, these oligomers or polymers are of a hydrophobic nature.

In certain embodiments, the nucleic acid is incorporated as such or ‘as is’ within the nanoparticle, meaning that the nucleic acid is not being conjugated.

When used herein, the term ionizable cationic lipid refers to a lipid which has a neutral charge at physiological pH (e.g. at pH 7 to 7.5, preferably at pH 7.3 to 7.5, such as at-pH 7.4) and which is protonated or positively charged at a lower pH (e.g. at pH 1 to 5, preferably at pH 1 to 4, such as at pH 4). It is understood that ionizable cationic lipids are particularly useful, as they may be protonated at low pH thus facilitating binding to the hydrophilic nucleic acid. By subsequently raising the pH the lipids may become (partly) neutral further facilitating inclusion in a hydrophobic environment, e.g. the hydrophobic core of a nanoparticle. Alternatively, and without being bound to theory, the ionizable lipids may remain positively charged within the nanoparticles, even though the pH of the surrounding aqueous solution has been raised to physiological pH, such as about 7.4, due to the action of the surface layer of the nanoparticle that comprises phospholipid, optionally sterol, and modified apolipoprotein, and/or due to the non-aqueous environment within the nanoparticle. Furthermore, ionizable cationic lipids are theorized to facilitate the endosomal escape of the nucleic acid in the target cells, where due to the low pH the ionizable cationic lipid will be protonated.

Non-limiting examples of ionizable cationic lipids are DLin-DMA (2-[2,2-bis (octadeca-9, 12-dienyl)-1, 3-dioxolan-4-yl]-N, N-dimethylethanamine), DLin-KC2-DMA (2-[2,2-bis[(9Z, 12Z)-octadeca-9, 12-dienyl]-1,3-dioxolan-4-yl]-N, N-dimethylethanamine)
and DLin-MC3-DMA ([(6Z,9Z,28Z,31Z)-heptatriaconta-6,9,28,31-tetraen-19-yl] 4-(dimethylamino) butanoate) as represented by formula 1 below:

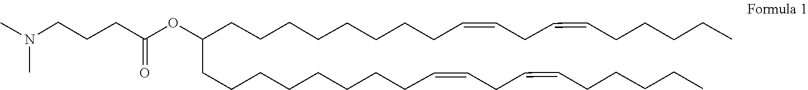

Indeed, a broad range of ionizable cationic lipids (including lipidoids) can be employed for preparing the nanoparticles of this invention, as various series of ionizable cationic lipids have been developed and reported on in literature. Further non-limiting examples include molecules cKK-E12, C12-200, L319, Acuitas-A9, Moderna-L5, TT3 and ssPalmE (such as described in, for example, Witzigmann et al., Advanced Drug Delivery Reviews 159 (2020) 344-363; doi.org/10.1016/j.addr.2020.06.026).

The ionizable lipid may further be an ionizable triglyceride. A non-limiting example is the compound represented by formula 2:

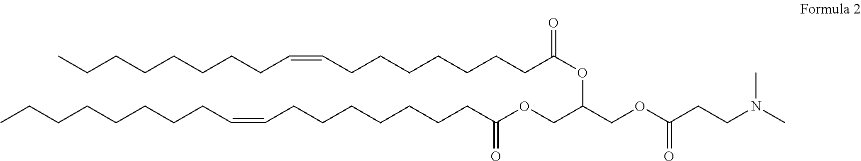 [SMILES: CCCCCCCC/C=C\CCCCCCCC(=O)OCC(COC(=O)CCN(C)C)OC(=O)CCCCCCC/C=C\CCCCCCCC]

The ionizable lipid may further be a cholesterol ester (also named a cholesteryl ester). A non-limiting example is represented by formula 3:

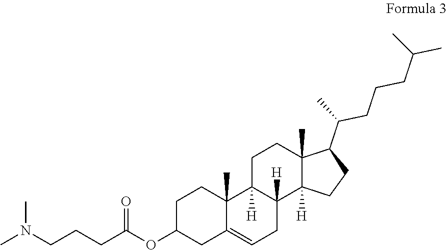

When used herein, the term cationic lipid refers to a positively charged lipid at physiological pH (e.g. pH 7.4). Non-limiting examples of cationic lipids are DOTMA (1,2-di-O-octadecenyl-3-trimethylammonium propane), DOGS (2,5-bis(3-aminopropylamino)-N-[2-[di (heptadecyl) amino]-2-oxoethyl] pentanamide), DOSPA (2-[3-[4-(3-aminopropylamino) butylamino]propylcarbamoylamino]ethyl-[2,3-bis [[(Z)-octadec-9-enoyl] oxy] propyl]-dimethylazanium) and DOTAP (1,2-dioleoyl-3-trimethylammonium-propane). Other examples include any ionizable cationic lipid molecules wherein the tertiary amine moiety has been converted to a quaternary ammonium moiety, for example by alkylation, such as by methylation (-Me), ethylation (-Et), benzylation (-Bn) or ethoxylation (—CH₂CH₂—OH). The resultant quaternary ammonium molecule has a permanent positive (cationic) charge, and accordingly also bears a counter anion, for example a chloride anion.

In an embodiment, only ionizable cationic lipids are used to prepare the nucleic acid-containing nanoparticles as taught herein. Accordingly, in an embodiment, the nanoparticles as taught herein do not comprise cationic lipids.

In an embodiment, only cationic lipids are used to prepare the nucleic acid-containing nanoparticles as taught herein. Accordingly, in an embodiment, the nanoparticles as taught herein do not comprise ionizable cationic lipids.

In an embodiment, a combination of ionizable cationic lipids and cationic lipids are used to prepare the nucleic acid-containing nanoparticles as taught herein.

When referring to the nucleic acid-containing nanoparticles as taught herein, the term “payload” in particular refers to the nucleic acid, preferably in combination with the cationic and/or ionizable cationic lipids.

The term “lipid” is well known in the art, and as used herein may in particular be considered to encompass both lipids, i.e. naturally occurring hydrophobic biomolecules such as for example fatty acids, mono-, di- or tri-glycerides of fatty acids, sterol (derivatives) or phospholipids, and lipid-like biomolecules. It is noted that the cationic lipids or ionizable cationic lipids (or lipidoids) described herein are typically not lipids within the most narrow interpretation of the term, i.e. naturally occurring hydrophobic biomolecules such as for example fatty acids, mono-, di- or tri-glycerides of fatty acids, sterol (derivatives) or phospholipids, but are lipid-like biomolecules that resemble lipid biomolecules, i.e. they preferably contain groups that are biocompatible (such as e.g. esters or amides), and/or are constructed using naturally occurring building blocks (e.g. fatty acids, glycerol, cholesterol).

In certain embodiments, the cationic or ionizable cationic lipid is selected from an ionizable cationic ester of a long chain alcohol, an ionizable cationic ester of a diglyceride or an ionizable cationic ester of a sterol or combinations thereof.

The ionizable cationic ester of a long chain alcohol may for example be an ester of a tertiary amine with a carboxy group such as a compound with the formula (CH₃)₂N (CH₂)ₙCOOH, wherein n is an integer of 1 or more, for instance n is 1 to 12; for example 3-dimethylamino-propionic acid or 4-dimethylamino-butyric acid or 5 dimethylamino-pentanoic acid. The ester is formed with a long chain alcohol. The long chain alcohol is preferably a primary or secondary alcohol with a straight or branched chain length of 8 or more carbon atoms, for example 8, 9, 10, 11, 12, 13, 14, 15, 16, 17, 18, 19, 20 or more.

The ionizable cationic ester of a diglyceride is preferably a diacyl glycerol (i.e. a di-glyceride) coupled at the 1 or 2 position with a tertiary amine with a carboxy group such as a compound with the formula (CH₃) 2N (CH₂),COOH, wherein n is an integer of 1 or more, for instance n is 1 to 12; for example 3-dimethylamino-propionic acid or 4-dimethylamino-butyric acid or 5-dimethylamino-pentanoic acid. The diacyl glycerol may comprise medium chain or long chain saturated or unsaturated fatty acids or derivatives or modifications thereof.

The ionizable cationic ester of a sterol is preferably an ester of sterol coupled at the hydroxyl group to a tertiary amine with a carboxy group such as a compound with the formula (CH₃)₂N(CH₂)ₙCOOH, wherein n is an integer of 1 or more, for instance n is 1 to 12; for example 3-dimethylamino-propionic acid or 4-dimethylamino-butyric acid or 5 dimethylamino-pentanoic acid. The sterol may be cholesterol, stigmasterol or β-sitosterol.

In the above, a carboxy compound is presented with the formula (CH₃) 2N (CH₂),COOH, wherein n is an integer of 1 or more. Instead of this compound, an alternative compound can be employed with the formula NH₂—(C═NH)—NH—(CH₂)ₙCOOH, wherein n is an integer of 1 or more, for instance n is 1 to 12. This carboxy compound comprises a guanidine group instead of a tertiary amine group.

The ionizable cationic lipid can for example be selected from the molecules as according to Formulas (I) to (V).

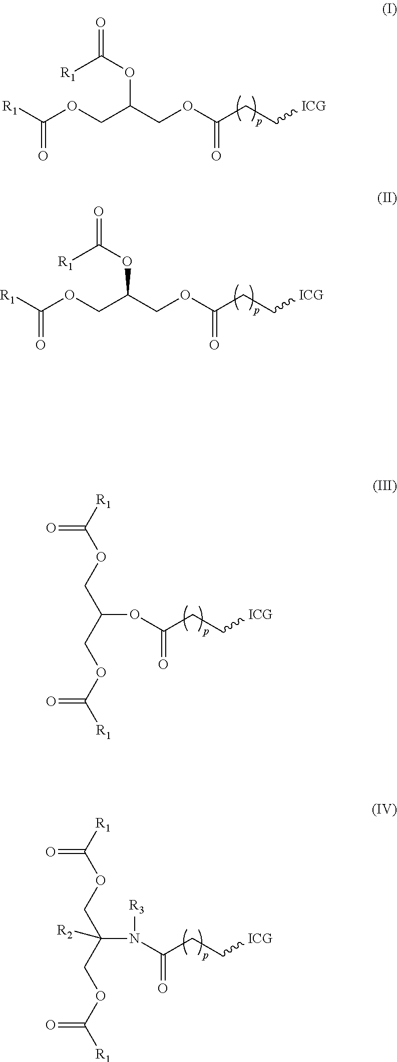

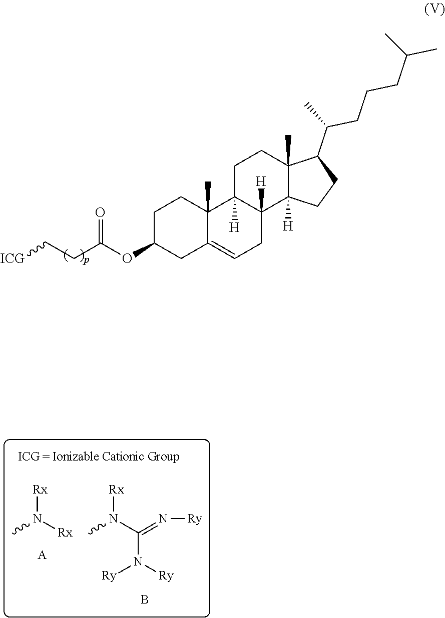

Formula (I) represents a tri-glyceride, wherein the ionizable cationic group (ICG) is comprised in the 1-position.

Formula (II) represents the same type of tri-glyceride as represented in Formula (I), albeit that the molecule is stereo-specifically defined in the naturally occurring configuration, i.e. as it would in a phospholipid: the ICG group is in the same position as the phosphate group is in a phospholipid.

Formula (III) represents a tri-glyceride, wherein the ionizable cationic group (ICG) is comprised in the 2-position.

Formula (IV) represents a di-ester (or a tri-ester), wherein the ionizable cationic group (ICG) is connected via the amide functionality.

Formula (V) represents a cholesteryl ester, wherein the ionizable cationic group (ICG) is connected via the ester functionality.

The ionizable cationic group (ICG) is connected via the wavy bond to the rest of the molecule for any of the Formulas (I) to (V), where the ICG can either represent a tertiary amine (ICG type A, or ICG-A) or it can represent a guanidine (ICG type B, or ICG-B).

In Formulas (I) to (IV), R1 can be independently selected for every position, and it represents a linear or branched C1-C19 alkyl, a linear or branched C1-C19 alkenyl, aryl, arylene-alkyl or alkylene-aryl group, wherein said alkyl or alkenyl group, optionally containing 5 heteroatoms, independently selected from O and N. Preferably, every R1-group within a specific molecule as according to any of the Formulas (I) to (IV) is the same R₁ group. Preferably, the R₁ group is a linear or branched C5-C19 alkyl group, or a linear or branched C5-C19 alkenyl group. When R₁ is an alkenyl group, this group preferably has one single double bond only. More preferably, the R₁ group is a linear or branched C9-C17 alkyl group or a linear or branched C5-C17 alkenyl group. Preferably R₁ is a linear C5-C15 alkyl group or a linear C17-C19 alkenyl. Carboxylic acids derived from R₁, i.e. R₁—COOH, are preferably naturally occurring fatty acid molecules such as capric acid, lauric acid, myristic acid, palmitic acid, stearic acid, palmiteic acid, oleic acid or linoleic acid. Preferred are the C10-C16 saturated fatty acids as well as oleic acid (C18, unsaturated).

The integer p is a discrete number and not an average value; p can be 0 to 11. Preferably, p is 0, 1, 2, 3, 4, 5, 6, 7, 8 or 9. More preferably, p is 1, 2, 3 or 4.

The R₂ group in Formula (IV) can be selected from a hydrogen, a methyl, an ethyl and a —CH₂—O—C(O)—R₁ₐ group (wherein R₁ₐ has the same meaning as R₁ defined above). Preferably, R₂ is a hydrogen, a methyl or a —CH₂—O—C(O)—R₁ group. More preferably, R₂ is a methyl.

The R₃ group in Formula (IV) can be selected from a hydrogen, aryl, arylene-alkyl, alkylene-aryl or a linear C₁-C₆ alkyl group. Preferably, R₃ is a hydrogen or a methyl. More preferably, R₃ is a hydrogen.

The Rₓ group in ICG-A can be independently selected for every position, and is selected from a methyl, an ethyl, a propyl and an ethylene-hydroxy (—CH₂—CH₂—OH) group, preferably it is a methyl group. Preferably, both Rₓ groups in ICG-A are the same groups, and they preferably are methyl groups.

The R_{y} group in ICG-B can be independently selected for every position from a hydrogen, a linear or branched C1-C18 alkyl, aryl, arylene-alkyl or alkylene-aryl group, wherein said alkyl group optionally contains up to 5 heteroatoms, independently selected from O and N. Preferably, the R_{y} group is selected from a hydrogen and a linear C₁-C₆ alkyl group. Even more preferably, the R_{y} group is a hydrogen. Preferably, all four R_{y}-groups in ICG-B are the same groups, and they preferably are hydrogens.

From Formulas (I) to (V), Formulas (I), (II) and (IV) are preferred. More preferred are Formulas (I) and (II).

From the ICGs ICG-A and ICG-B, ICG-A is preferred, i.e. tertiary amine ionizable cationic lipids are preferred.

The ionizable cationic lipid molecule as according to any one of the Formulas (I) to (V) has a molecular weight that is higher than 250 Dalton, preferably higher than 350 Dalton, more preferably higher than 450 Dalton. It has a molecular weight that is lower than 3000 Dalton, preferably lower than 1800 Dalton, more preferably lower than 1200 Dalton.

The molecules that are represented by Formulas (I) to (V) may exist in various isomeric forms such as rotamers, tautomers, stereoisomers or regiomers, and all of these are included in the scope of the present invention.

The ionizable cationic lipid as according to any one of the Formulas (I) to (V) preferably is a single compound, i.e. not a mixture of compounds. Accordingly, the purity of the ionizable cationic lipid of Formula (I) to (V) preferably is 50% or higher, preferably 80% or higher, more preferably 90% or higher, most preferably 95% or higher. In case the ionizable cationic lipid is a mixture of compounds, then this is preferably due only to the presence of undefined stereo-centers in the molecule. An example is the use of branched alkyl chains in the ionizable cationic lipid that are of racemic origin. Other examples are tri-glycerides wherein the substitution pattern over the three hydroxy-groups in the glycerol entity is not stereo-specifically defined.

Hence, in certain embodiments, the ionizable cationic lipid is a molecule according to any one of Formulae (I), (II), (III), (IV) or (V)

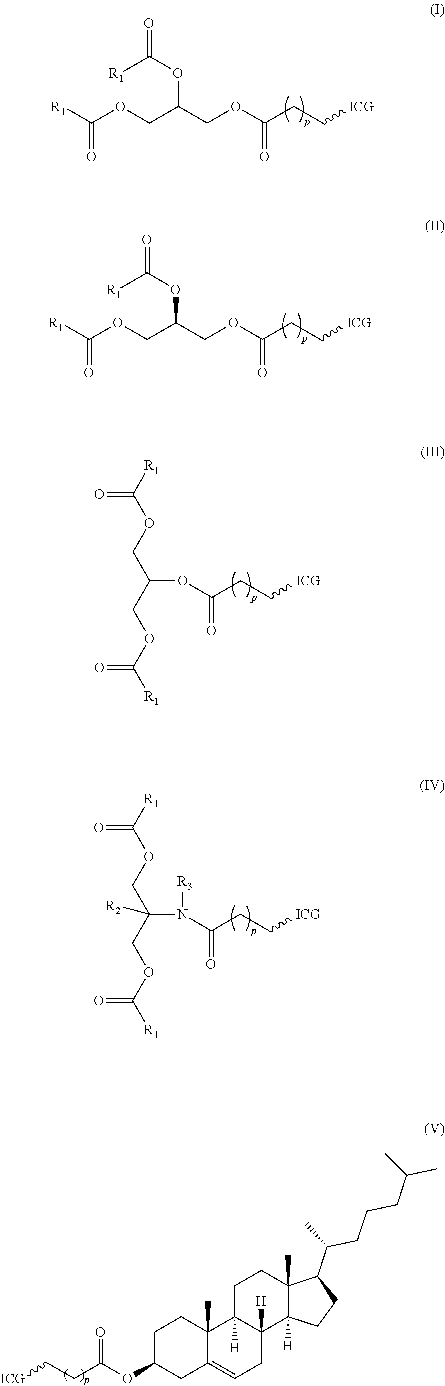

wherein ICG is

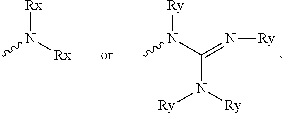

wherein the wavy line indicates the point of attachment to the compound of formulae (I), (II), (III), (IV) or (V);
p is an integer selected from 0, 1, 2, 3, 4, 5, 6, 7, 8, 9, 10 and 11;
each R¹ is independently selected from the group consisting of linear or branched C1-C19 alkyl, linear or branched C1-C19 alkenyl, aryl, arylene-alkyl and alkylene-aryl group, wherein said alkyl or alkenyl group optionally contains up to 5 heteroatoms, independently selected from O and N;
R₂ is selected from the group consisting of hydrogen, methyl, ethyl and a —CH₂—O—C(O)—R₁ₐ;
R₃ is selected from the group consisting of hydrogen, aryl, arylene-alkyl, alkylene-aryl and linear C₁-C₆ alkyl group;
R₁ₐ is selected from the group consisting of linear or branched C1-C19 alkyl, linear or branched C1-C19 alkenyl, aryl, arylene-alkyl and alkylene-aryl group, wherein said alkyl or alkenyl group optionally contains up to 5 heteroatoms, independently selected from O and N;
each R^{x} is independently selected from the group consisting of methyl, ethyl, propyl and —CH₂—CH₂—OH;
each R_{y} group is independently selected from the group consisting of hydrogen, linear or branched C1-C18 alkyl, aryl, arylene-alkyl or alkylene-aryl group, wherein said alkyl group optionally contains up to 5 heteroatoms, independently selected from O and N; or rotamers, tautomers stereoisomers or regioisomers thereof.

The term “alkyl” by itself or as part of another substituent refers to a hydrocarbyl group of formula CₙH₂ₙ₊₁ wherein n is a number greater than or equal to 1. Alkyl groups may be linear or branched and may be substituted as indicated herein. Generally, alkyl groups of this invention comprise from 1 to 18 carbon atoms, preferably from 1 to 17 carbon atoms, preferably from 1 to 15 carbon atoms, preferably from 1 to 6 carbon atoms, preferably from 1 to 5 carbon atoms, preferably from 1 to 4 carbon atoms, more preferably from 1 to 3 carbon atoms, still more preferably 1 to 2 carbon atoms. When a subscript is used herein following a carbon atom, the subscript refers to the number of carbon atoms that the named group may contain. For example, the term “C1-6alkyl”, as a group or part of a group, refers to a hydrocarbyl group of formula —CₙH₂ₙ₊₁ wherein n is a number ranging from 1 to 6. Thus, for example, “C1-6alkyl” includes all linear or branched alkyl groups with between 1 and 6 carbon atoms, and thus includes methyl, ethyl, n-propyl, i-propyl, butyl and its isomers (e.g. n-butyl, i-butyl and t-butyl); pentyl and its isomers, hexyl and its isomers. For example, “C1-5alkyl” includes all includes all linear or branched alkyl groups with between 1 and 5 carbon atoms, and thus includes methyl, ethyl, n-propyl, i-propyl, butyl and its isomers (e.g. n-butyl, i-butyl and t-butyl); pentyl and its isomers. For example, “C1-4alkyl” includes all linear or branched alkyl groups with between 1 and 4 carbon atoms, and thus includes methyl, ethyl, n-propyl, i-propyl, butyl and its isomers (e.g. n-butyl, i-butyl and t-butyl). For example “C1-3alkyl” includes all linear or branched alkyl groups with between 1 and 3 carbon atoms, and thus includes methyl, ethyl, n-propyl, i-propyl.

When the suffix “ene” is used in conjunction with an alkyl group, i.e. “alkylene”, this is intended to mean the alkyl group as defined herein having two single bonds as points of attachment to other groups. As used herein, the term “C1-6alkylene”, by itself or as part of another substituent, refers to C1-6alkyl groups that are divalent, i.e., with two single bonds for attachment to two other groups. Alkylene groups may be linear or branched and may be substituted as indicated herein. Non-limiting examples of alkylene groups include methylene (—CH₂—), ethylene (—CH₂—CH₂—), methylmethylene (—CH(CH₃)—), 1-methyl-ethylene (—CH(CH₃)—CH₂—), n-propylene (—CH₂—CH₂—CH₂—), 2-methylpropylene (—CH₂—CH(CH₃)—CH₂—), 3-methylpropylene (—CH₂—CH₂—CH(CH₃)—), n-butylene (—CH₂—CH₂—CH₂—CH₂—), 2-methylbutylene (—CH₂—CH(CH₃)—CH₂—CH₂—), 4-methylbutylene (—CH₂—CH₂—CH₂—CH(CH₃)—), pentylene and its chain isomers, hexylene and its chain isomers.

The term “alkenyl” as a group or part of a group, refers to an unsaturated hydrocarbyl group, which may be linear, or branched, comprising one or more carbon-carbon double bonds. When a subscript is used herein following a carbon atom, the subscript refers to the number of carbon atoms that the named group may contain. For example, the term “C2-6alkenyl” refers to an unsaturated hydrocarbyl group, which may be linear, or branched comprising one or more carbon-carbon double bonds and comprising from 2 to 6 carbon atoms. For example, C2-4alkenyl includes all linear, or branched alkenyl groups having 2 to 4 carbon atoms. Examples of C2-6alkenyl groups are ethenyl, 2-propenyl, 2-butenyl, 3-butenyl, 2-pentenyl and its isomers, 2-hexenyl and its isomers, 2,4-pentadienyl, and the like.

The term “aryl”, as a group or part of a group, refers to a polyunsaturated, aromatic hydrocarbyl group having a single ring (i.e. phenyl) or multiple aromatic rings fused together (e.g. naphthyl), or linked covalently, typically containing 6 to 24 carbon atoms, preferably 6 to 12 atoms; preferably 6 to 10, wherein at least one ring is aromatic. Examples of suitable aryl include C6-10aryl, more preferably C6-8aryl. Non-limiting examples of C6-12aryl comprise phenyl; biphenylyl; biphenylenyl; or 1- or 2-naphthanelyl; 1-, 2-, 3—, 4-, 5- or 6-tetralinyl (also known as “1,2,3,4-tetrahydronaphthalene); 1-, 2-, 3—, 4-, 5-, 6-, 7- or 8-azulenyl, 4-, 5-, 6 or 7-indenyl, 4- or 5-indanyl, 5-, 6-, 7- or 8-tetrahydronaphthyl; 1,2,3,4-tetrahydronaphthyl; and 1,4-dihydronaphthyl; 1-, 2-, 3—, 4- or 5-pyrenyl. When the suffix “ene” is used in conjunction with an aryl group; i.e. arylene, this is intended to mean the aryl group as defined herein having two single bonds as points of attachment to other groups. Suitable “C6-12arylene” groups include 1,4-phenylene, 1,2-phenylene, 1,3-phenylene, biphenylylene, naphthylene, indenylene, 1-, 2-, 5- or 6-tetralinylene, and the like. Where at least one carbon atom in an aryl group is replaced with a heteroatom, the resultant ring is referred to herein as a heteroaryl ring. The heteroatom may be selected from the group consisting of O, N, P and S; preferably O or N.

The term “alkylene-aryl”, as a group or part of a group, means a alkylene as defined herein, wherein at least one hydrogen atom is replaced by at least one aryl as defined herein. Alkylene-aryl groups typically contain 7 to 25 carbon atoms. Non-limiting examples of alkylene-aryl group include benzyl, phenethyl, dibenzylmethyl, methylphenylmethyl, 3-(2-naphthyl)-butyl, and the like. The term “arylene-alkyl”, as a group or part of a group, means a arylene as defined herein, wherein at least one hydrogen atom is replaced by at least one alkyl group as defined herein. Arylene-alkyl groups typically contain 7 to 25 carbon atoms.

Ester, amide, carboxylic acid and alcohol groups are defined hereunder, where Rp represents a hydrogen atom or a cyclic, linear or branched alkyl or alkylene groups. In groups that contain more than one Rp element, then these elements can be independently selected. An ester (functional) group or moiety as indicated in this document is to be understood as a group according to the formula:—C(O)—O—. An amide (functional) group or moiety as indicated in this document is to be understood as a group according to the formula:—NRp-C(O)—. A carboxylic acid (functional) group or moiety as indicated in this document is to be understood as a moiety or group according to the formula:—C(O) OH. An alcohol (or hydroxy) functional group or moiety as indicated in this document is to be understood as a group according to the formula:—OH.

The ionizable cationic lipids as according to any one of the Formulas (I) to (V) can be prepared by synthetic methods that are known in the art, such as without limitation as described in WO/2022/268913, such as in particular in the Examples section and especially Example 9 of WO/2022/268913.

The (ionizable) cationic lipid preferably can be processed from solutions. Accordingly, the (ionizable) cationic lipid is preferably soluble in solvents ranging in polarity. Therefore, the (ionizable) cationic lipid is preferably soluble in tricaprylin, in ethanol or in iso-propanol, more preferably in all three of these solvents. The solubility can be checked by stirring about 20 mg of the (ionizable) cationic lipid in about 1 gram of tricaprylin, ethanol or iso-propanol, and assessing whether all material spontaneously dissolves to create a clear/transparent solution with a concentration of about 2 w/w %. The test can be done at about 20° C. (room temperature) or at about 37° C. Preferably, the (ionizable) cationic lipid is soluble at room temperature.

The (ionizable) cationic lipid is preferably non-toxic, or it may have a limited and low toxicity, either on its own, or when bound to or tested together with nucleic acids, or assayed in the nanoparticle as taught herein. Toxicity cell tests can be executed by methods that are known in the art, such as for example by cell viability MTT assays, or by similar or comparable tests.

In certain embodiments,
the amount of the modified apolipoprotein, in particular the cumulative amount in case of two or more modified apolipoproteins, ranges from 0.08 to 2.0 mol %, such as from 0.10 to 2.0 mol %; and/or
the amount of phospholipid ranges from 5 to 90 mol %, such as from 15 to 90 mol %; and/or the amount of sterol ranges from 2.5 to 65 mol %, such as from 2.5 to 50 mol %; and/or
the amount of cationic or ionizable cationic lipid ranges from 5.0 to 80 mol %, such as from 8.0 to 80 mol %,
wherein the molar percentage is based solely on the combined amounts of the modified apolipoprotein, phospholipids, sterols and cationic or ionizable cationic lipids in the lipid nanoparticle.

Where a nanoparticle includes two or more distinct apolipoprotein components, such as an apolipoprotein and a modified apolipoprotein, or two or more modified apolipoproteins and optionally one or more apolipoprotein, etc., the cumulative amount of these apolipoprotein components is taken into account when quantitative relationships to other constituents of the nanoparticle are discussed.

In certain embodiments of nucleic acid-containing nanoparticles as taught herein, the amount of modified apolipoprotein (in case of two or more modified apolipoproteins, cumulatively) ranges from 0.05 to 2.0 mol %, such as from 0.10 to 2.0 mol % or from 0.08 to 0.5 mol %; and/or the amount of phospholipid ranges from 5 to 90 mol %, such as from 15 to 90 mol % or from 8.0 to 50 mol %; and/or the amount of sterol ranges from 2.5 to 65 mol %, such as from 2.5 to 50 mol % or from 4 to 65 mol %; and/or the amount of cationic or ionizable cationic lipid ranges from 5.0 to 80 mol %, such as from 8.0 to 80 mol % or from 5 to 65 mol % wherein the molar percentage is based solely on the combined amounts of the modified apolipoprotein, phospholipids, sterols and cationic and/or ionizable cationic lipids in the nanoparticle. These ranges contribute positively to the stability of the nanoparticles and their ability to incorporate nucleic acids.

In certain embodiments of nucleic acid-containing nanoparticles as taught herein, the amount of modified apolipoprotein (in case of two or more modified apolipoproteins, cumulatively) ranges from 0.01 to 2.0 mol %, such as from 0.05 to 1.0 mol %, or from 0.05 to 0.5 mol %, or from 0.05 to 0.4 mol %, or from 0.05 to 0.3 mol %, or from 0.05 to 0.2 mol %, or from 0.05 to 0.1 mol %, such as about 0.06, about 0.07, about 0.08, or about 0.09 mol %; and/or the amount of phospholipid ranges from 2 to 90 mol %, such as from 2 to 80 mol %, or from 2 to 70 mol %, or from 2 to 60 mol %, or from 2 to 50 mol %, or from 2 to 40 mol %, or from 2 to 30 mol %, or from 2 to 20 mol %, such as from 5 to 10 mol %, such as about 6, about 7, about 8, or about 9 mol %; and/or the amount of sterol ranges from 2.5 to 65 mol %, such as from 2.5 to 50 mol %, or from 5 to 50 mol %, or from 5 to 40 mol %, or from 5 to 30 mol %, or from 10 to 30 mol %, such as from 15 to 25 mol %, such as about 20, or about 21, or about 22 mol %; and/or the amount of cationic or ionizable cationic lipid ranges from 5.0 to 80 mol %, such as from 10 to 80 mol %, or from 10 to 70 mol %, or from 10 to 60 mol %, or from 10 to 50 mol %, or from 10 to 40 mol %, or from 10 to 30 mol %, or from 15 to 25 mol %, such as about 18, or about 19 or about 21 mol %; and/or
the amount of lipids, such as preferably triglycerides, ranges from 0 to 95 mol %, or from 0 to 90 mol %, such as from 1 to 95 mol %, or from 1 to 90 mol %, such as from 10 to 90 mol %, or from 20 to 80 mol %, or from 30 to 70 mol %, or from 40 to 60 mol %, or from 45 to 55 mol %, such as about 50, or about 51, or 52 mol %; and/or
the amount of nucleic acid, such as preferably RNA, e.g., mRNA, ranges from 0.01 to 1.0 mol %, such as from 0.01 to 0.1 mol %, or from 0.01 to 0.05 mol %, such as about 0.02, about 0.03, or about 0.04 mol %;
wherein the molar percentage is based solely on the combined amounts of the modified apolipoprotein, phospholipids, sterols, cationic and/or ionizable cationic lipids, lipids, such as preferably triglycerides, and nucleic acid, such as preferably RNA, in the nanoparticle. These ranges contribute positively to the stability of the nanoparticles and their ability to incorporate nucleic acids.

In certain embodiments of nucleic-acid containing nanoparticles as taught herein,
the amount of the modified apolipoprotein (in case of two or more modified apolipoproteins, cumulatively) ranges from 0.1 to 90 weight %;
the amount of nucleic acid ranges from 0.01 to 90 weight %;
the amount of phospholipid ranges from 0.1 to 95 weight %;
the amount of sterol ranges from 0.1 to 95 weight %; and/or
the amount of cationic and/or ionizable cationic lipid ranges from 0.1 to 95 weight %,
   wherein these weight percentages are based on the combined amounts of the modified apolipoprotein, the nucleic acid, the phospholipid, the sterol and the cationic and/or ionizable cationic lipid.

In an embodiment of nucleic-acid containing nanoparticles as taught herein, the amount of the apolipoprotein (in case of two or more modified apolipoproteins, cumulatively) ranges from 0.2 to 50 weight %, such as from 3 to 20 weight % or from 4 to 20 weight %, more preferably from 0.5 to 30 weight %, more preferably from 1 to 20 weight %.

In an embodiment of nucleic-acid containing nanoparticles as taught herein, the amount of nucleic acid ranges from 0.02 to 30 weight %, more preferably from 0.05 to 20 weight %, more preferably from 0.1 to 15 weight %, such as from 0.5 to 5 weight %.

In an embodiment of nucleic-acid containing nanoparticles as taught herein, the amount of phospholipid ranges from 0.2 to 60 weight %, more preferably from 1 to 50 weight %, such as from 10 to 50 weight %, more preferably from 3 to 40 weight %, such as from 10 to 40 weight %.

In an embodiment of nucleic-acid containing nanoparticles as taught herein, the amount of sterol ranges from 0.2 to 90 weight %, more preferably from 0.5 to 70 weight %, such as from 2 to 65 weight %, more preferably from 1 to 50 weight %, such as from 2 to 45 weight %, from 10 to 45 weight % or from 10 to 20 weight %.

In an embodiment of nucleic-acid containing nanoparticles as taught herein, the amount of cationic and/or ionizable cationic lipid ranges from 0.2 to 90 weight %, more preferably from 0.5 to 80 weight %, more preferably from 1 to 70 weight %, such as from 5 to 60 weight %, from 8 to 60 weight %, from 9 to 60 weight %, from 10 to 60 weight %, from 15 to 25 weight %, or from 20 to 60 weight %.

In an embodiment of nucleic-acid containing nanoparticles as taught herein, the amount of optional filler or filler molecule ranges from 0 to 90 weight %, more preferably from 0 to 80 weight %, more preferably from 0 to 70 weight %, such as from 0 to 65 weight %.

In particular embodiments of nucleic-acid containing nanoparticles as taught herein, the amount of optional filler or filler molecule ranges from 20 to 80 weight %, more preferably from 30 to 70 weight %, even more preferably from 30 to 65 weight %, such as from 40 to 65 weight %, from 45 to 55 weight % or from 30 to 60 weight %.

These weight percentages as indicated above are based on the combined amounts of the modified apolipoprotein, the nucleic acid, the phospholipid, the sterol and the cationic and/or ionizable cationic lipid, and optionally the filler material, i.e. these five or six components add up to 100% of the weight of the nanoparticle in the context of these statements. These weight percentage ranges contribute positively to the stability of the nanoparticles and their ability to incorporate nucleic acids.

In particular embodiments, the nanoparticle as taught herein does not comprise a filler or filler molecule.

In certain embodiments, the ratio of the modified apolipoprotein (in case of two or more modified apolipoproteins, cumulatively) to phospholipid based on percentage molar weight is between 1:25 and 1:400, more preferably between 1:50 and 1:200, even more preferably between 1:75 and 1:150.

In certain embodiments, the ratio of the modified apolipoprotein (in case of two or more modified apolipoproteins, cumulatively) to phospholipid based on weight is from 2:1 to 1:10, more preferably from 1:1 to 1:5, even more preferably from 1:1.5 to 1:4. These ranges contribute positively to the stability of the nanoparticles.

Alternatively, the payload may be a small organic compound such as a small molecule drug. Generally, the small organic compound is synthesized. The therapeutic may for example be an anticancer therapy such as a chemotherapy. Alternatively, the payload may be a biologic. When used herein, the term biologic is used to indicate a biopharmaceutical, also known as a biologic (al) medical product, and can be any pharmaceutical drug product manufactured in, extracted from, or semi-synthesized from biological sources. Biologics can be composed of sugars, proteins, nucleic acids, or complex combinations of these substances, or may be living cells or tissues.

In particular embodiments, the lipid nanoparticle has an average size of 10 to 100 nm, such as from 30 to 100 nm.

In particular embodiments, the ratio of modified apolipoprotein (in case of two or more modified apolipoproteins, cumulatively) to phospholipid based on percentage molar weight is between 1:25 and 1:400, more preferably between 1:50 and 1:200, even more preferably between 1:75 and 1:150. In particular embodiments, the ratio of modified apolipoprotein (in case of two or more modified apolipoproteins, cumulatively) as taught herein to phospholipid based on percentage molar weight is between 1:25 and 1:400, more preferably between 1:50 and 1:200, even more preferably between 1:75 and 1:150.

In particular embodiments, the ratio of modified apolipoprotein (in case of two or more modified apolipoproteins, cumulatively) to phospholipid based on weight is from 3:1 to 1:100. In particular embodiments, the ratio of modified apolipoprotein (in case of two or more modified apolipoproteins, cumulatively) as taught herein to phospholipid based on weight is from 3:1 to 1:100.

In certain embodiments, in lipid nanoparticles as taught herein, such as in particular but without limitation nanoparticles not containing a nucleic acid:
the amount of the modified apolipoprotein (in case of two or more modified apolipoproteins, cumulatively) ranges from 0.05 to 2.0 mol %, such as from 0.1 to 1.0 mol %, for example from 0.2 to 0.7 mol %, or from 0.2 to 0.5 mol %, such as preferably about 0.3 mol %; and/or
the amount of phospholipid ranges from 5 to 90 mol %, such as from 10 to 50 mol %, for example from 10 to 40 mol %, from 10 to 30 mol %, or from 10 to 20 mol %, such as about 11, about 12, about 13, about 14, about 15, about 16, about 17, about 18, or about 19 mol %; and/or
the amount of sterol ranges from 2.5 to 65 mol %, such as from 2.5 to 60 mol %, from 2.5 to 50 mol %, from 2.5 to 40 mol %, or from 2.5 to 30 mol %, or from 2.5 to 20 mol %, or from 2.5 to 10 mol %, such as from 2.5 to 5 mol %, such as about 3.0 or about 4.0, or about 5.0 mol %; and/or
the amount of lipids, such as preferably triglycerides, ranges from 1 to 95 mol %, such as from 10 to 95 mol %, or from 20 to 95 mol %, or from 30 to 95 mol %, or from 40 to 95 mol %, or from 50 to 95 mol %, or from 60 to 95 mol %, such as from 1 to 90 mol %, such as from 10 to 90 mol %, or from 20 to 90 mol %, or from 30 to 90 mol %, or from 40 to 90 mol %, or from 50 to 90 mol %, or from 60 to 90 mol %, such as from 60 to 80 mol %, or from 70 to 80 mol %, such as about 65, about 70, or about 75 mol %,
wherein the molar percentage is based solely on the combined amounts of the modified apolipoprotein, phospholipids, sterols, and lipids, such as preferably triglycerides in the lipid nanoparticle.

In a further aspect, the invention relates to a method of manufacturing a lipid nanoparticle as described herein, the method comprising the steps of:
a1) expressing and isolating a modified apolipoprotein as disclosed herein to obtain an isolated modified apolipoprotein; and/or
a2) chemically conjugating a targeting body to an apolipoprotein or apolipoprotein mimetic to obtain a modified apolipoprotein and isolating the modified apolipoprotein;
b) combining the isolated modified apolipoprotein obtained in step a1 and/or step a2 with phospholipids, sterols and optionally lipids to obtain a lipid nanoparticle.

In an embodiment step b) comprises:
b1) mixing, preferably rapid mixing, of lipid components in organic solvent with a nucleic acid in an aqueous buffer to produce nanoparticles, wherein the lipid components comprise a phospholipid, a sterol, a core component, and optionally a lipid; and wherein the aqueous buffer has a pH of 5.5 or lower, preferably 5.0 or lower; and b2) mixing, preferably rapid mixing, of lipid nanoparticles with the modified apolipoprotein to produce the nanoparticle at a pH between 5.5 and 9.0, preferably at a pH between 6.0 and 8.0, more preferably at a pH between 6.5 and 8.0.

It is understood that the modified apolipoprotein can be expressed as a chimeric fusion protein of the apolipoprotein with the targeting body or can be chemically conjugated to the targeting body or can be produced be a combination of these. Expression of chimeric proteins is known to the skilled person and can be used when the targeting body is a peptide or protein. It is well within the knowledge of the skilled person to use molecular techniques to produce a nucleic acid encoding such protein, for example by cloning targeting body encoding sequence in frame with an apolipoprotein (or mimetic or derivative) encoding sequence, for example at C or N terminal sequence encoding nucleotides. An advantage of using chimeric protein expression is that all expressed protein will be fusion protein.

Alternatively chemical conjugation may be used. Suitable methods for chemical conjugation of the targeting body to the apolipoprotein (or mimetic thereof) are known to the skilled person. Non limiting examples are strain promoted cycloaddition, aminolysis and Michael type addition. For example, an existing or introduced cysteine residue may be used on either the apolipoprotein or the targeting body. Introduction of a cysteine residue may be achieved by point mutation of a nucleotide in the encoding nucleotide sequence, or by introduction of a cysteine encoding codon. An advantage of chemical conjugation is that it is not limited to the use of peptide or protein sequences but can be applied to any type of organic molecule.

It is understood that the modified apolipoprotein, phospholipids, sterols and optional components may be rapidly mixed to obtain a lipid nanoparticle. Optionally added may be lipids and/or a payload as defined herein.

The present invention also encompasses a method for producing a lipid nanoparticle, comprising the step of:
a) rapid mixing of lipid components in organic solvent with a nucleic acid in an aqueous buffer to produce lipid nanoparticles, wherein the lipid components comprise a phospholipid, a sterol, a cationic lipid or ionizable cationic lipid, wherein the aqueous buffer has a pH of 5.0 or lower; and
b) rapid mixing of the lipid nanoparticles with one or more modified apolipoprotein as taught herein;
   to produce the lipid nanoparticle at a pH between 5.5 and 8.0, preferably between 6.0 and 8.0.

According to the above method, a two-step reaction is performed, where in the first step, a nucleic acid containing nanoparticle is formed, and in the next second step, modified apolipoprotein is included in the nanoparticle. Preferably, the first step is performed at low pH and the second step is performed at physiological pH.

The organic solvent may be an alcohol such as ethanol, iso-propanol, methanol, acetonitrile, dimethyl sulfoxide (DMSO), chloroform or combinations thereof. Preferred organic solvents are water mixable and non-toxic, for example ethanol and DMSO, or combinations thereof.

For example, the organic solvent may be from 96% to 100% of ethanol, preferably 100% ethanol.

Rapid mixing is known in the field and has for example been described in Hirota et al. BIOTECHNIQUES VOL. 27, NO. 2, p286-289; Jeffs et al., Pharm Res 22, 362-372 (2005); Kulkarni et al., ACS Nano 2018, 12, 5, 4787-4795

The aqueous buffer in step a) has a low pH to ensure that the ionizable cationic lipid is positively charged, allowing binding within and inclusion of the nucleic acid/cationic lipid complex in the particle. For example, the buffer may have a pH of 5.0 or lower, such as 4.9, 4.8, 4.7, 4.6, 4.5, 4.4, 4.3, 4.2, 4.1, 4.0, 3.9, 3.8, 3.7, 3.6, 3.5 or lower. The aqueous buffer may be any buffer that does not damage the nucleic acid. An exemplary buffer is sodium acetate at pH 4.0. The nanoparticle is then taken in an aqueous buffer with a pH of around 6 to 8, preferably 7 to 8 more preferably around 7.4. This may for example be achieved by dialysis with an aqueous buffer in the indicated pH range. A non-limiting example of an aqueous buffer suitable for this step is 155 mM PBS at pH 7.4, but it is understood that any buffer may be used that does not damage the nucleic acid.

In step b) the nanoparticle in an aqueous buffer at pH between 6 to 8, preferably between pH 7 to 8, is rapidly mixed with the modified apolipoprotein in an aqueous buffer at pH between 5.5 to 8, preferably 6 to 8, more preferably between pH 7 to 8, to obtain the nanoparticles.

The above described two-step formulation process as taught herein results in nucleic acid-containing nanoparticles with a broad set of desired and beneficial characteristics (stability, low toxicity or non-toxicity, high nucleic acid retention, nucleic acid activity, etc.). However, the described formulation method is non-limiting as other processes may also lead to nanoparticles with beneficial features.

A further aspect of the invention provides a pharmaceutical composition comprising the modified apolipoprotein as taught herein, the nucleic acid as taught herein or the lipid nanoparticle as taught herein, and a pharmaceutically acceptable carrier.

In a further aspect the invention relates to modified apolipoprotein as defined herein or the lipid nanoparticle as defined herein, or the lipid nanoparticle obtained or obtainable by the method as described herein, or the nucleic acid as defined herein, or the pharmaceutical composition as defined herein for use as a medicament. It is envisioned that either the modified apolipoprotein (e.g. by the action of an attached payload) or the payload comprised in the nanoparticle may be used to treat, ameliorate or alleviate a symptom in a subject. Therefore, in an embodiment modified apolipoprotein according to the first aspect or the lipid nanoparticle according to second aspect, or the lipid nanoparticle obtained or obtainable by the method of the third aspect of the invention or the nucleic acid as defined herein, or the pharmaceutical composition as defined herein for use in the treatment or prevention of an immune related disorder such as transplantation rejection, graft-versus-host disease (GVH), atherosclerosis, infection, inflammation, auto-immunity, allergy, cancer, a genetic disorder, a metabolic disorder, a neurological disorder or tissue trauma, preferably an immune related disorder such as transplantation rejection, atherosclerosis, infection, auto-immunity, cancer, a genetic disorder, a metabolic disorder, a neurological disorder or tissue trauma. Further embodiments relate to a method of treating an immune related disorder in a subject in need thereof, comprising administering to said subject a therapeutically effective amount of a modified apolipoprotein according to the first aspect or the lipid nanoparticle according to second aspect, or the lipid nanoparticle obtained or obtainable by the method of the third aspect of the invention or the nucleic acid as defined herein, or the pharmaceutical composition as defined herein to the subject in need thereof. Also provided herein is the use of a modified apolipoprotein according to the first aspect or the lipid nanoparticle according to second aspect, or the lipid nanoparticle obtained or obtainable by the method of the third aspect of the invention or the nucleic acid as defined herein, or the pharmaceutical composition as defined herein for the manufacture of a medicament for the treatment of an immune related disorder in a subject.

An immune related disorder as used herein comprises any disorder wherein the immune system plays a role in the disease development. An immune related disorder may refer to a disorder where the immune system is suppressed or where it is (over) activated. Examples of immune related disorders are cancer, infection, sepsis, autoimmune diseases, and cardiovascular diseases. Examples of autoimmune diseases are Type 1 diabetes, Rheumatoid arthritis (RA), Psoriasis/psoriatic arthritis, Multiple sclerosis (MS), Systemic lupus erythematosus (SLE), Inflammatory bowel disease (IBD), Addison's disease, Graves' disease, Sjögren's syndrome, Hashimoto's thyroiditis, Myasthenia gravis, Autoimmune vasculitis, Pernicious anemia, and Celiac disease. In an embodiment, the immune related disorder is selected from the group consisting g of transplantation rejection, graft-versus-host disease (GVH), atherosclerosis, infection, inflammation, auto-immunity, allergy, cancer, a genetic disorder, a metabolic disorder, a neurological disorder and tissue trauma, preferably transplantation rejection, atherosclerosis, infection, auto-immunity, cancer, a genetic disorder, a metabolic disorder, a neurological disorder or tissue trauma

In an embodiment the immune related disorder is selected from the group consisting of cancer, infection, sepsis, Type 1 diabetes, Rheumatoid arthritis (RA), Psoriasis/psoriatic arthritis, Multiple sclerosis (MS), Systemic lupus erythematosus (SLE), Inflammatory bowel disease (IBD), Addison's disease, Graves' disease, Sjögren's syndrome, Hashimoto's thyroiditis, Myasthenia gravis, Autoimmune vasculitis, Pernicious anemia, and Celiac disease.

In a further aspect, the invention relates to the use of a modified apolipoprotein as described herein or the lipid nanoparticle according as described herein, or the lipid nanoparticle obtained or obtainable by the method as described herein, or the nucleic acid as defined herein, or the pharmaceutical composition as defined herein in delivering a payload (e.g. a compound) to a target, preferably wherein the target is a cell, tissue, and/or organ, even more preferably wherein the target is a lymphoid cell, a myeloid cell, a tumor cell, or wherein the targeting body binds a bacterial, viral, fungal or parasitic protein or antigen, preferably wherein the lymphoid or myeloid cell is selected from: a monocyte, a macrophage, an M1-like macrophage, an M2-like macrophage, an eosinophil, a basophil, a mast cell, an NK cell, a B cell, a plasma cell, a regulatory T cell, a hematopoietic stem cell, a T helper cell such as Th1, Th2, Th17 or Th22, a dendritic cell, such as a plasmacytoid dendritic cell, a conventional DC 1 or a conventional DC 2, or a tumor-associated macrophage. In an embodiment the use is an ex vivo or in vitro use. In an alternative embodiment the use is an in vivo use. Typically, the apolipoprotein or targeting body will bind to a cell surface protein, such as a receptor. Therefore, the target may be a protein such as a receptor, a cell or cell type (expressing said protein), a tissue or tissue type (expressing said protein) or an organ (expressing said protein). It is understood that by choosing or adapting the targeting body the modified apolipoprotein can be targeted to different proteins. For example, the receptor binding domain of a ligand can be used to target a specific receptor. Alternatively known binding partners of cell surface proteins can be used to reroute the fusion protein (i.e. the modified apolipoprotein).

A further aspect of the invention provides an in vitro or ex vivo method for introducing a nucleic acid in a cell, the method comprising contacting the nucleic acid-containing lipid nanoparticle as taught herein, or the nucleic acid-containing lipid nanoparticle obtained or obtainable by the method as taught herein, with a cell.

A further aspect of the invention provides an in vivo method for introducing a nucleic acid in a cell, the method comprising contacting the nucleic acid-containing lipid nanoparticle as taught herein, or the nucleic acid-containing lipid nanoparticle obtained or obtainable by the method as taught herein, with a cell.

A further aspect of the invention provides the nucleic acid-containing lipid nanoparticle as taught herein, or the nucleic acid-containing lipid nanoparticle obtained or obtainable by the method as taught herein, for use in the in vivo delivery of a nucleic acid to a subject.

A further aspect of the invention provides a method for the in vivo delivery of a nucleic acid, the method comprising administering the nucleic acid-containing lipid nanoparticle as taught herein, or the nucleic acid-containing lipid nanoparticle obtained or obtainable by the method as taught herein, to a subject.

A further aspect of the invention provides a method for treating a disease or disorder in a subject in need thereof, the method comprising administering a therapeutically effective amount of the nucleic acid-containing lipid nanoparticle as taught herein, or the nucleic acid-containing lipid nanoparticle obtained or obtainable by the method as taught herein, to the subject.

The diseases may be any of the diseases as discussed herein, such as for example an immune related disorder such as transplantation rejection, graft-versus-host disease (GVH), atherosclerosis, infection, inflammation, auto-immunity, allergy, cancer, a genetic disorder, a metabolic disorder, a neurological disorder or tissue trauma, preferably transplantation rejection, atherosclerosis, infection, auto-immunity, cancer, a genetic disorder, a metabolic disorder, a neurological disorder or tissue trauma.

A purpose of the nucleic acid-containing nanoparticles described herein is to deliver a nucleic acid to a cell or to deliver a nucleic acid therapy to a subject. The nucleic acid may be for example an mRNA encoding a peptide or protein of interest which is to be expressed in the cell, or may comprise a short nucleic acid such as an siRNA, shRNA intended to interfere in gene expression (e.g. gene silencing), or it may comprise a component of the CRISPR-Cas or a related gene editing system (e.g. gRNA) to induce a mutation in the genome of the cell. Therefore in general the mode of action of the nucleic acid (the payload of the nanoparticle) is in the cytoplasm or the nucleus. Therefore the nanoparticle preferably has at least the following properties: 1) it allows targeting of the intended target cell, and 2) it allows delivery of the payload where it can assert its action (thus in most cases in the cytoplasm or nucleus of the target cell).

It is understood that the nucleic acid therapy comprising nanoparticles may be administered to a subject in need thereof. Depending on the target cells or tissue, the administration may be parenteral, e.g. intravenous, intramuscular or subcutaneous. The administration may further be oral, sublingual, topical, rectal, nasal (inhaled) or vaginal. Further the targeting of the target tissue or cells is determined by the proper choice of the modified apolipoprotein. In an embodiment, the use of the nanoparticle or composition comprises delivering a nucleic acid to the myeloid compartment or the spleen. This may for example be achieved by intravenous parenteral administration.

The nanoparticles of certain embodiments of the invention, after systemic injection, can target tissues (spleen, bone marrow) that are associated with the presence of immune cells.

In certain embodiments, the nucleic acid-containing nanoparticle as taught herein, or the composition as taught herein for use in immunotherapy.

In an aspect the invention relates to the nanoparticle, including the nucleic acid containing nanoparticles, as taught herein, or the composition comprising them for use in the treatment of a disease by stimulating or inhibiting an innate immune response, preferably wherein said disease is a disease that would benefit from stimulating or inhibiting the innate immune response in a subject, such as a disease characterized by a defective innate immune response, more preferably wherein said disease to be treated is a cancer, a cardiovascular disease, an autoimmune disorder or xenograft rejection.

Therefore the nanoparticles may be used in the treatment of any disease relating the immune system such as any immune disorder, or for the treatment of any disease or disorder where modulating the immune response is deemed a viable treatment option.

In a further aspect, the invention relates to a nucleic acid encoding a fusion protein (i.e. modified apolipoprotein) as defined herein. The nucleic acid may be comprised in a vector such as a viral vector for stable integration in a cell, or an expression vector to enable transient expression.

It will be appreciated by persons skilled in the art that numerous variations and/or modifications may be made to the above-described aspects and/or embodiments, without departing from the broad general scope of the present invention. The present aspects and/or embodiments are, therefore, to be considered in all respects as illustrative and not restrictive. The present invention includes the following non-limiting examples.

### EXAMPLES

### Example 1. Generation of Cell Marker Specific Apolipoprotein-Nanobody Fusion Proteins

aNPs are lipid-based nano-sized formulations (diameter ˜30-200 nm) with a hydrophobic core and apolipoproteins covering the outer surface. Apolipoproteins are helical proteins with inherent affinity for lipid layers due to their amphiphilic character. There are several classes of apolipoproteins, and all can be used as a structural component for aNP formulations. The presence of apolipoprotein modulates the biological behaviour of the aNP. For example, apolipoprotein A1 interacts with cells via scavenger receptor class B type 1 (SRB1) and ATP-binding cassette transporter ABCA1. This increases interactions of the aNP with myeloid cells in lymphoid organs. However, therapeutically functionalized aNPs have shown to have little effect on immune cell types without these mentioned receptors. Additionally, increasing targeting specificity within the myeloid compartment can also increase efficiency of the therapeutics. Re-routing aNPs to specific cell types would therefore increase the power of this modular platform.

Nanobodies are single antigen specific polypeptides consisting of the variable domain of the heavy chain (VH domain) of heavy chain only antibodies found in camelids and are also called VHH domains or single-domain antibodies (sdAbs). The nanobodies are low in complexity and highly stable and soluble, giving them greater therapeutic potential compared to the human antibody equivalent. They consist of a conserved framework of β-sheets, and three variable complementarity determining regions (CDRs). A nanobody's specificity arises from the variation in these CDR loops. Nanobodies lack the hydrophobic patch that is present in the framework of human antibodies to increase the interaction between the VH and VL domain. Nanobodies have four hallmark mutations in this region which are key for their stability and solubility. The conserved framework can be modified using point mutations to humanize the nanobodies to limit immunogenicity. Apolipoprotein-nanobody fusion proteins combine the particle forming ability of the apolipoprotein with the targeting ability of the nanobody. Fusion proteins are generated by molecular cloning of the genes for the apolipoprotein and the nanobody, which are fused with an appropriate amino acid linker to allow for sufficient space between the apolipoprotein and the nanobody. The fusion proteins will subsequently be recombinantly expressed in E. coli and purified using purification tags like a histidine tag. Including the fusion protein during aNP production then generates cell-type specific aNPs. The targeted cell type can easily be determined by changing the nanobody sequence to one specific for a desired cell marker.

Nanobodies specific for desired cell markers are engineered via a yeast surface display method and directed evolution. Machine-learning-guided generative modelling is used to introduce variation in the directed evolution process to create nanobody sequences with higher affinity for their target. This process is repeated until a sequence is selected with the desired binding affinity.

### Example 2. Re-Routed aNP Evaluation

In vitro evaluation of cell-specific aNPs is performed to analyze aNP-cell interactions. aNPs are incubated with human peripheral blood mononuclear cells (PBMCs) and flow cytometry is used to evaluate aNP binding to immune cells. After incorporation of a therapeutic compound, therapeutic efficacy of the aNP is assessed in vitro.

Subsequently, in vivo efficiency of cell targeting by re-routed aNPs is assessed in mice with a humanized immune system. Organ- and cell-specific biodistribution is investigated, as well as toxicology and therapeutic efficacy of the aNPs.

### Example 3. Implementation

Re-routed aNPs have the potential to specifically deliver therapeutic compounds to desired immune cell types, thereby modulating the immune response. A major advantage of the re-routed aNP platform technology is the possibility to easily exchange the nanobody and therapeutic payload. Re-routed aNPs can therefore be implemented as immunotherapies that promote the immune response to treat e.g., cancer or infectious diseases, or to dampen the immune response to treat e.g., autoimmune diseases or during organ transplantation.

### Example 4. Fusion Proteins of an Apolipoprotein Fused to a Rerouting Protein or Peptide (Targeting Body) and Incorporation Thereof into Lipid Nanoparticles

### Materials and Methods

Expression and purification of VHHCD8-apoA1 fusion protein: A small culture of ClearColi cells transformed with pET20b-VHHCD8-apoA1 plasmid and pDiscoTune plasmid was started in LB medium with 100 μg/mL ampicillin. The next day, 40 mL of small culture was diluted in 1 liter of 2YT medium to start large cultures and rhamnose was added at a final concentration of 50 UM to induce T7 lysozyme on the pDiscoTune plasmid. The culture was grown at 37° C. and 150 rpm until an OD600 of 0.6-0.8, then isopropyl β-d-1-thiogalactopyranoside (IPTG) was added at a final concentration of 0.1 mM to induce expression. The induced culture was incubated overnight at 18° C. and 150 rpm. Induced bacterial cultures were pelleted and cells were resuspended in lysis buffer (20 mM Tris, 500 mM NaCl, pH 7.9). Benzonase Nuclease (Merck Millipore) and one complete™ EDTA-free Protease Inhibitor Cocktail tablet (Roche) per 50 mL cell suspension was added and the cell suspension was incubated at 4° C. while stirring. The suspension was subsequently homogenized three times at 15000-20000 psi using the Avestin Emulsiflex C3. The cell lysate was kept on ice at all times. After lysis, cell lysate was centrifuged to pellet insoluble cell debris and supernatant was flown through an Immobilized Metal Chelate Affinity Chromatography (IMAC) column containing immobilized nickel ions. The column was washed with 8 column volumes of buffer A (20 mM Tris, 500 mM NaCl, 10 mM imidazole, pH 7.9), then 8 column volumes of buffer A50 (20 mM Tris, 500 mM NaCl, 50 mM imidazole, pH 7.9). To elute VHHCD8-apoA1, 8 column volumes of buffer A500 (20 mM Tris, 500 mM NaCl, 500 mM imidazole, pH 7.9) was applied to the column. All fractions of the purification steps were collected and analyzed with SDS-PAGE. The buffer of fractions containing purified VHHCD8-apoA1 was changed to PBS using Amicon Ultracentrifugal Filters (Amicon). To store VHHCD8-apoA1, aliquots were snap-frozen in liquid nitrogen and stored at −70° C.

The VHHCD8-apoA1 fusion protein has the sequence as defined by SEQ ID NO: 1, or is encoded by a sequence as defined by SEQ ID NO: 2, which comprises a linker between apoA1 and VHHCD8 comprising a cysteine.

SEQ ID NO: 1
MTGQVQLQESGGGLVQAGGSLRLSCAASGFTFDDYAIGWFRQAPGKEREG
VSCINNSDGSGYVADSVKGRFTISSDNAKNTVYLQMNSLKPEDTAVYYCA
AAGDGGCYYGLDYWGKGTQVTVSSGGLECKLSGSGSGSDDPPQSPWDRVK
DLATVYVDVLKDSGRDYVSQFEGSALGKQLNLKLLDNWDSVTSTFSKLRE
QLGPVTQEFWDNLEKETEGLRQEMSKDLEEVKAKVQPYLDDFQKKWQEEM
ELYRQKVEPLRAELQEGARQKLHELQEKLSPLGEEMRDRARAHVDALRTH
LAPYSDELRQRLAARLEALKENGGARLAEYHAKATEHLSTLSEKAKPALE
DLRQGLLPVLESFKVSFLSALEEYTKKLNTQGDHHHHHH
SEQ ID NO: 2
ATGACCGGTCAGGTTCAGTTACAGGAGTCCGGTGGGGGACTGGTGCAGGC
CGGGGGTTCTCTGCGCCTTAGCTGCGCTGCCTCCGGTTTCACCTTCGATG
ATTACGCTATTGGCTGGTTCCGTCAGGCTCCTGGCAAGGAGCGTGAAGGA
GTATCCTGTATCAACAATTCGGACGGGAGTGGGTATGTAGCGGACAGTGT
TAAAGGTCGCTTTACGATTTCCTCCGACAACGCCAAGAACACCGTCTACC
TTCAAATGAACTCCCTGAAGCCAGAAGATACCGCTGTTTATTACTGTGCC
GCAGCGGGTGACGGTGGTTGTTACTACGGCTTGGACTACTGGGGTAAAGG
AACCCAGGTTACTGTCTCATCCGGGGGACTGGAATGCAAGCTTTCAGGGT
CAGGTAGTGGATCTGATGATCCGCCGCAGAGTCCATGGGATCGCGTGAAG
GACCTGGCCACTGTGTACGTGGATGTGCTCAAAGACAGCGGCAGAGACTA
TGTGTCTCAGTTTGAAGGATCCGCCTTGGGCAAACAATTGAACCTTAAGC
TGCTGGACAACTGGGACAGCGTGACGTCCACCTTCAGCAAGCTGCGCGAA
CAGCTCGGCCCTGTGACCCAGGAATTCTGGGATAACCTGGAAAAGGAGAC
AGAGGGCCTGCGCCAGGAGATGAGCAAGGATCTGGAGGAGGTGAAGGCCA
AGGTGCAGCCGTACCTGGACGACTTCCAGAAGAAGTGGCAGGAGGAGATG
GAGCTCTACCGCCAGAAGGTGGAGCCGCTGCGCGCAGAGCTGCAGGAGGG
CGCGCGCCAGAAGCTGCACGAGCTGCAAGAGAAGCTGAGCCCACTGGGCG
AGGAGATGCGCGACCGCGCGCGCGCCCATGTCGACGCGCTGCGCACGCAT
CTGGCGCCGTACAGCGACGAGCTGCGCCAGCGCTTAGCGGCGCGCCTTGA
GGCTCTCAAGGAGAACGGCGGGGCCCGCCTGGCCGAGTACCACGCCAAGG
CCACCGAGCATCTGAGCACGCTCAGCGAGAAGGCCAAGCCGGCGCTCGAG
GACCTGCGCCAAGGCCTGCTGCCGGTGCTGGAGAGCTTCAAGGTCAGCTT
CCTGAGCGCTCTGGAAGAGTACACTAAGAAGCTTAACACCCAGGGTGATC
ATCATCATCATCATCATTAA

aNP formulation: All phospholipids were purchased from Avanti Polar Lipids Inc. For discoidal lipid nanoparticles: from stock solutions (10 mg/mL) in chloroform, DMPC (128 μL), cholesterol (Sigma-Aldrich) (7.5 μL) and for spherical lipid nanoparticles: POPC (66.5 μL), PHPC (17.5 μL), cholesterol (4.5 μL), and tricaprylin (Sigma-Aldrich) (2.8 μL from 0.956 g/mL stock) were combined in a glass vial and dried under vacuum. The resulting film was redissolved in an acetonitrile/methanol mixture (95:5 weight %, 900 μL total volume). Separately, a solution of VHHCD8-apolipoprotein A1 protein in PBS (6.5 mL, 0.14 mg/mL) was prepared twice. Both solutions were simultaneously injected using a microfluidic pump fusion 100 (Chemyx Inc) into a Zeonor herringbone mixer (Microfluidic Chipshop) with a flow rate of 0.8 mL/minute for the lipid solution and a rate of 6 mL/minute for the apolipoprotein A1 solution. The obtained solution was concentrated by centrifugal filtration using either a 10 kDa MWCO for discoidal and a 50 kDa MWCO for spherical aNPs Vivaspin tube at 4000 rpm to obtain a volume of 1 mL. PBS (5 mL) was added, and the solution was concentrated to 1 ml; this was repeated twice. The washed solution was concentrated to approximately 1.5 ml and filtered through a 0.22 μm PES syringe filter to obtain the finished VHHCD8-apolipoprotein A1-containing nanoparticles.

DLS: VHHCD8-apolipoprotein A1-containing nanoparticle formulations in PBS were filtered through a 0.22 μm PES syringe filter and analyzed by dynamic light scattering on a Malvern Zetasizer Nano ZS analyzer. Values are reported as the mean number average size distribution.

Cryo-TEM: First, the surface of 200-mesh lacey carbon supported copper grids (Electron Microscopy Sciences) was plasma treated for 40 seconds using a Cressington 208 carbon coater. Subsequently, 3 μl of VHHCD8-apolipoprotein A1-containing nanoparticles (˜1 mg protein/ml) was applied on a grid and vitrified into a thin film by plunge vitrification in liquid ethane by using an automated robot (FEI Vitrobot Mark IV). Cryo-TEM imaging was performed on the cryoTITAN (Thermo Fisher Scientific), equipped with a field emission gun (FEG), a post-column Gatan imaging filter (model 2002) and a post-GIF 2k x 2k Gatan CCD camera (model 794). The images were acquired at 300 kV acceleration voltage in bright-field TEM mode with zero-loss energy filtering at either 6,500× (dose rate of 1.64 electrons/A2·s) or 24,000× magnification (dose rate of 11.8 electrons/A2·s), and 1s acquisition time.

In vitro binding of VHHCD8-apoA1 in mice splenocytes:

Spleens were obtained from mice, cut into pieces, and strained through a 70 μm strainer (Corning) multiple times to obtain a splenocyte suspension. Cells were spun down at 1500 rpm for 10 minutes, the supernatant was removed and the cells were dissolved in 2 mL 1× red blood cell lysis buffer (Thermofisher). Suspension was incubated at room temperature for 5 minutes, 10 mL Roswell Park Memorial Institute (RPMI) medium (Thermofisher) was added, and cells were again spun down at 1500 rpm for 10 minutes. Cells were then redissolved in RPMI medium and plated in a 96 wells plate at 150.000 cells/well.

The proteins were labeled by adding sulfo-cyanine5-maleimide (Lumiprobe) in dimethyl sulfoxide (DMSO) at a 5× molar excess. This mixture was incubated at room temperature for 2 hours. Excess dye was removed using a PD minitrap G-25 desalting column (Cytiva). Fluorescently labeled aNPs were formulated by adding 6.4 μg Dil for discoidal formulations and 21 μg Dil for spherical formulations. Fluorescently labeled fusion proteins or aNPs and controls were added to the wells and incubated for 30 minutes at 4° C. (for proteins) or 37° C. (for aNPs) after which cells were harvested, washed, and stained for CD3 and CD4 and measured on Cytoflex (Beckman Coulter Inc.). Flow cytometry data was analyzed using FlowJo software (BD).

### Results

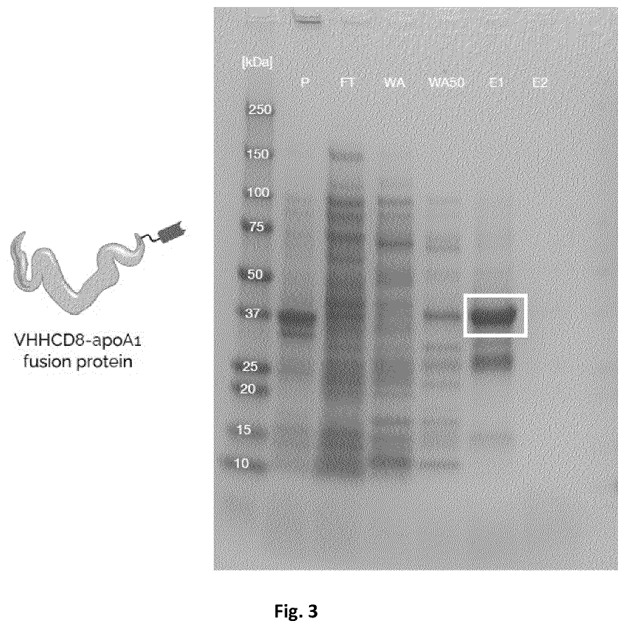

The VHHCD8-apoA 1 fusion protein was successfully expressed in Clearcoli cells. Minor protein contaminants were present after IMAC purification [lane E1] (FIG. 3). The most prominent band corresponds to the fusion protein with a molecular weight of 43.3 kDa (FIG. 3). The correct mass was later confirmed via mass spectrometry (data not shown). Discoidal apolipoprotein nanoparticles were formulated incorporating VHHCD8-apoA1. Using Dynamic Light Scattering (DLS), the particles' size and poly dispersity index (PDI) were determined. The size of the particles remained stable for 7 days (data not shown). On day 14 the size had increased slightly (data not shown). The PDI remained stable for 14 days (data not shown). Cryo-TEM images of the nanoparticles showed the expected discoidal shape (data not shown)

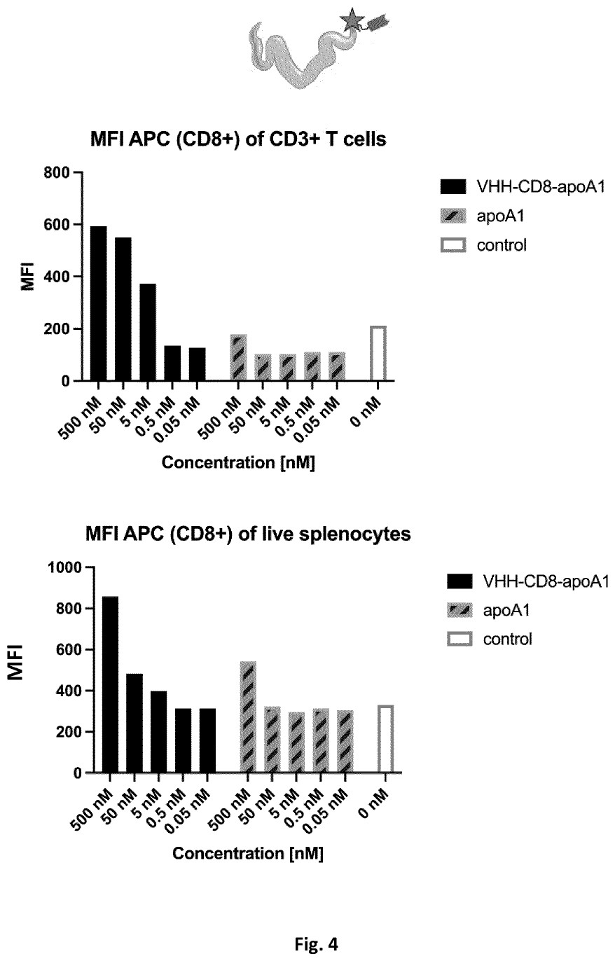

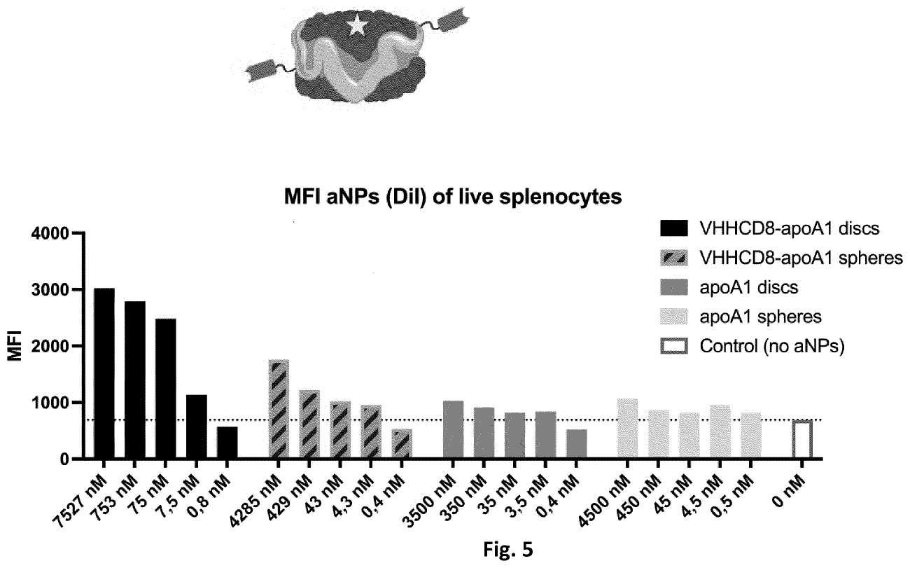

VHHCD8-apoA1 and apoA1 were fluorescently labeled, and subsequently added to mouse splenocytes. For the VHHCD8-apoA 1 fusion protein, a dose dependent increase of Mean Fluorescence Intensity (MFI) was observed, indicating the binding of the fusion protein to the CD8 receptor (FIG. 4; lower panel). The apoA1 condition did not show this dose dependent behavior and had a similar MFI to the control sample. Discoidal and spherical aNPs were formulated with VHHCD8-apoA1 and apoA1. A fluorescent dye was integrated in the lipid structure of the particle. Mouse splenocytes were incubated with the nanoparticles. Both VHHCD8-apoA1 particles showed a dose dependent increase of MFI, indicating the binding of the nanoparticle to the CD8 receptor (FIG. 5). The discoidal nanoparticles showed a greater increase than the spherical nanoparticles. No increase in MFI was observed for the apoA1 nanoparticle conditions.

### Example 5. In Vivo Biodistribution of Lipid Nanoparticles Comprising a Fusion Protein of an Apolipoprotein Fused to a Rerouting Protein or Peptide

### Materials and Methods

### Formulation of Spherical VHHCD8-aNPs

All phospholipids were purchased from Avanti Polar Lipids Inc. For spherical aNPs, POPC (66.5 μL), PHPC (17.5 μL), cholesterol (4.5 μL), and tricaprylin (Sigma-Aldrich) (2.8 μL from 0.956 g/mL stock) were combined in a glass vial and dried under vacuum. The resulting film was redissolved in an acetonitrile/methanol mixture (95: 5%, 900 L total volume). Separately, a solution of VHHCD8-apoA1 protein (SEQ ID NO:1) in PBS (6.5 mL, 0.14 mg/mL) was prepared twice.

Both solutions were simultaneously injected using a microfluidic pump fusion 100 (Chemyx Inc) into a Zeonor herringbone mixer (Microfluidic Chipshop) with a flow rate of 0.8 mL/minute for the lipid solution and a rate of 6 mL/minute for the apoA1 solution. The obtained solution was concentrated by centrifugal filtration using a 50 kDa MWCO for spherical aNPs Vivaspin tube at 4000 rpm to obtain a volume of 1 mL. PBS (5 mL) was added, and the solution was concentrated to 1 ml; this was repeated twice. The washed solution was concentrated to approximately 1.5 ml and filtered through a 0.22 μm PES syringe filter to obtain the finished aNPs.

Table 1. VHHCD8-apolipoprotein A1 fusion protein nanoparticle composition in molar percentages. DIR: 1,1-dioctadecyl-3,3,3,3-tetramethylindotricarbocyanine; Lyso-PC: 1-palmitoyl-2-hydroxy-sn-glycero-3-phosphocholine; POPC: 1-palmitoyl-2-oleoyl-sn-glycero-3-phosphocholine.

| | Mol % |
|---|---|
| POPC | 12.4% |
| Lyso-PC | 4.9% |
| Triglycerides | 76.1% |
| Cholesterol | 4.3% |
| DiR | 2% |
| Protein | 0.3% |

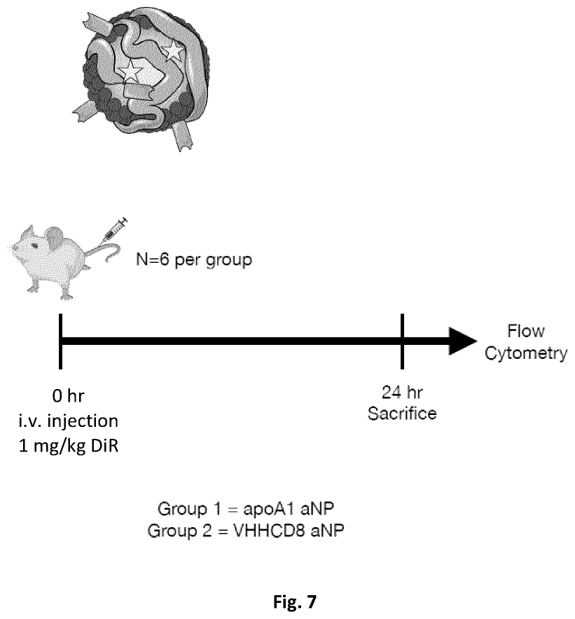

### In Vivo Biodistribution of VHHCD8-apoA1 in Mice (FIG. 7)

Spherical VHHCD8-aNPs with 2 mol % DiR (1,1′-dioctadecyl-3,3,3′,3′-tetramethylindotricarbocyanine iodide) were intravenously injected in C57BL/6 mice at a dose of 1 mg/kg DiR. After 24 hours, mice were sacrificed, blood was harvested and after PBS perfusion other tissues of interest (bone marrow, spleen, and lymph nodes) were harvested. The tissues were homogenized into a single cell suspension, stained for myeloid (CD115, F4/80, Ly6C, CD11b, CD45, Ly6G, CD11c), lymphoid (CD45, CD11b, CD3, CD19, CD4, CD8a), and progenitor (CD117, CD34, Ly-6A/E, CD135, CD16/32, CD48, CD41, CD150, CD3, CD11b, CD45R/B220, Ly-76, Ly6G, Ly6C) cell markers, and analyzed on Cytoflex (Beckman Coulter Inc.). Flow cytometry data was analyzed using FlowJo software (BD).

### Results

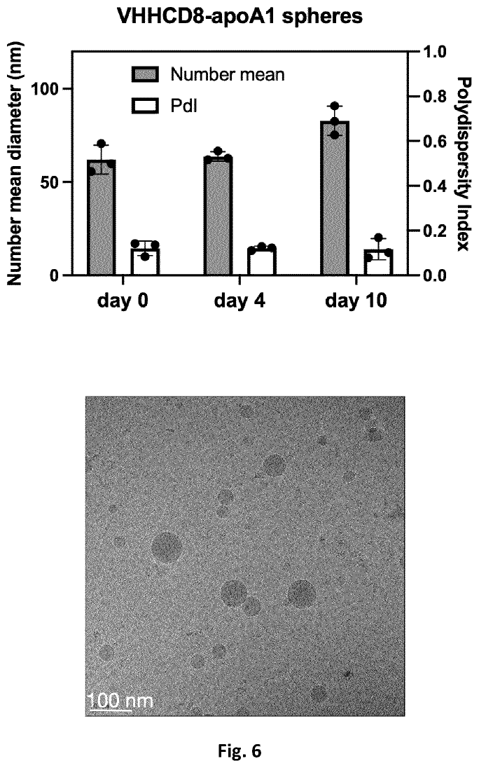

The spherical lipid nanoparticles containing VHHCD8-apolipoprotein A1 were characterized using DLS to determine their hydrodynamic size (number mean) and polydispersity index (Pdi), and cryogenic electron microscopy was used to determine their size and morphology (FIG. 6).

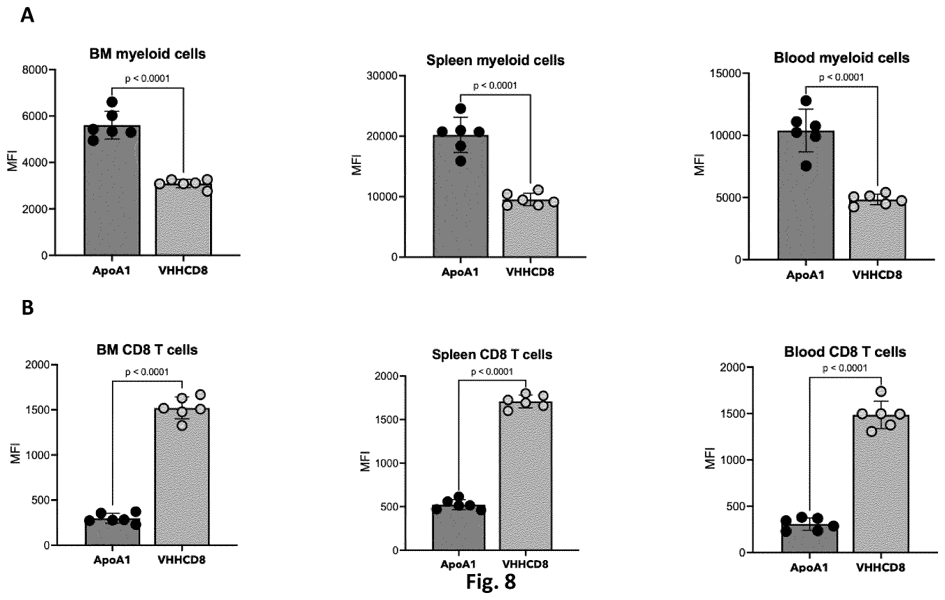

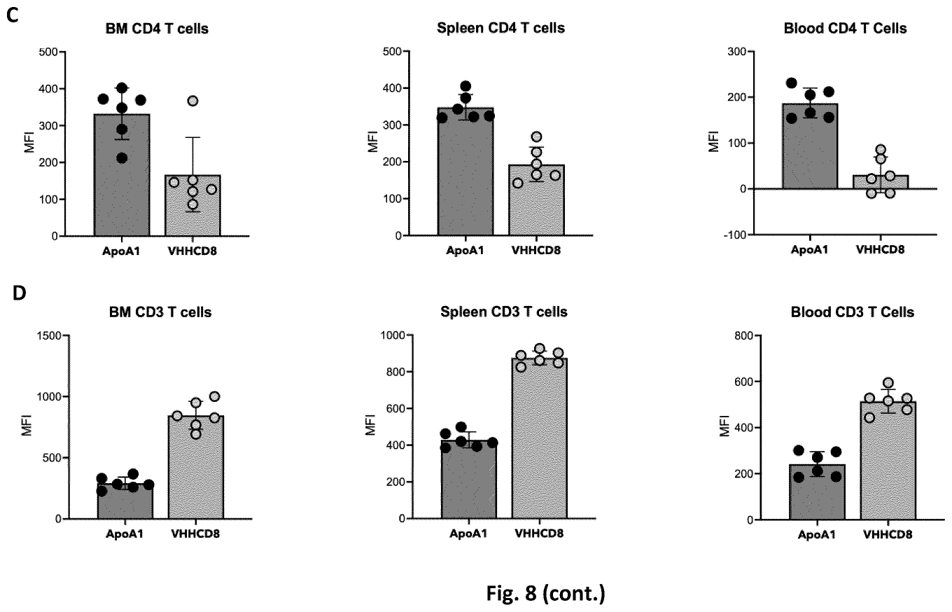

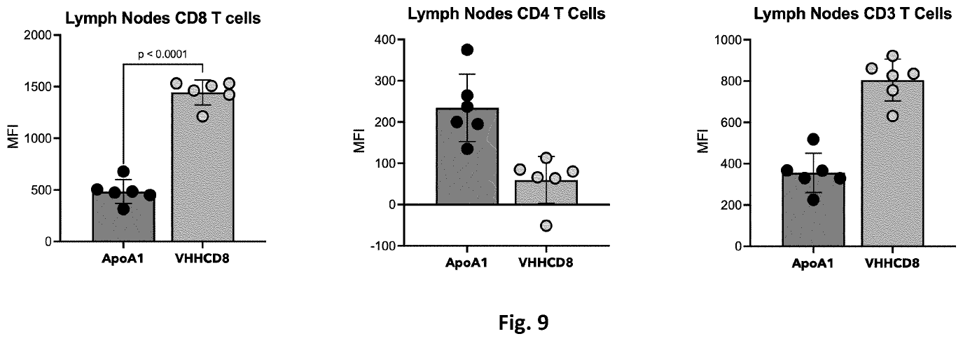

The in vivo biodistribution data show that VHHCD8-apolipoprotein A1 fusion protein nanoparticles associated significantly less with myeloid cells in the bone marrow, spleen, and blood when compared with control apolipoprotein A1 protein nanoparticles (FIG. 8A). VHHCD8-apolipoprotein A1 fusion protein nanoparticles associated significantly more with CD8+ T cells in the bone marrow, spleen, and blood when compared with control apolipoprotein A1 protein nanoparticles (FIG. 8B). VHHCD8-apolipoprotein A1 fusion protein nanoparticles associated less with CD4+ T cells in the bone marrow, spleen, and blood when compared with control apolipoprotein A1 protein nanoparticles (FIG. 8C). In addition, VHHCD8-apolipoprotein A1 fusion protein nanoparticles associated more with CD3+ T cells in the bone marrow, spleen, and blood when compared with control apolipoprotein A1 protein nanoparticles (FIG. 8D). In lymph nodes, VHHCD8-apolipoprotein A1 fusion protein nanoparticles associated more with CD8+ or CD3+when compared with control apolipoprotein A1 protein nanoparticles, and VHHCD8-apolipoprotein A1 fusion protein nanoparticles associated less with CD4+ T cells in the lymph nodes when compared with control apolipoprotein A1 protein nanoparticles (FIG. 9).

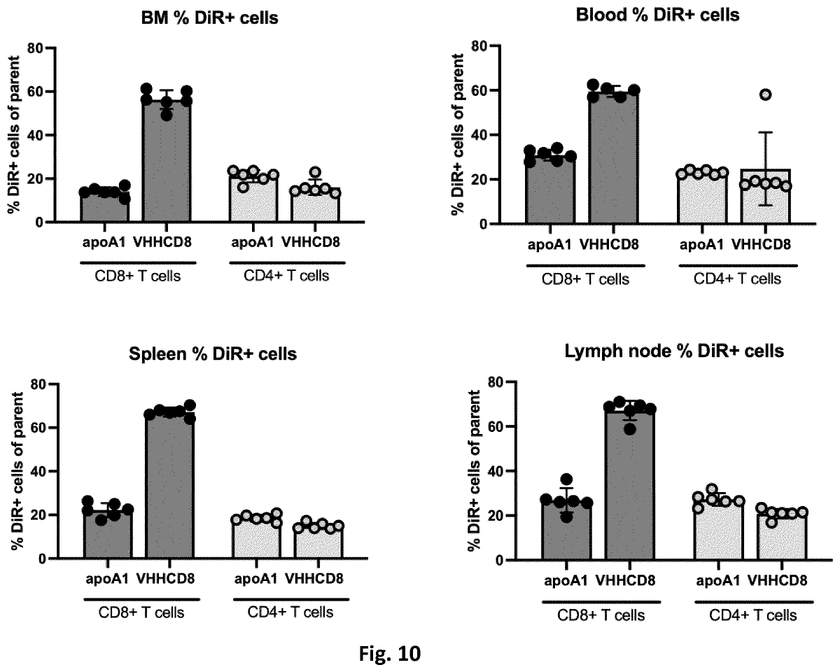

VHHCD8-apolipoprotein A1 fusion protein nanoparticles associated with more CD8+ T cells in the bone marrow, blood, spleen, and lymph nodes when compared with control apolipoprotein A1 protein nanoparticles (FIG. 10). There was no difference in CD4+ T cell engagement between tretament groups (FIG. 10).

### Example 6. Lipid Nanoparticles Comprising Fusion Proteins of an Apolipoprotein Fused to a Rerouting Peptide or Protein and/or Fusion Proteins of an Apolipoprotein Fused to an Immunomodulatory Biomolecule

### Materials and Methods

ApoA1-IL-2 was reduced to break disulfide bonds between the cysteines in the protein by incubation in 1 mM DTT at room temperature for 2 hours. ApoA1-IL-2 and VHHCD8-apoA1 were fluorescently labeled with cy3 and cy5, respectively, as described in Example 4.

The apoA1-IL-2 fusion protein has the sequence as defined by SEQ ID NO: 3 or 5, or is encoded by a sequence as defined by SEQ ID NO: 4 or 6.

| SEQ | |
|---|---|
| ID | |
| NO: | Sequence |
| 3 | MDDPPQSPWDRVKDLATVYVDVLKDSGRDYVSQFEGSALGKQLNLKLLDNWDSVTSTF |
| | SKLREQLGPVTQEFWDNLEKETEGLRQEMSKDLEEVKAKVQPYLDDFQKKWQEEMELY |
| | RQKVEPLRAELQEGARQKLHELQEKLSPLGEEMRDRARAHVDALRTHLAPYSDELRQRL |
| | AARLEALKENGGARLAEYHAKATEHLSTLSEKAKPALEDLRQGLLPVLESFKVSFLSALEE |
| | YTKKLNTQGSGSGSGSGSGTAPTSSSTKKTQLQLEHLLLDLQMILNGINNYKNPKLTRML |
| | TFKFYMPKKATELKHLQCLEEELKPLEEVLNLAQSKNFHLRPRDLISNINVIVLELKGSETT |
| | FMCEYADETATIVEFLNRWITFCQSIISTLTGSGSGSGSGSGASHHHHHH |
| 4 | ATGGATGATCCACCTCAAAGTCCATGGGATCGCGTGAAGGACCTGGCCACTGTGTAC |
| | GTGGATGTGCTCAAAGACAGCGGCAGAGACTATGTGTCTCAGTTTGAAGGATCCGCC |
| | TTGGGCAAACAATTGAACCTTAAGCTGCTGGACAACTGGGACAGCGTGACGTCCACC |
| | TTCAGCAAGCTGCGCGAACAGCTCGGCCCTGTGACCCAGGAATTCTGGGATAACCTG |
| | GAAAAGGAGACAGAGGGCCTGCGCCAGGAGATGAGCAAGGATCTGGAGGAGGTGAA |
| | GGCCAAGGTGCAGCCGTACCTGGACGACTTCCAGAAGAAGTGGCAGGAGGAAATGG |
| | AGTTATATCGCCAAAAGGTCGAGCCGCTGCGCGCAGAGCTGCAGGAGGGCGCGCGC |
| | CAGAAGCTGCACGAGCTGCAAGAGAAGCTGAGCCCATTAGGTGAAGAAATGCGTGA |
| | CCGCGCGCGCGCACATGTCGACGCATTACGCACACATTTAGCGCCGTACTCTGACG |
| | AGCTGCGCCAGCGCTTAGCGGCGCGCCTTGAGGCTCTCAAGGAGAACGGTGGAGCT |
| | CGCTTAGCAGAATATCACGCCAAGGCCACCGAGCATCTGAGCACGCTCAGCGAGAA |
| | GGCCAAGCCGGCGCTCGAGGACCTGCGCCAAGGCCTGCTGCCGGTGCTGGAGAGC |
| | TTCAAGGTCAGCTTCCTGAGCGCTCTGGAAGAGTACACTAAGAAGCTTAACACCCAG |
| | GGTTCTGGTAGCGGCTCTGGTTCTGGTTCTGGTACCGCGCCGACCAGCAGCAGCAC |
| | CAAGAAGACCCAGCTGCAACTGGAACACCTGCTGCTGGACCTGCAAATGATTCTGAA |
| | CGGTATCAACAACTACAAGAACCCGAAACTGACCCGTATGCTGACCTTCAAGTTTTAT |
| | ATGCCGAAGAAAGCGACCGAGCTGAAGCACCTGCAATGCCTGGAGGAAGAGCTGAA |
| | ACCGCTGGAAGAGGTGCTGAACCTGGCGCAAAGCAAGAACTTCCACCTGCGTCCGC |
| | GTGACCTGATCAGCAACATTAACGTGATCGTTCTGGAACTGAAAGGCAGCGAGACCA |
| | CCTTTATGTGCGAATATGCGGATGAGACCGCGACCATTGTTGAGTTCCTGAACCGTT |
| | GGATCACCTTTTGCCAGAGCATCATTAGCACCCTGACCGGCTCTGGTAGCGGCTCTG |
| | GTTCTGGTTCTGGTGCTAGCCATCACCATCACCATCATTAA |
| 5 | MDDPPQSPWDRVKDLATVYVDVLKDSGRDYVSQFEGSALGKQLNLKLLDNWDSVTSTF |
| | SKLREQLGPVTQEFWDNLEKETEGLRQEMSKDLEEVKAKVQPYLDDFQKKWQEEMELY |
| | RQKVEPLRAELQEGARQKLHELQEKLSPLGEEMRDRARAHVDALRTHLAPYSDELRQRL |
| | AARLEALKENGGARLAEYHAKATEHLSTLSEKAKPALEDLRQGLLPVLESFKVSFLSALEE |
| | YTKKLNTQGSGSGSGSGSGTAPTSSSTKKTQLQLEHLLLDLQMILNGINNYKNPKLTRML |
| | TAKFAMPKKATELKHLQCLEEELKPLEEVLNGAQSKNFHLRPRDLISNINVIVLELKGSETT |
| | FMCEYADETATIVEFLNRWITFAQSIISTLTGSGSGSGSGSGASHHHHHH |
| 6 | ATGGATGATCCACCTCAAAGTCCATGGGATCGCGTGAAGGACCTGGCCACTGTGTAC |
| | GTGGATGTGCTCAAAGACAGCGGCAGAGACTATGTGTCTCAGTTTGAAGGATCCGCC |
| | TTGGGCAAACAATTGAACCTTAAGCTGCTGGACAACTGGGACAGCGTGACGTCCACC |
| | TTCAGCAAGCTGCGCGAACAGCTCGGCCCTGTGACCCAGGAATTCTGGGATAACCTG |
| | GAAAAGGAGACAGAGGGCCTGCGCCAGGAGATGAGCAAGGATCTGGAGGAGGTGAA |
| | GGCCAAGGTGCAGCCGTACCTGGACGACTTCCAGAAGAAGTGGCAGGAGGAAATGG |
| | AGTTATATCGCCAAAAGGTCGAGCCGCTGCGCGCAGAGCTGCAGGAGGGCGCGCGC |
| | CAGAAGCTGCACGAGCTGCAAGAGAAGCTGAGCCCATTAGGTGAAGAAATGCGTGA |
| | CCGCGCGCGCGCACATGTCGACGCATTACGCACACATTTAGCGCCGTACTCTGACG |
| | AGCTGCGCCAGCGCTTAGCGGCGCGCCTTGAGGCTCTCAAGGAGAACGGTGGAGCT |
| | CGCTTAGCAGAATATCACGCCAAGGCCACCGAGCATCTGAGCACGCTCAGCGAGAA |
| | GGCCAAGCCGGCGCTCGAGGACCTGCGCCAAGGCCTGCTGCCGGTGCTGGAGAGC |
| | TTCAAGGTCAGCTTCCTGAGCGCTCTGGAAGAGTACACTAAGAAGCTTAACACCCAG |
| | GGTTCTGGTAGCGGCTCTGGTTCTGGTTCTGGTACCGCGCCGACCAGCAGCAGCAC |
| | CAAGAAGACCCAGCTGCAACTGGAACACCTGCTGCTGGACCTGCAAATGATTCTGAA |
| | CGGTATCAACAACTACAAGAACCCGAAACTGACCCGTATGCTGACCGCGAAGTTTgct |
| | ATGCCGAAGAAAGCGACCGAGCTGAAGCACCTGCAATGCCTGGAGGAAGAGCTGAA |
| | ACCGCTGGAAGAGGTGCTGAACgggGCGCAAAGCAAGAACTTCCACCTGCGTCCGCG |
| | TGACCTGATCAGCAACATTAACGTGATCGTTCTGGAACTGAAAGGCAGCGAGACCAC |
| | CTTTATGTGCGAATATGCGGATGAGACCGCGACCATTGTTGAGTTCCTGAACCGTTG |
| | GATCACCTTTgccCAGAGCATCATTAGCACCCTGACCGGCTCTGGTAGCGGCTCTGGT |
| | TCTGGTTCTGGTGCTAGCCATCACCATCACCATCATTAA |

Discoidal aNPs were formulated with 513 μg apoA1-IL-2 and 467 μg VHHCD8-apoA1. Spherical aNPs were formulated in two sizes, where only the triglyceride content was varied. Big spheres were formulated as described in Example 5, small spheres were formulated with half the triglyceride content of the big spheres. Spheres with only apoA1-IL-2 (donor) contained 1025 μg apoA1-IL-2. Spheres with only VHHCD8-apoA1 (acceptor) contained 921 μg VHHCD8-apoA1. Big and small spheres with a 1:1 mix of donor and acceptor contained 513 μg apoA1-IL-2 and 467 μg VHHCD8-apoA1.

The aNPs in PBS were pipetted into a black 96 well plate (Thermo Fisher Scientific-Nunclon) at 100 μL per well. For the 1:1 mix of donor only spheres and acceptor only spheres, 50 μL of each formulation was put in the well. A 1:1 mix (molar ratio) of fluorescently labeled apoA1-IL-2 and VHHCD8-apoA1 was included as control. The samples were excited at 520 nm and emission was detected between 560 and 780 nm with a step size of 5 nm in a Spark plate reader (Tecan). Data was normalized by dividing the intensity of one sample on each wavelength by the intensity of that sample at 565 (donor emission maximum).

### Results

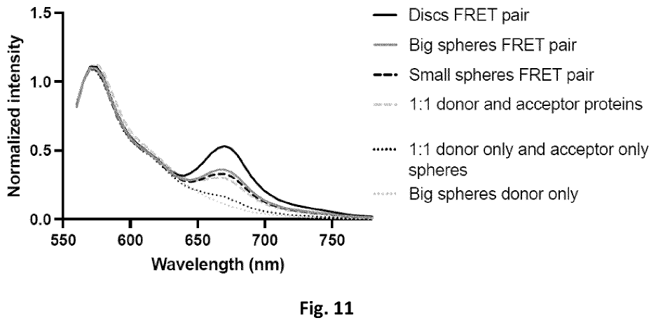

The FRET results indicate that both VHHCD8-apolipoprotein A1 and IL-2-apolipoprotein A1 fusion proteins can be readily and stably incorporated into one lipid nanoparticle formulation (FIG. 11).

### Example 6. Production, Analysis, and Evaluation of CD8+ T Cell Targeted Apolipoprotein Nanoparticles Containing mRNA

### Materials and Methods

### Apolipoprotein Nanoparticle Production

CD8-aNP-mRNA are prepared by rapid mixing through a T-junction mixer. The cationic ionizable lipid ALC-0315, dimyristoylfosfatidylcholine (DMPC), cholesterol, and tricaprylin are dissolved in ethanol at appropriate molar ratios (Table 2) to a final concentration of 10 mM total lipid. mRNA is dissolved in 25 mM sodium acetate buffer at pH 4.0 to obtain a final mixture with a defined nucleic acid to lipid weight/umol ratio of 0.0278 (N/P 6). In the first mixing step, the organic and aqueous solutions are mixed at a flow ratio of 1:3 (v: v) and a total flow rate of 28 mL/min. The resulting mixture is dialyzed against a 1000-fold volume of phosphate-buffered saline (PBS) pH 7.4 overnight. In a second mixing step, the lipid nanoparticles containing mRNA and CD8VHH-apolipoprotein A1 fusion protein are mixed at a flow ratio of 1:3 (v: v) and a total flow rate of 28 mL/min. The resulting CD8-aNP-mRNA are sterile filtered (0.2 μm) and concentrated using centrifugal flow filtration.

TABLE 2
CD8-aNP-mRNA composition (molar ratios)

| | ALC- | | CD8- | | |
|---|---|---|---|---|---|
| DMPC | 0315 | Cholesterol | apolipoprotein | mRNA | Tricaprylin |
| 9 | 19 | 21 | 0.08 | 0.03 | 51 |

### Apolipoprotein Nanoparticle Physicochemical Analysis

CD8-aNP-mRNA are characterized according to size distribution by dynamic light scattering, zeta potential by laster doppler electrophoresis, mRNA encapsulation efficiency by Ribogreen assay, and cholesterol/phospholipid content using dedicated colorimetric assays.

Apolipoprotein nanoparticle evaluation ex vivo

Spleens are obtained from mice, cut into pieces, and strained through a 70 μm strainer (Corning) multiple times to obtain a splenocyte suspension. Cells are spun down at 1500 rpm for 10 minutes, the supernatant is removed, and the cells are dissolved in 2 mL 1×red blood cell lysis buffer (Thermofisher). Cell suspension is incubated at room temperature for 5 minutes, 10 mL RPMI medium (Thermofisher) is added, and cells are again spun down at 1500 rpm for 10 minutes. Cells are then redissolved in RPMI medium and plated in a 96 wells plate at 150.000 cells/well. CD8-aNP-mRNA encoding for mCherry and controls formulations are added to the wells in appropriate concentrations and incubated for 6 hours 37° C. after which cells are harvested, washed, and stained for CD3 and CD4 and measured on Cytoflex (Beckman Coulter Inc.). Flow cytometry data is analyzed using FlowJo software (BD).

### Apolipoprotein Nanoparticle Evaluation In Vivo

CD8-aNP-mRNA encoding for mCherry and control formulations are intravenously injected in C57BL/6 mice at a dose of 0.5 mg/kg mRNA. After 6 hours, mice are sacrificed, blood is harvested and after PBS perfusion other tissues of interest (bone marrow, spleen, and lymph nodes) are harvested. The tissues are homogenized into a single cell suspension, stained for myeloid (CD115, F4/80, Ly6C, CD11b, CD45, Ly6G, CD11c), lymphoid (CD45, CD11b, CD3, CD19, CD4, CD8a), and progenitor (CD117, CD34, Ly-6A/E, CD135, CD16/32, CD48, CD41, CD150, CD3, CD11b, CD45R/B220, Ly-76, Ly6G, Ly6C) cell markers, and analyzed on Cytoflex (Beckman Coulter Inc.). Flow cytometry data is analyzed using FlowJo software (BD).

### Results

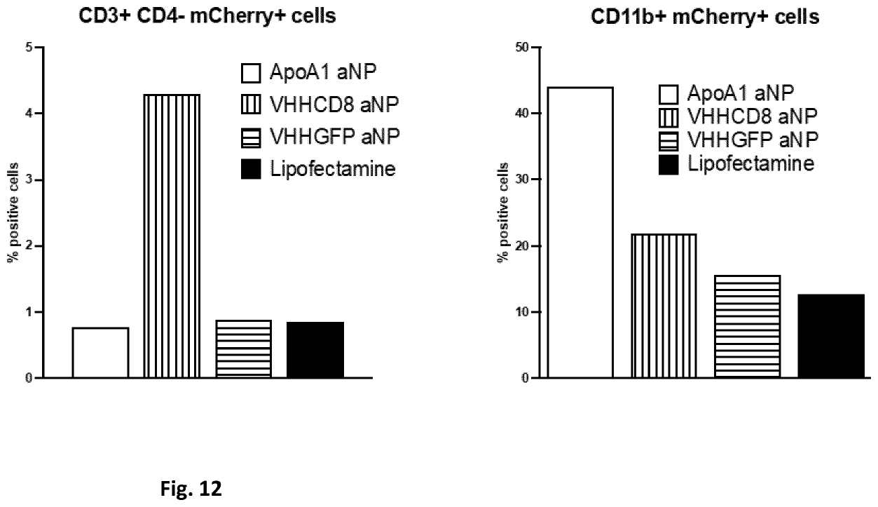

The flow cytometry analysis of mouse splenocytes incubated ex vivo with apoA1 aNP, VHHCD8-apoA1 aNP, and VHHGFP-apoA1 aNP containing mRNA encoding for fluorescent mCherry protein is shown in FIG. 12 (left panel: CD3+ CD4-mCherry+ T cells, right panel: CD11b+mCherry+ myeloid cells). The results show that VHHCD8-apoA1 aNP induce functional mCherry reporter gene expression in significantly more CD3+ CD4-T cells when compared to apoA1 aNP, VHHGFP-apoA1 aNP and lipofectamine controls. ApoA1 aNP induce functional mCherry reporter gene expression in significantly more CD11b+ myeloid cells when compared to VHHCD8-apoA1 aNP, VHHGFP-apoA1 aNP and lipofectamine controls. These results indicate that apoA1 aNP have a natural propensity to interact with myeloid cells, and that these aNPs can be rerouted to other immune cells (such as CD8+ T cells) by incorporating fusion proteins of apolipoproteins and targeting moieties.

1. A modified apolipoprotein comprising an apolipoprotein, apolipoprotein derivative, apolipoprotein mimetic or apolipoprotein mimetic derivative attached to a targeting body, wherein the targeting body is a molecule capable of binding a molecule on the cell surface of a target cell.

2. The modified apolipoprotein according to claim 1, wherein the targeting body is selected from:
an antibody or antigen binding fragment of an antibody,
a protein ligand, a protein binding domain, or a protein binding fragment thereof,
a peptide,
a peptidometic, or
a sugar polymer.

3. The modified apolipoprotein according to claim 2, wherein the antibody or antigen binding fragment thereof is selected from a Fab, a Fab2, a scFv, a scFv-Fc, a dAb-Fc, a free light chain antibody, a half antibody, a bispecific Fab2, a Fab3, a trispecific Fab3 a diabody, a bispecific diabody, a triabody, a trispecific triabody, a minibody, an IgG, an IgNAR, a monovalent IgG, a VhH, a nanobody, or a VNAR, or an antigen binding fragment thereof, preferably a camelid or shark VhH or derivative thereof or antigen binding fragment thereof.

4. The modified apolipoprotein according to claim 1, wherein the targeting body binds a protein on the surface of a lymphoid cell, a myeloid cell, a tumor cell, an endothelial cell, a hematopoietic stem and progenitor cell (HSPC), a hematopoietic stem cell (HSC), a multipotent progenitor (MPP), a common myeloid progenitor cell (CMP), or wherein the targeting body binds a bacterial, viral, fungal or parasitic protein or antigen,
preferably wherein the lymphoid or myeloid cell is selected from a monocyte, a macrophage, an MI-like macrophage, an M2-like macrophage, an eosinophil, a basophil, a mast cell, an NK cell, a B cell, a plasma cell, a regulatory T cell, a hematopoietic stem cell, a granulocyte, a T helper cell such as Th1, Th2, Th17 or Th22, a dendritic cell, such as a plasmacytoid dendritic cell, a conventional DC 1 or a conventional DC 2, or a tumor-associated macrophage.

5. The modified apolipoprotein according to claim 1, wherein the targeting body binds a protein selected from CDla, CD1b, CD1c, CD1d, CD2, CD3, CD4, CD5, CD6, CD7, CD8, CD9, CD10, CD11a, CD11b, CD11c, CD11d, CDw12, CD13, CD14, CD15, CD15s, CD15u, CD16, CDw17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42a, CD42b, CD42c, CD42d, CD43, CD44, CD45, CD45RO, CD45RA, CD45RB, CD46, CD47, CD48, CD49a, CD49b, CD49c, CD49d, CD49e, CD49f, CD50, CD51, CD52, CD53, CD54, CD55, CD56, CD57, CD58, CD59, CD60a, CD60b, CD60c, CD61, CD62E, CD62L, CD62P, CD63, CD64, CD65, CD66a, CD66b, CD66c, CD66d, CD66e, CD66f, CD68, CD69, CD70, CD71, CD72, CD73, CD74, CD75, CD75s, CD77, CD79a, CD79B, CD80, CD81, CD82, CD83, CDw84, CD85, CD86, CD87, CD88, CD89, CD90, CD91, CD92, CD93, CD94, CD95, CD96, CD97, CD98, CD99, CD100, CD101, CD102, CD103, CD104, CD105, CD106, CD107a, CD107b, CD108, CD109, CD110, CD111, CD112, CD114, CD115, CD116, CD117, CD118, CD119, CD120a, CD120b, CD121a, CDw121b, CD122, CD123, CD124, CD125, CD126, CD127, CDw128, CD129, CD130, CDw131, CD132, CD133, CD134, CD135, CDw136, CDw137, CD138, CD139, CD140a, CD140b, CD141, CD142, CD143, CD144, CD145, CD146, CD147, CD148, CD150, CD151, CD152, CD153, CD154, CD155, CD156a, CD156b, CD157, CD158, CD158a, CD158b, CD159a, CD160, CD161, CD162, CD162R, CD163, CD164, CD165, CD166, CD167a, CD168, CD169, CD170, CD171, CD172a, CD173, CD174, CD175, CD175s, CD176, CD177, CD178, CD179a, CD179b, CD180, CD183, CD184, CD195, CDw197, CD200, CD201, CD202b, CD203c, CD204, CD205, CD206, CD207 (CLEC4K), CD208, CD209 (CLEC4L), CDw210, CD212, CD213al, CD213a2, CDw217, CD220, CD221, CD222, CD223, CD224, CD225, CD226, CD227, CD228, CD229, CD230, CD231, CD232, CD233, CD234, CD235a, CD235b, CD236, CD236R, CD238, CD239, CD240CE, CD240D, CD241, CD242, CD243, CD244, CD245, CD246, CD247, CXCR1, CXCR2, CXCR3, CXCR4, CXCR5, CXCR6, CXCR7, CCR1, CCR2, CCR3, CCR4, CCR5, CCR7, CCR8, CCR9, CLECIA, CLECIB, CLEC2A, CLEC2B, CLEC3A, CLEC3B, CLEC4A, CLEC4C (CD303), CLEC4D, CLEC4J CLEC4E, CLEC4F, CLEC4G, ASGR1 (CLEC4H1), ASGR2 (CLEC4H2), FCER2 ( ) CLEC4M, CLEC5A, CLEC6A, CLEC7A, OLRI (CLEC8A), CLEC9A, CLEC10A, CLEC11A, CLEC12A, CLEC12B, CD302 (CLEC13A), LY75 (CLEC13B), PLA2R₁ (CLEC13C), MRC1 (CLEC13D), MRC2 (CLEC13E), CLEC14A, CLEC16A, CLEC17A, KLRAI, KLRB1 (CLEC5B), KLRC1, KLRC2, KLRC3, KLRC4, KLRD1, KLRF1 (CLEC5C), KLRG1 (CLEC15A), KLRG2 (CLEC15B), KLRK1, AGC1, ATRNL1, BCAN, CD248, CHODL, CL-K1-Ia, CL-K1-Ib, CL-K1-Ic, CLECSF5, COLEC10, COLEC11, COLEC12, CSPG3, FCER2, FREM1, HBXBP, LAYN, LOC348174, LOC728276, MAFA, MBL2, MGC34761, MICL, MRCIL1, PAP, PKD1, PKDIL2, PRG2, PRG3, REGIA, REGIB, REG3A, REG3G, REG4, SELE, SELL, SELP, SFTPA1, SFTPA2, SFTPA2B, SFTPD, SRCL, THBD, VCAN, Alphafetoprotein (AFP), Carcinoembryonic antigen (CEA), CA-125, MUC-1, Epithelial tumor antigen (ETA), Tyrosinase, and melanoma-associated antigen (MAGE).
preferably selected from CD14, CD11b, CD357 (GITR), CD193, CD123, CD117, CD56, CD19, CD38, CD25, CD133, CXCR3, CCR3, CD196, CCR10, CD64, CD206, CLEC4C (CD303), CLEC9A, CD1c, CD163, Alphafetoprotein (AFP), Carcinoembryonic antigen (CEA), CA-125, MUC-1, Epithelial tumor antigen (ETA), Tyrosinase, and melanoma-associated antigen (MAGE).

6. The modified apolipoprotein according to claim 1, wherein the apolipoprotein, apolipoprotein derivative, apolipoprotein mimetic or apolipoprotein mimetic derivative is selected from apoAl, apoA-1 Milano, apoA2, apoA4, apoA5, apoB, apoB48, apoB100, apoC-I, apoC-II, apoC-III, apoC-IV, apoD, apoE, apoF, apoH, apoL, apoL 1, apoL2, apoL3, apoL4, apoL5, apoL6, apoLD1, apoO, apoOL and apoM, or a combination thereof, or a mimetic or derivative thereof,
preferably selected from apoA1, apoA2, apoA4, apoA5, apoB48, apoB100, apoC-I, apoC-II, apoC-III, apoC-IV, apoD, apoE, apoF, apoH, apoL and apoM or a mimetic or derivative thereof,
more preferably selected from apoAl, apoA2, apoA4, apoA5, apoB100, apoC-I, apoC-II, apoC-III, apoC-IV and apoE or a mimetic or derivative thereof,
even more preferably selected from apoA1, apoA4, apoA5, apoB100, apoC-III and apoE or a mimetic or derivative thereof,
most preferably selected from apoA1, apoB100 and apoE or a mimetic or derivative thereof.

7. A lipid nanoparticle comprising an outer layer and a core, wherein the outer layer comprises:
a phospholipid;
a sterol; and
the modified apolipoprotein as defined in claim 1; and wherein the core comprises at least one core component selected from:
a lipid, a cationic lipid, or a polyvalent molecule.

8. The lipid nanoparticle according to claim 7, wherein the nanoparticle further comprises a payload, preferably wherein the payload is comprised in the core, the phospholipid layer or wherein the payload is bound to a component of the outer layer.

9. The lipid nanoparticle according to claim 8, wherein the payload is selected from a nucleic acid or a nucleic acid analog, a therapeutic, a biologic, a cytokine, a chemokine, a hormone, a growth factor, or combinations thereof.

10. The lipid nanoparticle according to claim 9, wherein the lipid nanoparticle comprises a nucleic acid and a cationic or ionizable cationic lipid.

11. The lipid nanoparticle according to claim 10, wherein the nucleic acid and the cationic or ionizable cationic lipid are comprised by the core, and the modified apolipoprotein and the phospholipid are comprised by the outer layer.

12. A lipid nanoparticle comprising an outer layer and a core, wherein the outer layer comprises:
a phospholipid;
a sterol; and
the modified apolipoprotein as defined in claim 1; and
wherein the core comprises a nucleic acid and a cationic or ionizable cationic lipid.

13. The lipid nanoparticle according to claim 10, wherein the nanoparticle core further comprises a filler, preferably a filler selected from a triacylglyceride and a cholesterol acyl ester, or combinations thereof, such as wherein the triacylglyceride is tricaprylin and/or wherein the cholesterol acyl ester is cholesteryl caprylate and/or cholesteryl oleate.

14. The lipid nanoparticle according to claim 2, wherein the nucleic acid is RNA, DNA or a nucleic acid analogue.

15. The lipid nanoparticle according to claim 14, wherein the RNA is microRNA (miRNA), small interfering RNA (siRNA), piwi-interacting RNA (piRNA), small nuclear RNA (snRNA), small nucleolar RNA (snoRNA), transfer RNA (tRNA), tRNA-derived small RNA (tsRNA), small regulatory RNA (srRNA), messenger RNA (mRNA), modified mRNA, ribosomal RNA (rRNA), long non-coding RNA (lncRNA), or guide RNA (gRNA), or combinations thereof and/or modifications thereof.

16. The lipid nanoparticle according to claim 14, wherein the DNA is single stranded or double stranded DNA.

17. The lipid nanoparticle according to claim 2, wherein the nucleic acid is an antisense oligonucleotide and the antisense oligonucleotide is single strand DNA or RNA consisting of nucleotide or nucleoside analogues containing modifications of the phosphodiester backbone or the 2′ ribose.

18. The lipid nanoparticle according to claim 17, wherein the nucleotide or nucleoside analogues are selected from locked nucleic acid (LNA), bridged nucleic acid (BNA), morpholino or peptide nucleic acid (PNA), glycol nucleic acid (GNA), threose nucleic acid (TNA), hexitol nucleic acids (HNA), or mixtures or combinations thereof.

19. The lipid nanoparticle according to claim 10, wherein the cationic or ionizable cationic lipid is selected from an ionizable cationic ester of a long chain alcohol, an ionizable cationic ester of a diglyceride or an ionizable cationic ester of a sterol or combinations thereof.

20. The lipid nanoparticle according to claim 10, wherein the ionizable cationic lipid is a molecule according to any one of Formulae (I), (II), (III), (IV) or (V) 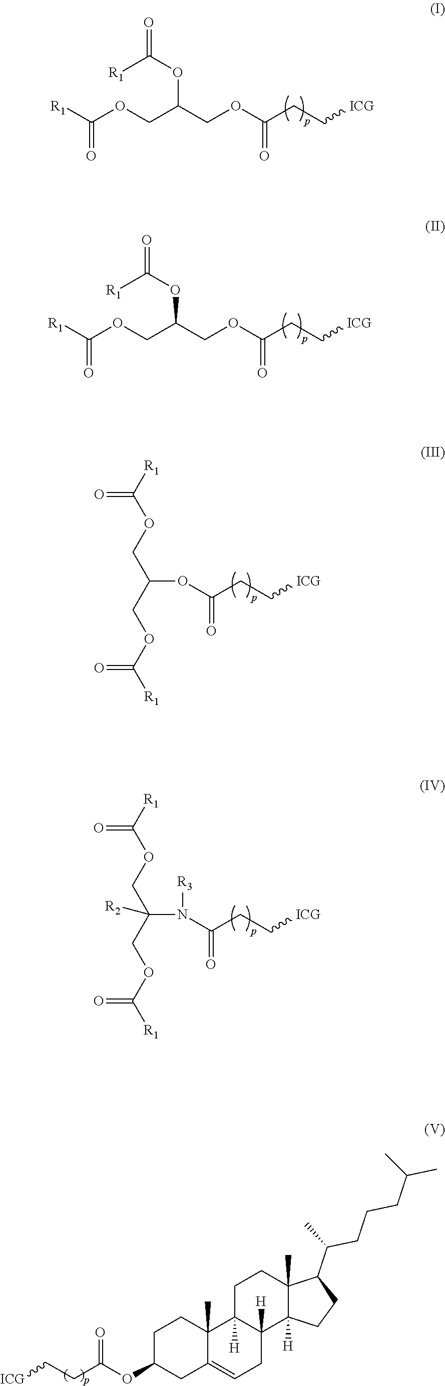
wherein ICG is 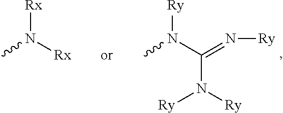
wherein the wavy line indicates the point of attachment to the compound of formulae (I), (II), (III), (IV) or (V);
p is an integer selected from 0, 1, 2, 3, 4, 5, 6, 7, 8, 9, 10 and 11;
each R₁ is independently selected from the group consisting of linear or branched C1-C19 alkyl, linear or branched C1-C19 alkenyl, aryl, arylene-alkyl and alkylene-aryl group, wherein said alkyl or alkenyl group optionally contains up to 5 heteroatoms, independently selected from O and N;
R₂ is selected from the group consisting of hydrogen, methyl, ethyl and a —CH₂—O—C(O)—R₁ₐ;
R₃ is selected from the group consisting of hydrogen, aryl, arylene-alkyl, alkylene-aryl and linear C1-C6 alkyl group;
R₁ₐ is selected from the group consisting of linear or branched C1-C19 alkyl, linear or branched C1-C19 alkenyl, aryl, arylene-alkyl and alkylene-aryl group, wherein said alkyl or alkenyl group optionally contains up to 5 heteroatoms, independently selected from O and N;
each Rₓ is independently selected from the group consisting of methyl, ethyl, propyl and —CH₂—CH₂—OH;
each R_{y} group is independently selected from the group consisting of hydrogen, linear or branched C1-C18 alkyl, aryl, arylene-alkyl or alkylene-aryl group, wherein said alkyl group optionally contains up to 5 heteroatoms, independently selected from O and N;
or rotamers, tautomers stereoisomers or regioisomers thereof.

21. The lipid nanoparticle according to claim 10, wherein:
the amount of the modified apolipoprotein (in case of two or more modified apolipoproteins, cumulatively) ranges from 0.08 to 2.0 mol %, such as from 0.10 to 2.0 mol %; and/or
the amount of phospholipid ranges from 5 to 90 mol %, such as from 15 to 90 mol %; and/or
the amount of sterol ranges from 2.5 to 65 mol %, such as from 2.5 to 50 mol %; and/or
the amount of cationic or ionizable cationic lipid ranges from 5.0 to 80 mol %, such as from 8.0 to 80 mol %,
wherein the molar percentage is based solely on the combined amounts of the modified apolipoprotein, phospholipids, sterols and cationic or ionizable cationic lipids in the lipid nanoparticle.

22. The lipid nanoparticle according to claim 10, wherein:
the amount of the modified apolipoprotein (in case of two or more modified apolipoproteins, cumulatively) ranges from 0.1 to 90 weight %;
the amount of nucleic acid ranges from 0.01 to 90 weight %;
the amount of phospholipid ranges from 0.1 to 95 weight %;
the amount of sterol ranges from 0.1 to 95 weight %; and/or
the amount of cationic and/or ionizable cationic lipid ranges from 0.1 to 95 weight %,
wherein these weight percentages are based on the combined amounts of the modified apolipoprotein, the nucleic acid, the phospholipid, the sterol and the cationic and/or ionizable cationic lipid.

23. The lipid nanoparticle according to claim 10, wherein the ratio of the modified apolipoprotein (in case of two or more modified apolipoproteins, cumulatively) to phospholipid based on percentage molar weight is between 1:25 and 1:400, more preferably between 1:50 and 1:200, even more preferably between 1:75 and 1:150.

24. The lipid nanoparticle according to claim 10, wherein the ratio of the modified apolipoprotein (in case of two or more modified apolipoproteins, cumulatively) to phospholipid based on weight is from 2:1 to 1:10, more preferably from 1:1 to 1:5, even more preferably from 1:1.5 to 1:4.

25. Method of manufacturing a lipid nanoparticle as defined in claim 7, the method comprising the steps of:
a1) expressing and isolating a modified apolipoprotein as defined in any one of claims 1-5 to obtain an isolated modified apolipoprotein; and/or
a2) chemically conjugating a targeting body to an apolipoprotein or apolipoprotein mimetic to obtain a modified apolipoprotein and isolating the modified apolipoprotein;
b) combining the isolated modified apolipoprotein obtained in step a1 and/or step a2 with phospholipids, sterols and optionally lipids to obtain a lipid nanoparticle.

26. The method of claim 25 wherein step b) comprises:
b1) mixing, preferably rapid mixing, of lipid components in organic solvent with a nucleic acid in an aqueous buffer to produce nanoparticles, wherein the lipid components comprise a phospholipid, a sterol, a core component, and optionally a lipid; and wherein the aqueous buffer has a pH of 5.5 or lower, preferably 5.0 or lower; and
b2) mixing, preferably rapid mixing, of lipid nanoparticles with the modified apolipoprotein to produce the nanoparticle at a pH between 5.5 and 9.0, preferably at a pH between 6.0 and 8.0, more preferably at a pH between 6.5 and 8.0.

27. Method for producing a lipid nanoparticle, comprising the step of:
a) rapid mixing of lipid components in organic solvent with a nucleic acid in an aqueous buffer to produce lipid nanoparticles, wherein the lipid components comprise a phospholipid, a sterol, a cationic lipid or ionizable cationic lipid, wherein the aqueous buffer has a pH of 5.0 or lower; and
b) rapid mixing of the lipid nanoparticles with one or more modified apolipoprotein as defined in claim 1;
to produce the lipid nanoparticle at a pH between 5.5 and 8.0, preferably pH between 6.0 and 8.0.

28. (canceled)

29. A method of treating or preventing an immune related disorder in a subject in need thereof, comprising administering to the subject the lipid nanoparticle according to claim 7, wherein, optionally, the immune related disorder is transplantation rejection, graft-versus-host disease (GVH), atherosclerosis, infection, inflammation, auto-immunity, allergy, cancer, a genetic disorder, a metabolic disorder, a neurological disorder or tissue trauma.

30. A method of delivering a compound to a target, comprising delivering the compound to the target in a lipid nanoparticle according to claim 7, preferably wherein the target is a cell, tissue, and/or organ, even more preferably wherein the target is a lymphoid cell, a myeloid cell, a tumor cell, an endothelial cell, a hematopoietic stem and progenitor cell (HSPC), a hematopoietic stem cell (HSC), a multipotent progenitor (MPP), a common myeloid progenitor cell (CMP), or wherein the targeting body binds a bacterial, viral, fungal or parasitic protein or antigen,
preferably wherein the lymphoid or myeloid cell is selected from: a monocyte, a macrophage, an M1-like macrophage, an M2-like macrophage, an eosinophil, a basophil, a mast cell, an NK cell, a B cell, a plasma cell, a regulatory T cell, a hematopoietic stem cell, a T helper cell such as Th1, Th2, Th17 or Th22, a dendritic cell, such as a plasmacytoid dendritic cell, a conventional DC 1 or a conventional DC 2, or a tumor-associated macrophage.

31. An in vitro or ex vivo method for introducing a nucleic acid in a cell, the method comprising contacting the lipid nanoparticle according to claim 9, with a cell.

32. An in vivo method for introducing a nucleic acid in a cell, the method comprising contacting the lipid nanoparticle according to claim 9, with a cell.

33. (canceled)

34. A method for the in vivo delivery of a nucleic acid, the method comprising administering the lipid nanoparticle according claim 9, to a subject.

35. A method for treating a disease or disorder in a subject in need thereof, the method comprising administering a therapeutically effective amount of the lipid nanoparticle according to claim 9, to the subject.

36. The method according to claim 35, wherein the disease is an immune related disorder such as transplantation rejection, graft-versus-host disease (GVH), atherosclerosis, infection, inflammation, auto-immunity, allergy, cancer, a genetic disorder, a metabolic disorder, a neurological disorder or tissue trauma.
